SEQUENCE LISTING (4 sequences)

SEQ ID NO: 1 - DNA, 21 bp, Artificial
  PCR primer
cttccagacagcgagggagaa

SEQ ID NO: 2 - DNA, 21 bp, Artificial
  PCR primer
tctccatggggcaggatgtct

SEQ ID NO: 3 - DNA, 21 bp, Artificial
  PCR primer
atgagcctttccttctgtggt

SEQ ID NO: 4 - DNA, 22 bp, Artificial
  PCR primer
cgcccgttgcgagtctgaaaca

(19) United States
(12) Patent Application Publication
(10) Pub. No.: US 2007/0026079 A1
(43) Pub. Date: Feb. 1, 2007

(54) Intranasal administration of modulators of hypothalamic ATP-sensitive potassium channels

(76) Inventors: Louis Herlands, Cambridge, MA (US); Luciano Rossetti, Bedford Hills, NY (US); Alessandro Pocai, New York, NY (US)

Correspondence Address: MYERS BIGEL SIBLEY & SAJOVEC, PO BOX 37428, RALEIGH, NC, 27627, US

(21) Appl. No.: 11/353,594

(22) Filed: Feb. 13, 2006

Related U.S. Application Data
(60) Provisional application No. 60/652,840, filed on Feb. 14, 2005.
(60) Provisional application No. 60/652,592, filed on Feb. 14, 2005.

(51) Int. Cl. A61K 31/549; A61K 31/513; A61K 9/14

(52) U.S. Cl. 424/489; 514/223.2; 514/269

(57) ABSTRACT

Provided are methods of increasing K_{ATP} channel activity in the hypothalamus of a mammal, methods of reducing glucose production in a mammal, methods of reducing peripheral glucose levels in a mammal, methods of reducing triglyceride levels in a mammal, methods of reducing very low density lipoprotein (VLDL) levels in a mammal, methods of methods of reducing gluconeogenesis in the liver of a mammal, methods of treating metabolic disorders such as diabetes, hyperglycemia, insulin resistance, glucose intolerance, metabolic syndrome and/or obesity, and methods of increasing glucose production and peripheral glucose levels in a mammal. Also provided are methods of treating heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, polycystic ovary syndrome, gonadotropin deficiency and/or amenorrhea.

### RELATED APPLICATION INFORMATION

This application claims the benefit of U.S. Provisional Application Ser. Nos. 60/652,840 and 60/652,592, filed Feb. 14, 2005, the disclosures of which are incorporated herein by reference in their entireties.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of DK 48321, DK 45024, DK 066058, and DK 47208 awarded by the National Institutes of Health.

### BACKGROUND OF THE INVENTION

(1) Field of the Invention

The present invention generally relates to regulation of glucose production in mammals. More specifically, the invention relates to the modulation of hepatic glucose production through the activation or inhibition of ATP-sensitive potassium (K_{ATP}) channels.

(2) Description of the Related Art

### REFERENCES CITED

L. Aguilar-Bryan and J. Bryan, Endocr. Rev. 20,101-135 (1999).
L. Aguilar-Bryan et al., Science 268, 423-426 (1995).
N. Barzilai et al., J. Clin. Invest 100, 3105-3110 (1997).
R. D. Carr et al., Diabetes 52, 2513-2518 (2003).
J. C. Bruning et al., Science 289, 2122-2125 (2000).
W. A. Chutkow et al., Proc. Natl. Acad. Sci. U.S.A 98, 11760-11764 (2001).
J. S. Flier, Cell 116, 337-350 (2004).
J. M. Friedman, Science 299, 856-858 (2003).
H. J. Grill et al., Endocrinology 143, 239-246 (2002).
N. Inagaki et al., Neuron 16, 1011-1017 (1996).
C. Karschin, C. Ecke, F. M. Ashcroft, A. Karschin, FEBS Lett. 401, 59-64 (1997).
P. G. Kopelman, Nature 404, 635-643 (2000).
S. E. Ia Fleur, H. Ji, S. L. Manalo, M. I. Friedman, M. F. Dallman, Diabetes 52, 2321-2330 (2003).
L. Liu et al., J. Biol. Chem. 273, 31160-31167 (1998).
T. M. Loftus et al., Science 288, 2379-2381 (2000).
I. Magnusson, D. L. Rothman, L. D. Katz, R. G. Shulman, G. I. Shulman, J. Clin. Invest 90, 1323-1327 (1992).
M. Matsuhisa et al., Metabolism 49, 11-16 (2000).
Y. Minokoshi et al., Nature 428, 569-574 (2004).
M. C. Moore et al., Am. J. Physiol Endocrinol. Metab 282, E286-E296 (2002).
G. J. Morton et al., Endocrinology 144, 2016-2024 (2003).
K. D. Niswender et al., Diabetes 52, 227-231 (2003).
R. Norgren and G. P. Smith, J. Comp Neurol. 273, 207-223 (1988).
S. Obici, B. B. Zhang, G. Karkanias, L. Rossetti, Nat. Med 8, 1376-1382 (2002a).
S. Obici, Z. Feng, G. Karkanias, D. G. Baskin, L. Rossetti, Nat. Neurosci. 5, 566-572 (2002b).
S. Obici et al., Diabetes 51, 271-275 (2002c).
S. Obici, Z. Feng, A. Arduini, R. Conti, L. Rossetti, Nat. Med 9, 756-761 (2003).
J. Pijewski, M. Kotlihski, http://www.nencki.gov.pl/iiclab/db.html (2000).
A. Pocai, S. Obici, G. J. Schwartz, L. Rossetti, Cell Metab, in press.
D. L. Rothman, I. Magnusson, L. D. Katz, R. G. Shulman, G. I. Shulman, Science 254, 573-576 (1991).
M. W. Schwartz, S. C. Woods, D. Porte, Jr., R. J. Seeley, D. G. Baskin, Nature 404, 661-671 (2000).
V. Seghers, M. Nakazaki, F. DeMayo, L. Aguilar-Bryan, J. Bryan, J. Biol. Chem. 275, 9270-9277 (2000).
V. Seghers, M. Nakazaki, F. DeMayo, L. Aguilar-Bryan, J. Bryan, J. Biol. Chem. 275, 9270-9277 (2000).
S. Seino and T. Miki, Prog. Biophys. Mol. Biol. 81, 133-176 (2003).
S. Seino, T. Iwanaga, K. Nagashima, T. Miki, Diabetes 49, 311-318 (2000).
D. Spanswick, M. A. Smith, V. E. Groppi, S. D. Logan, M. L. Ashford, Nature 390, 521-525 (1997).
D. Spanswick, M. A. Smith, S. Mirshamsi, V. H. Routh, M. L. Ashford, Nat. Neurosci. 3, 757-758 (2000).
S. C. Woods, E. C. Lotter, L. D. McKay, D. Porte, Jr., Nature 282, 503-505 (1979).
S. C. Woods, R. J. Seeley, D. Porte, Jr., M. W. Schwartz, Science 280, 1378-1383 (1998).
U.S. Pat. No. 6,197,765.
U.S. Pat. No. 6,147,098.
U.S. Pat. No. 5,889,002.
U.S. Pat. No. 5,284,845.
U.S. Pat. No. 4,921,706.
U.S. Pat. No. 4,895,452.
U.S. Provisional Application Ser. No. 60/677,708.
U.S. Provisional Application Ser. No. 60/677,707.
U.S. Provisional Application Ser. No. 60/694,111.

Obesity is the driving force behind the dramatic worldwide increase in the prevalence of type 2 diabetes mellitus (DM2) (Friedman, 2003; Kopelman, 2000; Flier, 2004). Fasting hyperglycemia is the hallmark of DM and it is largely due to a marked increase in the rate of hepatic gluconeogenesis (Rothman et al., 1991; Magnusson et al., 1992). Recent reports have identified the medial hypothalamus as a major integrator of nutritional and hormonal signals (Friedman, 2003; Flier, 2004; Schwartz et al., 2000; Woods et al., 1998), which play pivotal roles not only in the regulation of energy balance but also in the modulation of liver glucose output (Obici et al., 2002a; 2003). In this regard, bi-directional changes in hypothalamic insulin signaling results in parallel changes in both energy balance (Obici et al., 2002b; Bruning et al., 2000; Woods et al., 1979; Niswender et al., 2003) and glucose metabolism (Obici et al., 2002a). ATP-sensitive potassium (K_{ATP}) channels are expressed in the hypothalamus (Karschin et al., 1997) and can be activated by insulin (and leptin) in selective hypothalamic neurons (Spanswick et al., 1997; 2000). However, while it has been postulated that this central action of insulin could mediate some of its rapid effects (Spanswick et al., 2000), the functional role of insulin's activation of hypothalamic K_{ATP} channels remains obscure. The present invention is based on the discovery of that role.

### SUMMARY OF THE INVENTION

The inventors have discovered that activation of hypothalamic K_{ATP} channels causes a reduction in peripheral blood glucose levels and glucose production. The present invention provides methods of intranasally administering a K_{ATP} channel activator or inhibitor to the central nervous system (CNS) (for example, the brain, the hypothalamus [e.g., mediobasal hypothalamus including the arcuate nucleus]), thereby avoiding the need for invasive modes of administration directly to the CNS. Also provided are pharmaceutical compositions formulated for intranasal delivery comprising one or more K_{ATP} channel activators or inhibitors.

Accordingly, as a first aspect, the invention provides a method of reducing peripheral blood glucose levels in a mammal, the method comprising intranasally administering a K_{ATP} channel activator to the hypothalamus of the mammal in an amount effective to reduce peripheral blood glucose levels in the mammal.

The invention also provides a method of reducing glucose production in a mammal, the method comprising intranasally administering a K_{ATP} channel activator to the hypothalamus of the mammal in an amount effective to reduce glucose production in the mammal.

As yet a further aspect, the invention provides a method of reducing gluconeogenesis in the liver of a mammal, the method comprising intranasally administering a K_{ATP} channel activator to the hypothalamus of the mammal in an amount effective to reduce hepatic gluconeogenesis in the mammal.

The invention also provides a method of reducing serum triglyceride levels in a mammal, the method comprising intranasally administering a K_{ATP} channel activator to the hypothalamus of the mammal in an amount effective to reduce serum triglyceride levels in the mammal.

Still another aspect of the invention is a method of reducing serum very low density lipoprotein (VLDL) levels in a mammal, the method comprising intranasally administering a K_{ATP} channel activator to the hypothalamus of the mammal in an amount effective to reduce serum VLDL levels in the mammal.

Further provided is a method of treating a disorder in a mammal selected from the group consisting of obesity, type 2 diabetes, type 1 diabetes, hyperglycemia, insulin resistance, glucose intolerance, leptin resistance, metabolic syndrome, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia (e.g., total serum cholesterol greater than 240 mg/dl and/or serum LDH greater than 130 mg/dl and, optionally, serum HDL less than 30 mg/dl), hypertension (e.g., systolic blood pressure greater than 140 and/or diastolic blood pressure less than 90), and any combination of the foregoing, the method comprising intranasally administering a K_{ATP} channel activator to the hypothalamus of the mammal in an amount effective to treat the disorder.

As still a further aspect, the invention provides a method of increasing K_{ATP} channel activity in the hypothalamus of a mammal, the method comprising intranasally administering a K_{ATP} channel activator to the mammal in an amount effective to increase K_{ATP} channel activity in the hypothalamus.

The invention also encompasses methods of reducing hypothalamic K_{ATP} channel activity in a mammal, increasing peripheral blood glucose levels in a mammal, increasing glucose production in a mammal, increasing hepatic gluconeogenesis in a mammal, and/or treating hypoglycemia in a mammal, the methods comprising intranasally administering a K_{ATP} channel inhibitor to the hypothalamus of the mammal in an amount effective to reduce hypothalamic K_{ATP} channel activity, increase peripheral blood glucose levels, increase glucose production, increase hepatic gluconeogenesis, and/or treat hypoglycemia in the mammal.

In other embodiments, the invention provides a pharmaceutical composition formulated for intranasal administration comprising one or more K_{ATP} channel activators or inhibitors in a pharmaceutically acceptable carrier.

Also provided is the use of a compound or pharmaceutical composition of the invention for activating or inhibiting hypothalamic K_{ATP} channels, reducing or increasing glucose production, reducing or increasing glucose levels, reducing or increasing gluconeogenesis, and/or for treating diabetes, metabolic syndrome, hyperglycemia, insulin resistance, glucose intolerance, leptin resistance, gonadotropin deficiency, amenorrhea, polycystic ovary syndrome, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension and/or obesity or hypoglycemia.

These and other aspects of the invention are set forth in more detail below.

### BRIEF DESCRIPTION OF THE DRAWINGS

FIG. 1 shows graphs demonstrating that activation of hypothalamic K_{ATP} channels lowers blood glucose via inhibition of glucose production (GP). Panel A shows that ICV infusion of Diazoxide (Diaz) decreases plasma glucose levels in fasted (5 h) rats. All rats received ICV infusion of either Diaz or vehicle (Veh) during the 6 h studies. At 120 min, an infusion of [³H]glucose was initiated. The pancreatic-insulin clamp study was initiated at 240 min. This involved the infusion of somatostatin (3 μg/kg per min), insulin (1 mU/kg per min) and glucose (as needed to prevent hypoglycemia). Panel B shows that, when ICV Diaz was infused, the systemic infusion of glucose was required in order to prevent hypoglycemia. ICV Diaz markedly inhibited GP during the pancreatic clamp. Panel C shows that ICV Diaz markedly suppresses the flux through G6 Pase and the hepatic expression of G6 Pase.

Panel D shows that central opening of K_{ATP} channels inhibits gluconeogenesis and the hepatic expression of PEPCK. Intrahvpothalamic (IH) infusions of diazoxide and insulin. To localize the effect of central Diaz and insulin on GP, the pancreatic-insulin clamp studies were repeated in rats with bilateral cannulae implanted within the parenchyma of the mediobasal hypothalamus. Panels E and G show that IH infusion of either Diaz or Insulin (Ins) decreases plasma glucose levels. Panel F and H show that IH infusion of either Diaz or Ins during a pancreatic clamp led to significant increases in the rate of glucose infusion required to prevent hypoglycemia and to marked suppression of GP compared with vehicle-infused controls. *P<0.05 vs vehicle-infused controls.

FIG. 2 shows graphs and a photograph of an electrophoretic gel demonstrating that central insulin lowers blood glucose and suppresses GP via activation of SUR1-containing K_{ATP} channels. Panel A shows a schematic representation of the clamp procedure. The ICV infusion of a K_{ATP} channel blocker negates the blood glucose lowering effect of central insulin. Panel B shows that, during a pancreatic-clamp, ICV insulin (Ins, □) but not ICV Vehicle (Veh, □) inhibited GP. This effect of insulin was abolished by the co-infusion of a K_{ATP} channel blocker (□) while infusion of either K_{ATP} channel blocker or Veh (□) did not per se affect GP. Panel C shows that the ICV administration of a K_{ATP} channel blocker prevents the inhibitory effects of central insulin on the flux through G6 Pase and on the hepatic expression of G6 Pase. Panel D shows that central administration of a K_{ATP} channel blocker negates the inhibitory effects of insulin on gluconeogenesis and on the hepatic expression of PEPCK. Panel E shows the expression of SUR1 and SUR2 transcripts in hypothalamic nuclei. Expression of SUR1 or SUR2 was examined using PCR in arcuate (ARC) and paraventricular nuclei (PVN) of the hypothalamus and in the lateral hypothalamic area (LHA). PCR results from the following cells and tissues were included as controls: rat pancreatic islets (ISL), mouse beta-TC-3 cells (β-TC3) and rat heart (HRT). Panel G shows an experimental scheme used to establish that SUR1 KO mice display selective impairment in hepatic insulin action. Euglycemic-hyperinsulinemic clamp studies were performed in conscious mice. The infusion studies lasted a total of 90 minutes. Briefly, at 0 minutes, insulin was infused at the rate of 3.6 mU/min·kg body weight and a solution of glucose (10% wt/vol) was infused at a variable rate as required to maintain euglycemia (8 mM). Mice also received a constant infusion of HPLC-purified [³H-3]-glucose. As shown in Panel F, in the presence of physiological hyperinsulinemia, Sur1 KO mice displayed increased rates of GP and Gluconeogenesis (H) while the rates of glucose uptake (F) and Glycogenolysis were not significantly altered compared with WT mice (H). *P<0.05 vs vehicle controls.

FIG. 3 shows illustrations and graphs demonstrating that the effect of central insulin on hepatic glucose homeostasis requires descending fibers within the hepatic branch of the vagus nerve. Panel A is an illustration of hepatic vagotomy. A laparotomy incision is made on the ventral midline and the abdominal muscle wall is opened with a second incision, revealing the gastrointestinal tract in the peritoneum. The gastrohepatic ligament is severed using fine forceps, and the stomach is gently retracted onto sterile saline soaked cotton gauze, revealing the descending ventral esophagus and the ventral subdiaphragmatic vagal trunk. The hepatic branch of this vagal trunk is then transected by microcautery. Panel B shows that, during a pancreatic-clamp, ICV insulin (□, vehicle; □, insulin) increases the rate of glucose infusion and inhibits GP in SHAM but not in HV rats. HV did not per se affect either glucose infusion or GP. Panel C shows that HV also negates the inhibitory effects of central insulin on the flux through G6 Pase and on the hepatic expression of G6 Pase (o, vehicle; □, insulin). Panel D shows that HV also negates the inhibitory effects of central insulin on gluconeogenesis and on the hepatic expression of PEPCK. Panel E is an illustration of selective vagal deafferentation. The afferent vagus branch derived from the right abdomen is resected at the site of entry in the brainstem (see methods). Panel F shows that vagal deafferentation does not alter the ability of central insulin to increase the rate of glucose infusion and to suppress GP. *P<0.05 vs vehicle controls.

FIG. 4 shows illustrations and graphs demonstrating that the hepatic branch of the vagus nerve is required for the effect of physiological hyperinsulinemia on GP and gluconeogenesis. Panel A is a schematic representation of the pancreatic-hyperinsulinemic clamp procedure. At t=120 an infusion of labeled glucose was initiated and maintained for the remaining 4 hours of the study and a pancreatic hyperinsulinemic clamp was initiated at t=240 min and lasted for 2 hours. The latter procedure involved the infusion of somatostatin (SRIF; 3 μg/kg/min), insulin (3 mU/kg·min), and glucose as needed to prevent hypoglycemia.

Panel B shows that hepatic vagotomy (HV) induces severe hepatic insulin resistance. During the pancreatic-insulin clamp study, the rate of glucose infusion and the suppressive effect of insulin on GP were markedly diminished in HV rats compared with SHAM rats (□, vehicle; □, insulin). Panel C shows that HV also impairs the inhibitory effects of systemic insulin on the flux through G6 Pase and on the hepatic expression of G6 Pase. Panel D shows that HV abolishes the inhibitory effects of insulin on gluconeogenesis and on the expression of PEPCK. Panel E is a schematic summary of the neuronal and metabolic mechanisms by which the central activation of K_{ATP} channels decreases GP and gluconeogenesis. The figure is a longitudinal view of a rat brain, with olfactory bulb at the anterior end on the left and the caudal hindbrain on the right. A coronal section of the brain at the level of the arcuate nuclei shows the positioning of one of the infusion cannulae used for the IH studies. Insulin and diazoxide were directly infused within the parenchyma of the mediobasal hypothalamus (ARC) leading to activation of K_{ATP} channels and to dramatic changes in hepatic gene expression and metabolism. The central effects of insulin on liver gene expression and metabolism were completely abolished by the central administration of a K_{ATP} channel blocker (K_{ATP} blocker) and by the surgical resection of the hepatic branch of the vagus nerve. Thus, these findings suggest that the activation of neuronal projections from the hypothalamus to brainstem nuclei (DMX and NTS) generates an efferent vagal impulse to the liver, which is specifically required for the inhibition of glucose production and gluconeogenesis and of the hepatic expression of G6 Pase and PEPCK.*P<0.05 vs. vehicle controls.

FIG. 5 shows results using a certain experimental protocol. Panel A left shows a schematic of the experimental protocol. ICV cannulae were surgically implanted on day 1 (3 weeks before the in vivo study). Full recovery of body weight and food intake was achieved by day 7. Clamp studies were done on day 21. Panel A right shows a schematic outline of the major pathways and enzymatic steps contributing to glucose production. Hepatic glucose-6-phosphate pool (glucose-6-P) is the result of three major fluxes: (1) plasma-derived glucose, 2) gluconeogenesis, and (3) glycogenolysis. The final common pathway for hepatic glucose output is the net dephosphorylation of glucose-6-P, which results from the balance of glucokinase (GK) and Glucose-6-phosphatase (G6 Pase) activities. Similarly, the net contribution of hepatic glycogen to the G6P pool represents the balance of the fluxes through glycogen synthase and glycogen phosphorylase. The relative contribution of plasma glucose and gluconeogenesis to the hepatic glucose G6P pool can be directly measured by tracer methodology. Briefly, after [³H]glucose infusion, the ratio of specific activities of tritiated hepatic UDP-glucose plasma glucose represents the percent of hepatic G6P pool which is derived from plasma glucose. Similarly, after [¹⁴C]lactate infusion, the proportion of the G6P pool which is formed through gluconeogenesis (PEP) can be calculated as the ratio of [¹⁴C]-labeled UDP-glucose and PEP. Panel B shows the rate of glucose uptake before (□) and during pancreatic-insulin clamp studies in rats treated with ICV administration of diazoxide (Diaz, □) compared with vehicle controls (Veh, □). The rate of glucose disposal was not significantly affected by ICV treatment. Panel C shows the rates of glucose production (GP) during pancreatic-insulin clamp studies in rats treated with ICV administration of Diaz (□) compared with appropriate control (□). Panel D shows that ICV infusion of Diazoxide significantly reduced glucose cycling while the rate of glycogenolysis was not decreased.

FIG. 6 shows the results using another protocol. Panel A shows a schematic of the protocol. ICV cannulae were surgically implanted on day 1 (3 weeks before the in vivo study). Full recovery of body weight and food intake was achieved by day 7. Clamp studies were done on day 21. Panel B shows the rate of glucose uptake before (□) and during pancreatic-insulin clamp studies in rats treated with ICV administration of insulin or insulin plus K_{ATP} blocker (□) compared with respective controls (□). The rate of glucose disposal was not significantly affected by ICV treatment. Panel C shows the rates of glucose production (GP) during pancreatic-insulin clamp studies in rats treated with ICV administration of insulin or insulin plus K_{ATP} blocker (□) compared with their appropriate controls (□). Panel D shows that ICV K_{ATP}-blocker blocks the suppressive effect of insulin on glucose cycling. The rate of glycogenolysis was not decreased by ICV infusion of insulin.

FIG. 7 shows the results using still another protocol. Panel A shows a schematic of the protocol. ICV cannulae were surgically implanted on day 1 (3 weeks before the in vivo study). Full recovery of body weight and food intake was achieved by day 7. One week before the pancreatic-insulin clamp protocols (day 21), rats underwent selective hepatic vagotomy and received additional catheters in the right internal jugular and left carotid artery. Clamp studies were done on day 21. Panel B shows the rate of glucose uptake before (□) and during pancreatic-insulin clamp studies in rats treated with ICV administration of insulin (□) compared with vehicle controls (□) in rats subjected to hepatic vagotomy (HV) or sham operation (Sham). The rate of glucose disposal was not significantly affected by ICV treatment. Panel C shows the rates of glucose production (GP) during pancreatic-insulin clamp studies in rats treated with ICV administration of insulin (□) compared with appropriate control (□). Panel D shows that HV blocks the suppressive effect of insulin on glucose cycling. The rate of glycogenolysis was not decreased by ICV infusion of insulin.

FIG. 8 shows the results using an additional protocol. Panel A shows a schematic of the protocol. One week before the pancreatic-insulin clamp protocols (day 21), rats underwent selective hepatic vagotomy (HV) or sham operation (Sham) and received catheters in the right internal jugular and left carotid artery. Clamp studies were done on day 21. Panel B shows the rate of glucose uptake before (□) and during hyperinsulinemic-euglycemic clamp studies (increase ˜3-fold over basal levels) in rats HV or Sham. The rate of glucose disposal was not significantly affected by HV. Panel C shows that HV (□) reduces the suppressive effect of systemic insulin on glucose production (GP) compared with appropriate control (□). Panel D shows that HV reduces the suppressive effect of systemic insulin on glucose cycling. The rate of glycogenolysis was not affected by HV.

FIG. 9 shows validation of the placement of an intrahypothalamic cannulae. To verify the correct anatomical placement of the bilateral cannulae within the parenchyma of the medial hypothalamus radioactive tracers (³H-glucose and ³H-Glybenclamide) were infused during the clamp followed by sampling of hypothalamic nuclei obtained by micropunches. Radioactive tracer was found only in the arcuate (ARC), while no counts were found in paraventricular nucleus (PVN) and lateral hypothalamic area (LHA).

### DETAILED DESCRIPTION OF THE INVENTION

The present invention will now be described with reference to the accompanying drawings, in which preferred embodiments of the invention are shown. This invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used in the description of the invention and the appended claims, the singular forms “a,” “an” and “the” are intended to include the plural forms as well, unless the context clearly indicates otherwise.

The present invention is based, in part, on the inventors' discovery that activating hypothalamic ATP-sensitive potassium (K_{ATP}) channels decreases glucose production and blood glucose levels, and inhibiting hypothalamic K_{ATP} channels has the opposite effect. Without being bound by any particular mechanism, it is believed that the reduction in peripheral blood glucose levels and glucose production is caused by a decrease in hepatic gluconeogenesis, which appears to involve signaling through the efferent vagus nerve. The effect is believed to be concentrated in the mediobasal hypothalamus, including the arcuate nucleus. Thus, glucose production can be controlled by activating or inhibiting K_{ATP} channels in the CNS.

In addition, in embodiments of the invention, K_{ATP} channel activation results in a reduction in serum lipids, including serum cholesterol, serum triglycerides and/or serum VLDL. Co-pending U.S. Provisional Application Ser. Nos. 60/677,708, 60/677,707 and 60/694,111 demonstrate that manipulation of hypothalamic amino acid or lactate levels produces a decrease in serum triglycerides and VLDL.

The invention is further based, in part, on the recognition that K_{ATP} channel activators can be administered intranasally to the CNS (for example, the brain, the hypothalamus [e.g., the mediobasal hypothalamus, including the ARC]) of a mammal, to increase K_{ATP} channel activity in the CNS, to lower peripheral blood glucose levels, to reduce glucose production, to reduce gluconeogenesis, to treat metabolic disorders such as diabetes (type 1 and/or type 2), hyperglycemia, insulin resistance, glucose intolerance, leptin resistance, metabolic syndrome, obesity, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension and/or to treat gonadotropin deficiency, amenorrhea, and/or polycystic ovary syndrome. Further, the invention is based in part on the appreciation by the inventors that K_{ATP} channel inhibitors can be administered intranasally to the CNS of a mammal to decrease K_{ATP} channel activity in the CNS, to increase glucose production, to increase peripheral blood glucose levels and/or to treat hypoglycemia and/or as a treatment for a mammal undergoing a therapy that causes insufficient food intake and/or loss of appetite and/or glucose production (such as chemotherapy) or as a treatment for a mammal that otherwise has insufficient glucose production (e.g., because of a viral infection).

The compositions and methods of the present invention provide for the delivery of compounds to the CNS (for example, the brain or the hypothalamus (e.g., the ARC)) by the nasal route, while minimizing systemic exposure. In this regard and without being bound to any particular theory, it is believed that targeting the CNS by nasal administration is based on capture and internalization of substances by the olfactory receptor neurons, which substances are then transported inside the neuron to the olfactory bulb of the brain. Olfactory receptor neurons from the lateral olfactory tract within the olfactory bulb project to various regions such as the hippocampus, amygdala, thalamus, hypothalamus and other regions of the brain that are not directly involved in olfaction. These substances may also pass through junctions in the olfactory epithelium at the olfactory bulb and enter the subarachnoid space, which surrounds the brain, and the cerebral spinal fluid (CSF), which bathes the brain. Either pathway allows for targeted delivery without interference by the blood brain barrier, as neurons and the CSF, not the circulatory system, are involved in these transport mechanisms. Accordingly, intranasal delivery pathways permit compartmentalized delivery of compositions with substantially reduced systemic exposure and the resulting side effects.

As further advantages, nasal delivery offers a noninvasive means of administration that is safe and convenient for self-medication. Intranasal administration can also provide for rapid onset of action due to rapid absorption by the nasal mucosa. This characteristic of nasal delivery result from several factors, including: (1) the nasal cavity has a relatively large surface area of about 150 cm² in man, (2) the submucosa of the lateral wall of the nasal cavity is richly supplied with vasculature, and (3) the nasal epithelium provides for a relatively high drug permeation capability due to thin single cellular layer absorption.

K_{ATP} channels are found in many tissues, including skeletal and smooth muscle, heart, pancreatic β-cells, pituitary, and brain. These channels are thought to regulate various cellular functions such as hormone secretion, excitability of neurons and muscles, and cytoprotection during ischemia by coupling cell metabolism to membrane potential. The K_{ATP} channels in pancreatic β-cells are critical metabolic sensors that determine glucose-responsive membrane excitability in the regulation of insulin secretion. In the brain, K_{ATP} channels have been found in many regions, including substantia nigra, neocortex, hippocampus and hypothalamus.

The K_{ATP} channel is an octameric protein consisting of two subunits: the pore-forming inward rectifier K⁺ channel member Kir6.1 or Kir6.2, and the sulfonylurea receptor SUR1 or SUR2 (SUR2A, SUR2B or possibly other SUR2 splice variants). Whereas pancreatic β-cell K_{ATP} channels comprise Kir6.2 and SUR1, cardiac K_{ATP} channels comprise Kir6.2 and SUR2A. For different neuronal populations, all possible co-expression patterns of Kir6.1 or Kir6.2 and SUR1 or SUR2A have been reported.

As used herein, the term “K_{ATP} channel” can refer to any K_{ATP} channel now known or later discovered, and encompasses any combination of Kir6.1 or Kir6.2 with SUR1 or SUR2 (including SUR2A or SUR2B), as well as variants of any of the foregoing. In particular embodiments, the K_{ATP} channel is a Kir6.1/SUR1 and/or Kir6.2/SUR1 type channel. In other embodiments, the K_{ATP} channel is a Kir6.2/SUR2 (including Kir6.2/SUR2A and/or Kir6.2/SUR2B) type channel.

Applications of the Present Invention.

The present invention finds use in research as well as veterinary and medical applications. Suitable subjects are generally mammalian subjects. The term “mammal” as used herein includes, but is not limited to, humans, non-human primates, cattle, sheep, goats, pigs, horses, cats, dog, rabbits, rodents (e.g., rats or mice), etc.

In particular embodiments, the subject is a human subject that has been diagnosed with or is considered at risk for a metabolic disorder such as diabetes (e.g., type 1 or type 2), metabolic syndrome, hyperglycemia, insulin resistance, glucose intolerance, leptin resistance, heart failure, ischemia, coronary heart disease, familial lipoprotein lipase deficiency, hypopituitarism, hyperlipidemia, hypertriglyceridemia, hyperVLDLemia (i.e., elevated VLDL levels), atherosclerosis, hypercholesterolemia, hypertension and/or obesity. The subject can further be a human subject that desires to lose weight for cosmetic and/or medical reasons. Further, the subject can be a human subject that has been diagnosed with or is considered at risk for gonadotropin deficiency, amenorrhea, and/or polycystic ovary syndrome. Human subjects include neonates, infants, juveniles, and adults.

In other embodiments, the subject used in the methods of the invention is an animal model of diabetes, hyperglycemia, metabolic syndrome, obesity, glucose intolerance, insulin resistance, leptin resistance, gonadotropin deficiency, amenorrhea, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension and/or polycystic ovary syndrome.

In particular embodiments of the invention, the subject is a subject “in need of” the methods of the present invention, e.g., in need of the therapeutic effects of the inventive methods. For example, the subject can be a subject that has been diagnosed with or is considered at risk for diabetes (type 1 or type 2), metabolic syndrome, hyperglycemia, insulin resistance, glucose intolerance, obesity, leptin resistance, gonadotropin deficiency, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, amenorrhea, and/or polycystic ovary syndrome, and the methods of the invention are practiced on the subject as a method of prophylactic or therapeutic treatment.

As used herein, the terms “delivery to,” “administering to,” “administration to” or “activation [inhibition] of K_{ATP} channels in” the hypothalamus (and similar terms) can refer to the hypothalamus when assessed as a whole, or can refer to particular regions of the hypothalamus (e.g., the mediobasal hypothalamus or the ARC). Administration to the hypothalamus can be by any route including by peripheral or central administration routes. In particular embodiments, administration to the hypothalamus is by an intranasal route.

As used herein, the term “CNS” can refer to the CNS as a whole or to particular parts of the CNS, e.g., the brain, the hypothalamus, the mediobasal hypothalamus, the ARC and/or the vagus nerve.

K_{ATP} channel activators, in particular diazoxide, are known to be effective in elevating blood glucose levels when administered to the periphery, and are used therapeutically for that purpose (see, e.g., U.S. Pat. No. 5,284,845). It is also known that K_{ATP} channel activators inhibit release of insulin, and have been evaluated for type 2 diabetes treatment because of that property (see, e.g., U.S. Pat. No. 6,197,765; Carr et al., 2003). However, the activation of K_{ATP} channels in the CNS has not previously been shown to be useful, particularly for lowering peripheral blood glucose, reducing hepatic gluconeogenesis, reducing triglycerides, reducing VLDL, reducing peripheral glucose production and/or for treating metabolic disorders such as diabetes (type 1 or type 2), hyperglycemia, insulin resistance, glucose intolerance, leptin resistance, metabolic syndrome, obesity, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, gonadotropin deficiency, amenorrhea and/or polycystic ovary syndrome).

Thus, in some embodiments, the invention is directed to methods of increasing K_{ATP} channel activity in the hypothalamus of a mammal. The methods comprise bringing a K_{ATP} channel activator into contact with the hypothalamus of the mammal in an amount effective to increase K_{ATP} activity in the hypothalamus. In particular embodiments, the methods comprise intranasally administering a K_{ATP} channel activator to mammal in an amount effective to increase K_{ATP} channel activity in the hypothalamus. Optionally, K_{ATP} channel activity is increased by at least about 10%, 15%, 20%, 25%, 30%, 35%, 40%, 45%, 50%, 60%, 75% or more.

K_{ATP} channel activity can be measured directly or indirectly by any means. For example, increases in K_{ATP} channel activity can be inferred as a result of administration of a K_{ATP} channel activator at a dose that has been previously determined to cause an increase in K_{ATP} channel activity, or that causes a measurable physiological response attributed to activation of hypothalamic K_{ATP} channels, for example increase in peripheral blood glucose levels, as described herein.

In particular embodiments, the mammal has a condition that is at least partially alleviated by an increase in hypothalamic K_{ATP} channel activation. Nonlimiting examples of such conditions include obesity, type 2 diabetes, type 1 diabetes, hyperglycemia, insulin resistance, glucose intolerance, leptin resistance, metabolic syndrome, gonadotropin deficiency, amenorrhea, polycystic ovary syndrome, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension or any combination thereof.

The invention is also directed to methods of reducing peripheral glucose levels (e.g., in blood, plasma or serum) in a mammal. The methods comprise administering a K_{ATP} activator to the CNS of the mammal in an amount effective to lower peripheral blood (or plasma or serum) glucose levels in the mammal. In representative embodiments, the methods comprise intranasally administering a K_{ATP} channel activator to the CNS of the mammal in an amount effective to lower peripheral blood (or plasma or serum) glucose levels in the mammal. Glucose levels can be measured by any means known in the art, e.g., as described herein.

As used herein, “reducing peripheral blood glucose levels” and similar terms refer to a statistically significant reduction. The reduction can be, for example, at least about a 5%, 10%, 15%, 20%, 25%, 30%, 35%, 40%, 45%, 50%, 60%, 75% reduction or more.

In representative embodiments, the mammal has a condition that is at least partially alleviated by a reduction in peripheral blood glucose levels, including but not limited to obesity, type 2 diabetes, type 1 diabetes, hyperglycemia, insulin resistance, glucose intolerance, leptin resistance, metabolic syndrome, gonadotropin deficiency, amenorrhea, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, polycystic ovary syndrome, or any combination thereof.

The invention is also directed to methods of reducing glucose production in a mammal. The methods comprise administering a K_{ATP} activator to the CNS of the mammal in an amount effective to reduce glucose production in the mammal. In particular embodiments, the methods comprise intranasally administering a K_{ATP} channel activator to the CNS of the mammal in an amount effective to reduce glucose production in the mammal. As used herein, the term “glucose production” can refer to whole animal glucose production, peripheral glucose production, or glucose production by particular organs or tissues (e.g., the liver and/or skeletal muscle). Glucose production can be determined by any method known in the art and as shown herein, e.g., by the pancreatic/insulin clamp technique.

In representative embodiments, glucose production is reduced by at least about 5%, 10%, 15%, 20%, 25%, 30%, 35%, 40%, 45%, 50%, 60%, 75% or more. In particular embodiments, glucose production is normalized (e.g., as compared with a suitable healthy control) in the subject.

In particular embodiments of this aspect of the invention, the mammal has a condition that is at least partially alleviated by a reduction in glucose production, including but not limited to obesity, type 2 diabetes, type 1 diabetes, hyperglycemia, insulin resistance, glucose intolerance, leptin resistance, metabolic syndrome, insulin resistance, gonadotropin deficiency, amenorrhea, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, polycystic ovary syndrome, or any combination of the foregoing.

Additionally, the present invention is directed to methods of reducing gluconeogenesis in the liver of a mammal. The methods comprise administering a K_{ATP} activator to the CNS of the mammal in an amount effective to reduce glucose production in the mammal. In particular embodiments, the methods comprise intranasally administering a K_{ATP} channel activator to the CNS of the mammal in an amount effective to reduce gluconeogenesis in the mammal. Optionally, the reduction can be at least about a 5%, 10%, 15%, 20%, 25%, 30%, 35%, 40%, 45%, 50%, 50%, 75% reduction or more. Gluconeogenesis can be measured by any means known in the art, e.g., as described herein.

In representative embodiments, the mammal has a condition that is at least partially alleviated by a reduction in gluconeogenesis, including but not limited to obesity, type 2 diabetes, type 1 diabetes, hyperglycemia, insulin resistance, glucose intolerance, leptin resistance, metabolic syndrome, insulin resistance, gonadotropin deficiency, amenorrhea, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, polycystic ovary syndrome, or any combination of the foregoing.

Particular embodiments of the invention are directed to methods of reducing serum triglyceride levels in a mammal. The methods comprise administering a K_{ATP} activator to the CNS of the mammal in an amount effective to reduce serum triglyceride levels in the mammal. In particular embodiments, the methods comprise intranasally administering a K_{ATP} channel activator to the CNS of the mammal in an amount effective to reduce serum triglyceride levels in the mammal. Serum triglyceride levels can be determined by any method known in the art.

In representative embodiments, serum triglyceride levels are reduced by at least about 5%, 10%, 15%, 20%, 25%, 30%, 35%, 40%, 45%, 50%, 60%, 75% or more. In particular embodiments, serum triglyceride levels are normalized (e.g., as compared with a suitable healthy control) in the subject. Elevated and normal ranges of triglycerides can be readily determined. In particular embodiments, normal levels of serum triglycerides are in the range of 70-150 mg/dl.

In particular embodiments of this aspect of the invention, the mammal has a condition that is at least partially alleviated by a reduction in serum triglyceride levels, including but not limited to obesity, type 2 diabetes, type 1 diabetes, hyperglycemia, insulin resistance, glucose intolerance, leptin resistance, metabolic syndrome, insulin resistance, gonadotropin deficiency, amenorrhea, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, polycystic ovary syndrome, or any combination of the foregoing.

Other representative embodiments of the invention are directed to methods of reducing VLDL levels in a mammal. The methods comprise administering a K_{ATP} activator to the CNS of the mammal in an amount effective to reduce serum VLDL levels in the mammal. In particular embodiments, the methods comprise intranasally administering a K_{ATP} channel activator to the CNS of the mammal in an amount effective to reduce serum VLDL levels in the mammal. Serum VLDL levels can be determined by any method known in the art.

In representative embodiments, serum VLDL levels are reduced by at least about 5%, 10%, 15%, 20%, 25%, 30%, 35%, 40%, 45%, 50%, 60%, 75% or more. In particular embodiments, serum VLDL levels are normalized (e.g., as compared with a suitable healthy control) in the subject. Elevated and normal ranges of VLDL can be readily determined. In particular embodiments, normal levels of serum VLDL are in the range of 20-40 mg/dl.

In particular embodiments of this aspect of the invention, the mammal has a condition that is at least partially alleviated by a reduction in serum VLDL levels, including but not limited to obesity, type 2 diabetes, type 1 diabetes, hyperglycemia, insulin resistance, glucose intolerance, leptin resistance, metabolic syndrome, insulin resistance, gonadotropin deficiency, amenorrhea, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, polycystic ovary syndrome, or any combination of the foregoing.

The invention further encompasses methods of treating metabolic disorders such as diabetes (e.g., type-1 and/or type-2 diabetes), metabolic syndrome, hyperglycemia, insulin resistance and/or glucose intolerance in a mammal by administering to the CNS of the mammal a K_{ATP} channel activator in an amount effective to treat the condition or disorder. The invention also provides methods of treating metabolic disorders such as diabetes (e.g., type-1 and/or type-2 diabetes), metabolic syndrome, hyperglycemia, insulin resistance and/or glucose intolerance in a mammal by intranasally administering to the CNS of the mammal a K_{ATP} channel activator in an amount effective to treat the condition or disorder. The invention can also be practiced to treat leptin resistance, gonadotropin deficiency, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, amenorrhea, and/or polycystic ovary syndrome by administration (e.g., intranasal administration) of a K_{ATP} channel activator to the CNS of the mammal in an amount effective to treat the condition or disorder.

As used herein, the term “diabetes” is intended to encompass both insulin dependent and non-insulin dependent (type I and type II, respectively) diabetes, unless one condition or the other is specifically indicated. Methods of diagnosing diabetes are well known in the art. In humans, diabetes is typically characterized as a fasting level of blood glucose greater than or equal to about 140 mg/dl or as a plasma glucose level greater than or equal to about 200 mg/dl as assessed at about two hours following the oral administration of a glucose load of about 75 g.

“Metabolic syndrome” is characterized by a group of metabolic risk factors in one person, including one or more of the following: central obesity (excessive fat tissue in and around the abdomen), atherogenic dyslipidemia (blood fat disorders—mainly high triglycerides and low HDL cholesterol—that foster plaque buildups in artery walls), raised blood pressure (e.g., 130/85 mmHg or higher), insulin resistance and/or glucose intolerance, a prothrombotic state (e.g., high fibrinogen or plasminogen activator inhibitor in the blood), and proinflammatory state (e.g., elevated high-sensitivity C-reactive protein in the blood). As used herein, the presence of metabolic syndrome in a subject can be diagnosed by any method currently known or later developed in the art. The criteria proposed by the Third Report of the National Cholesterol Education Program (NCEP) Expert Panel on Detection, Evaluation, and Treatment of High Blood Cholesterol in Adults (Adult Treatment Panel III) are the most widely used at this time to diagnose the metabolic syndrome. According to the ATP III criteria, the metabolic syndrome is identified by the presence of three or more of these components: central obesity as measured by waist circumference (men—greater than 40 inches; women—greater than 35 inches), fasting blood triglycerides greater than or equal to 150 mg/dL, blood HDL cholesterol (men—less than 40 mg/dl; women—less than 50 mg/dL), blood pressure greater than or equal to 130/85 mmHg, and fasting glucose greater than or equal to 110 mg/dL. The underlying causes of this syndrome are believed to be obesity, physical inactivity, and genetic factors. Subjects with metabolic syndrome are at increased risk of coronary heart disease, other diseases related to plaque buildup in artery walls (e.g., stroke and peripheral vascular disease) and/or type-2 diabetes. Metabolic syndrome has become increasingly common in the United States; as of October 2004, the American Heart Association estimates that about 47 million adults in the United States have metabolic syndrome.

Hyperglycemia is characterized by excessive blood (or plasma) glucose levels. Methods of diagnosing and evaluating hyperglycemia are known in the art. In general, fasting hyperglycemia is characterized by blood or plasma glucose concentration above the normal range after a subject has fasted for at least eight hours (e.g., the normal range is about 70-120 mg/dL). Postprandial hyperglycemia is generally characterized by blood or plasma glucose concentration above the normal range one to two hours after food intake by a subject.

By “insulin resistance” or “insulin insensitivity” it is meant a state in which a given level of insulin produces a less than normal biological effect (e.g., uptake of glucose). Insulin resistance is particularly prevalent in obese individuals or those with type-2 diabetes or metabolic syndrome. In type-2 diabetics, the pancreas is generally able to produce insulin, but there is an impairment in insulin action. As a result, hyperinsulinemia is commonly observed in insulin-resistant subjects. Insulin resistance is less common in type-I diabetics; although in some subjects, higher dosages of insulin have to be administered over time indicating the development of insulin resistance/insensitivity. The term “insulin resistance” or “insulin insensitivity” refers to whole animal insulin resistance/insensitivity unless specifically indicated otherwise. Methods of evaluating insulin resistance/insensitivity are known in the art, for example, hyperinsulinemic/euglycemic clamp studies, insulin tolerance tests, uptake of labeled glucose and/or incorporation into glycogen in response to insulin stimulation, and measurement of known components of the insulin signaling pathway.

“Glucose intolerance” is characterized by an impaired ability to maintain blood (or plasma) glucose concentrations following a glucose load (e.g., by ingestion or infusion) resulting in hyperglycemia. Glucose intolerance is generally indicative of an insulin deficiency or insulin resistance. Methods of evaluating glucose tolerance/intolerance are known in the art, e.g., the oral glucose tolerance test.

Any degree of obesity can be treated, and the inventive methods can be practiced for research, cosmetic and/or medical purposes. In particular embodiments, the subject is at least about 5%, 10%, 20%, 30%, 50, 75% or even 100% or greater over normal body weight. Methods of determining normal body weight are known in the art. For example, in humans, normal body weight can be defined as a BMI index of 18.5-24.9 kg/meter² (NHLBI (National Heart Lung and Blood Institute) Obesity Education Initiative. The Practical Guide—Identification, Evaluation and Treatment of Overweight and Obesity in Adults. NIH Publication No. 004084 (2000); obtainable at http://www.nhlbi.nih.gov/guidelines/obesitv/prctqd_b.pdf). In particular embodiments, the invention is practiced to treat subjects having a BMI index of about 24.9 kg/meter² or greater. In representative embodiments, the methods of the invention result in at least about a 5%, 10%, 20%, 30%, 50% or greater reduction in degree of obesity (e.g., as determined by kg of weight loss or by reduction in BMI).

The foregoing methods of the invention can be practiced with any suitable K_{ATP} channel activator now known or later discovered. Illustrative examples of K_{ATP} channel activators are described herein.

The inventors have also discovered that inhibiting hypothalamic K_{ATP} channels effectively causes an increase in glucose production and peripheral glucose levels (e.g., in blood, plasma or serum) in the mammal. See Example. Thus, in further embodiments, the invention is directed to methods of increasing glucose production and/or peripheral blood (or plasma or serum) glucose levels in a mammal. The methods comprise administering a K_{ATP} inhibitor to the CNS of the mammal in an amount effective to increase glucose production and/or peripheral blood (or plasma or serum) glucose levels in the mammal. In representative embodiments, the methods comprise intranasally administering a K_{ATP} channel inhibitor to the CNS of a mammal in an amount effective to increase glucose production and/or peripheral blood (or plasma or serum) glucose levels in the mammal. As used herein, an “increase in glucose production” or “increase in peripheral blood glucose levels” (or like terms) is any amount of glucose production or peripheral blood glucose that is significantly higher than the amount measured before treatment. The increase can be at least about 5%, 10%, 15%, 20%, 25%, 30%, 35%, 40%, 45% or more. In particular embodiments, glucose production and/or peripheral blood (or plasma or serum) glucose levels are normalized (e.g., as compared with a suitable healthy control) in the subject.

According to particular embodiments, the mammal has a condition that is at least partially alleviated by increasing glucose production, including but not limited to hypoglycemia. The mammal can be undergoing a treatment that causes insufficient food intake, appetite and/or glucose production, such as cachexia or cancer chemotherapy. Further, the mammal can have a viral infection that causes insufficient glucose production, such as HIV-1 infection.

These aspects of the invention can be practiced with any suitable K_{ATP} channel inhibitor now known or later discovered. Illustrative examples of K_{ATP} channel inhibitory compounds are described herein.

As used herein, an “effective amount” refers to an amount of a compound or pharmaceutical composition that is sufficient to produce a desired effect, which is optionally a therapeutic effect (i.e., by administration of a therapeutically effective amount). For example, an “effective amount” can be an amount that is sufficient to activate or inhibit K_{ATP} channels in the CNS, to reduce glucose production, to reduce blood glucose levels, to reduce gluconeogenesis, to treat metabolic disorders such as metabolic syndrome, hyperglycemia, glucose intolerance, insulin resistance, diabetes (e.g., type-1 or type-2 diabetes), and/or obesity and/or to treat leptin resistance, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, to treat gonadotropin deficiency, amenorrhea and/or polycystic ovary syndrome.

A “therapeutically effective” amount as used herein is an amount that provides some improvement or benefit to the subject. Alternatively stated, a “therapeutically effective” amount is an amount that provides some alleviation, mitigation, delay and/or decrease in at least one clinical symptom and/or prevent the onset or progression of at least one clinical symptom. Clinical symptoms associated with the disorders that can be treated by the methods of the invention are well-known to those skilled in the art. Further, those skilled in the art will appreciate that the therapeutic effects need not be complete or curative, as long as some benefit is provided to the subject.

By the terms “treat,” “treating” or “treatment of” (or grammatically equivalent terms) it is meant that the severity of the subject's condition is reduced or at least partially improved or ameliorated and/or that some alleviation, mitigation or decrease in at least one clinical symptom is achieved and/or there is a delay in the progression of the condition and/or prevention or delay of the onset of a disease or illness. Thus, the terms “treat,” “treating” or “treatment of” (or grammatically equivalent terms) refer to both prophylactic and therapeutic treatment regimes.

The present invention can also be used to screen or identify compounds that can be administered to modulate (e.g., activate or inhibit) K_{ATP} channels in the CNS, to reduce glucose production, to reduce blood glucose levels, to reduce gluconeogenesis, to treat metabolic disorders such as metabolic syndrome, hyperglycemia, glucose intolerance, insulin resistance, diabetes (e.g., type-1 or type-2 diabetes) and/or obesity, and/or to treat leptin resistance, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, to treat gonadotropin deficiency, amenorrhea and/or polycystic ovary syndrome. Subjects for use in the screening methods of the invention are as described above.

For example, in particular embodiments, a compound is delivered to a subject and hypothalamic (e.g., ARC) K_{ATP} channel activity is evaluated. An elevation in K_{ATP} channel activity in the hypothalamus indicates that the compound is a compound that can be administered to activate K_{ATP} channels in the CNS. Optionally, elevation in K_{ATP} channel activity is evaluated by comparison with a suitable control.

As another non-limiting example, the invention provides a method of identifying a compound that can be delivered to the CNS of a subject (e.g., by direct administration to the hypothalamus or brain) to reduce glucose production, to reduce glucose levels, to reduce gluconeogenesis, to reduce serum triglycerides, to reduce serum VLDL, to treat metabolic disorders such as metabolic syndrome, hyperglycemia, glucose intolerance, insulin resistance, diabetes (e.g., type-1 or type-2 diabetes) and/or obesity, and/or to treat leptin resistance, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, to treat gonadotropin deficiency, amenorrhea and/or polycystic ovary syndrome. In particular embodiments, elevation in K_{ATP} channel activity is evaluated by comparison with a suitable control.

As a further non-limiting example, the invention provides a method of identifying a compound that can be delivered to the CNS of a subject (e.g., by direct administration to the hypothalamus or brain) to treat diabetes. In a representative embodiment, a compound is administered to the CNS a subject and K_{ATP} channel activity in the CNS is determined. An elevation in K_{ATP} channel activity in the CNS indicates that the compound is a compound that can be administered to the CNS to treat diabetes. Optionally, elevation in K_{ATP} channel activity is evaluated by comparison with a suitable control.

The invention further provides a method of identifying a compound that can be delivered to the CNS of a subject (e.g., by direct administration to the hypothalamus or brain) to treat metabolic syndrome. In a representative embodiment, a compound is administered to the CNS of a subject and the level of K_{ATP} channel activity in the CNS is determined. An elevation in K_{ATP} channel activity in the CNS indicates that the compound is a compound that can be administered to the CNS to treat metabolic syndrome. Optionally, elevation in K_{ATP} channel activity is evaluated by comparison with a suitable control.

In yet other representative embodiments, the methods of the invention are practiced to identify a compound that can be delivered to the CNS of a subject (e.g., by direct administration to the hypothalamus or brain) to treat obesity. In a representative embodiment, a compound is administered to the CNS of a subject and the level of K_{ATP} channel activity in the CNS is determined. An elevation in K_{ATP} channel activity in the CNS indicates the compound is a compound that can be administered to the CNS to treat obesity. Optionally, elevation in K_{ATP} channel activity is evaluated by comparison with a suitable control.

The methods above can be modified to identify compounds that can be delivered to the CNS to inhibit K_{ATP} channel activity, increase blood glucose levels, to increase glucose production and/or increase gluconeogenesis.

The present invention can also be used to screen or identify compounds that can be administered intranasally to modulate (e.g., activate or inhibit) K_{ATP} channels in the CNS, to reduce glucose production, to reduce blood glucose levels, to reduce gluconeogenesis, to reduce serum triglycerides, to reduce serum VLDL, to treat metabolic disorders such as metabolic syndrome, hyperglycemia, glucose intolerance, insulin resistance, diabetes (e.g., type-1 or type-2 diabetes) and/or obesity, and/or to treat leptin resistance, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, to treat gonadotropin deficiency, amenorrhea and/or polycystic ovary syndrome. Subjects for use in the screening methods of the invention are as described above.

For example, in particular embodiments, a compound is delivered by intranasal administration to a subject and hypothalamic (e.g., ARC) K_{ATP} channel activity is evaluated. An elevation in K_{ATP} channel activity in the hypothalamus indicates that the compound is a compound that can be administered intranasally to activate K_{ATP} channels in the CNS. Optionally, elevation in K_{ATP} channel activity is evaluated by comparison with a suitable control.

As another non-limiting example, the invention provides a method of identifying a compound that can be delivered by intranasal administration to a subject to reduce glucose production, reduce glucose levels, reduce gluconeogenesis, to reduce serum triglycerides, to reduce serum VLDL and/or to treat hyperglycemia, insulin resistance and/or glucose intolerance. In exemplary embodiments, a compound is administered intranasally to a subject and the levels of K_{ATP} channel activity in the CNS are determined. An elevation in K_{ATP} channel activity in the CNS indicates that the compound is a compound that can be administered intranasally to reduce glucose production, to reduce blood glucose levels, to reduce gluconeogenesis and/or to treat hyperglycemia, insulin resistance and/or glucose intolerance. In particular embodiments, elevation in K_{ATP} channel activity is evaluated by comparison with a suitable control.

As a further non-limiting example, the invention provides a method of identifying a compound that can be delivered by intranasal administration to a subject to treat diabetes. In a representative embodiment, a compound is administered intranasally to a subject and K_{ATP} channel activity in the CNS is determined. An elevation in K_{ATP} channel activity in the CNS indicates that the compound is a compound that can be administered intranasally to treat diabetes. Optionally, elevation in K_{ATP} channel activity is evaluated by comparison with a suitable control.

The invention further provides a method of identifying a compound that can be delivered by intranasal administration to a subject to treat metabolic syndrome. In a representative embodiment, a compound is administered intranasally to a subject and the level of K_{ATP} channel activity in the CNS is determined. An elevation in K_{ATP} channel activity in the CNS indicates that the compound is a compound that can be administered intranasally to treat metabolic syndrome. Optionally, elevation in K_{ATP} channel activity is evaluated by comparison with a suitable control.

In yet other representative embodiments, the methods of the invention are practiced to identify a compound that can be delivered by intranasal administration to a subject to treat obesity. In a representative embodiment, a compound is administered intranasally to a subject and the level of K_{ATP} channel activity in the CNS is determined. An elevation in K_{ATP} channel activity in the CNS indicates the compound is a compound that can be administered intranasally to treat obesity. Optionally, elevation in K_{ATP} channel activity is evaluated by comparison with a suitable control.

The methods above can be modified to identify compounds that can be delivered by intranasal administration to the CNS to inhibit K_{ATP} channel activity, to increase blood glucose levels, to increase glucose production and/or increase gluconeogenesis.

K_{ATP} Channel Activators and Inhibitors.

Examples of compounds that can activate or inhibit K_{ATP} channels include small organic molecules, oligomers, polypeptides (including enzymes, antibodies and antibody fragments), carbohydrates, lipids, coenzymes, nucleic acids (including DNA, RNA and chimerics and analogues thereof), nucleic acid mimetics, nucleotides, nucleotide analogs, as well as other molecules (e.g., cytokines or enzyme inhibitors) that directly or indirectly inhibit molecules that activate or inhibit K_{ATP} channels. The compound can be an activator or inhibitor of one or more K_{ATP} channel types (e.g., Kir6.1/SUR1 or Kir6.2/SUR1), as described above.

As used herein, a “small organic molecule” is an organic molecule of generally less than about 2000 MW that is not an oligomer. Small non-oligomeric organic compounds include a wide variety of organic molecules, such as heterocyclics, aromatics, alicyclics, aliphatics and combinations thereof, comprising steroids, antibiotics, enzyme inhibitors, ligands, hormones, drugs, alkaloids, opioids, terpenes, porphyrins, toxins, catalysts, as well as combinations thereof.

Oligomers include oligopeptides, oligonucleotides, oligosaccharides, polylipids, polyesters, polyamides, polyurethanes, polyureas, polyethers, and poly (phosphorus derivatives), e.g. phosphates, phosphonates, phosphoramides, phosphonamides, phosphites, phosphinamides, etc., poly (sulfur derivatives) e.g., sulfones, sulfonates, sulfites, sulfonamides, sulfenamides, etc., where for the phosphorous and sulfur derivatives the indicated heteroatom are optionally bonded to C, H, N, O or S, and combinations thereof.

The invention can be practiced with any K_{ATP} channel activator now known or later discovered. Many K_{ATP} channel activators are known in the art. Nonlimiting examples of K_{ATP} channel activators include the various known substituted guanidines (such as cyanoguanidines) and benzothiazine 1,1-dioxide K_{ATP} channel activators and fused 1,2,4-thiadiazine and fused 1,4-thisazine derivative K_{ATP} channel activators, and include diazoxide, pinacidil, (−)-cromakalim, aprikalim, bimakalim, emakalim, nicordandil, NNC 55-0118, NN414, EMD55387, HOE234, KRN2391, diaminonitroethane, minoxidil sulfate, P1060, P1075, RP49356, RP66471, and any combination thereof (see, e.g., U.S. Pats. No. 6,147,098 and 5,889,002; Carr et al., 2003; Pijewski & Kotliński, 2000). The K_{ATP} channel activator can further be an arylcyanoguanidine such as a phenylcyanoguanidine substituted with lipophilic electron-withdrawing functional groups (e.g., N-cyano-N′[3,5-bis-(trifluoromethyl)phenyl]-N″-(cyclopentyl)guanidine and N-cyano-N′-(3,5,-dichlorophenyl)-N-(3-methylbutyl)guanidine (see, e.g., Tagmose et al., (2004) J. Med. Chem. 47:3202-11). The K_{ATP} channel activator can also be 2-(4-methoxyphenoxy)-5-nitro-N-(4-sulfamoylphenyl)benzamide or an analog thereof (see, e.g., Nielsen et al., (2004) Bioorg. Med. Chem. Lett. 14:5727-30). In particular embodiments, the K_{ATP} channel activator is diazoxide, which is known to have low mammalian toxicity.

Additional nonlimiting examples of compounds that activate K_{ATP} channels are shown in Table 1.

The activators can also be pro-drugs that are converted to the active compound in vivo. Further, the activators can be modified to increase their lipophilicity and/or absorption across cell membranes or the nasal mucosa, e.g., by conjugation with lipophilic moieties such as fatty acids.

As another option, nucleic acids encoding K_{ATP} channel protein(s) can be delivered to the CNS to increase K_{ATP} channel activity in the CNS. Nucleic acid sequences encoding K_{ATP} channel protein are known in the art and can be delivered using any suitable method.

The invention also encompasses methods of inhibiting K_{ATP} channel activity in the CNS. This aspect of the invention can be practiced with any K_{ATP} channel inhibitor now known or later discovered. K_{ATP} channel inhibitors are known in the art. Nonlimiting examples of K_{ATP} channel inhibitors include the various known sulfonylurea inhibitors and include the inhibitors glibenclamide, phentolamine, ciclazindol, lidocaine, glipizide, U37883A, tolbutamide, and any combination thereof. In particular embodiments, the K_{ATP} channel inhibitor is glibenclamide.

Other nonlimiting of K_{ATP} channel inhibitors are shown in Table I.

The inhibitors can be pro-drugs that are converted to the active compound in vivo. Further, the inhibitors can be modified to increase their lipophilicity and/or absorption across cell membranes or the nasal mucosa, e.g., by conjugation with lipophilic moieties such as fatty acids.

In particular embodiments of the invention, the compound is an antibody or antibody fragment that binds to a K_{ATP} channel protein and reduces the activity thereof. The antibody or antibody fragment is not limited to any particular form and can be a polyclonal, monoclonal, bispecific, humanized, chimerized antibody or antibody fragment and can further be a Fab fragment, single chain antibody, and the like.

In other embodiments, the compound is an inhibitory nucleic acid such as an interfering RNA (RNAi) including short interfering RNAs (siRNA), an antisense nucleic acid, a ribozyme or a nucleic acid mimetic that reduces K_{ATP} channel expression.

K_{ATP} channel proteins and nucleic acids encoding the same are known in the art, and can be used to facilitate the production of antibodies and inhibitory nucleic acids.

Methods of Administration to the CNS.

The blood-brain barrier presents a barrier to the passive diffusion of substances from the bloodstream into various regions of the CNS. However, active transport of certain agents is known to occur in either direction across the blood-brain barrier. Substances that may have limited access to the brain from the bloodstream can be injected directly into the cerebrospinal fluid. Cerebral ischemia and inflammation are also known to modify the blood-brain barrier and result in increased access to substances in the bloodstream.

Administration of a therapeutic compound directly to the brain is known in the art. Intrathecal injection administers agents directly to the brain ventricles and the spinal fluid. Surgically-implantable infusion pumps are available to provide sustained administration of agents directly into the spinal fluid. Lumbar puncture with injection of a pharmaceutical compound into the cerebrospinal fluid (“spinal injection”) is known in the art, and is suited for administration of compounds and compositions according to the present invention. In particular embodiments, intracerebroventricular (ICV) administration is used to deliver the compound (e.g., ICV injection through a surgically implanted cannulae). According to this embodiment, the ICV administration can be to the third cerebral ventricle of the brain. Thus, in representative embodiments, the K_{ATP} activator or inhibitor can be administered directly to the brain of the mammal, e.g., by direct injection or through a pump.

Alternatively, the K_{ATP} activator or inhibitor can be administered peripherally in a manner that permits the activator to cross the blood-brain barrier of the mammal sufficiently to activate hypothalamic K_{ATP}. With any mode of administration, the K_{ATP} activator or inhibitor can be formulated in a pharmaceutically acceptable excipient.

By “pharmaceutically acceptable” it is meant a material that (i) is compatible with the other ingredients of the composition without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include, without limitation, any of the standard pharmaceutical carriers such as phosphate buffered saline solutions, water, emulsions such as oil/water emulsions, microemulsions, and the like.

Further, the K_{ATP} channel activator or inhibitor compounds can be formulated in a pharmaceutical composition that enhances the ability of the compound(s) to cross the blood-brain barrier of the mammal. Pharmacologic-based procedures are known in the art for circumventing the blood brain barrier, including the conversion of hydrophilic compounds into lipid-soluble drugs. For example, the active compound can be encapsulated in a lipid vesicle or liposome.

The intra-arterial infusion of hypertonic substances to transiently open the blood-brain barrier and allow passage of hydrophilic drugs into the brain is also known in the art. U.S. Pat. No. 5,686,416 to Kozarich et al. discloses the co-administration of receptor mediated permeabilizer (RMP) peptides with therapeutic compounds to be delivered to the interstitial fluid compartment of the brain, to cause an increase in the permeability of the blood-brain barrier and effect increased delivery of the therapeutic compounds to the brain. Intravenous or intraperitoneal administration may also be used in practicing the present invention.

One method of transporting an active agent across the blood-brain barrier is to couple or conjugate the active compound to a second molecule (a “carrier”), which is a peptide or non-proteinaceous moiety selected for its ability to penetrate the blood-brain barrier and transport the active agent across the blood-brain barrier. Examples of suitable carriers include pyridinium, fatty acids, inositol, cholesterol, and glucose derivatives. The carrier may be a compound that enters the brain through a specific transport system in brain endothelial cells. Chimeric peptides adapted for delivering neuropharmaceutical agents into the brain by receptor-mediated transcytosis through the blood-brain barrier are disclosed in U.S. Pat. No. 4,902,505 to Pardridge et al. These chimeric peptides comprise a pharmaceutical agent conjugated with a transportable peptide capable of crossing the blood-brain barrier by transcytosis. Specific transportable peptides disclosed by Pardridge et al. include histone, insulin, transferrin, and others. Conjugates of a compound with a carrier molecule, to cross the blood-brain barrier, are also disclosed in U.S. Pat. No. 5,604,198 to Poduslo et al. Specific carrier molecules disclosed include hemoglobin, lysozyme, cytochrome c, ceruloplasmin, calmodulin, ubiquitin and substance P. See also U.S. Pat. No. 5,017,566 to Bodor.

The K_{ATP} channel activator or inhibitor compositions can be formulated without undue experimentation for administration to a mammal, including humans, as appropriate for the particular application. Additionally, proper dosages of the compositions can be determined without undue experimentation using standard dose-response protocols.

Accordingly, the compositions designed for oral, lingual, sublingual, buccal and intrabuccal administration can be made without undue experimentation by means well known in the art, for example with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, cornstarch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

The compositions of the present invention can easily be administered parenterally such as for example, by intravenous, intramuscular, intrathecal or subcutaneous injection. Parenteral administration can be accomplished by incorporating the compositions of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the pharmaceutical K_{ATP} activator or inhibitor compositions into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches (such as the well-known nicotine patch), ointments, creams, gels, salves and the like.

Where the K_{ATP} activator or inhibitor is administered peripherally such that it crosses the blood-brain barrier, the K_{ATP} activator or inhibitor can be formulated in a pharmaceutical composition that enhances the ability of the activator to cross the blood-brain barrier of the mammal. Such formulations are known in the art and include lipophilic compounds to promote absorption. Uptake of non-lipophilic compounds can be enhanced by combination with a lipophilic substance. Lipophilic substances that can enhance delivery of the compound across the nasal mucus include but are not limited to fatty acids (e.g., palmitic acid), gangliosides (e.g., GM-I), phospholipids (e.g., phosphatidylserine), and emulsifiers (e.g., polysorbate 80), bile salts such as sodium deoxycholate, and detergent-like substances including, for example, polysorbate 80 such as Tween™, octoxynol such as Triton™ X-100, and sodium tauro-24,25-dihydrofusidate (STDHF). See Lee et al., Biopharm., April 1988 issue: 3037.

In particular embodiments of the invention, the K_{ATP} activator or inhibitor is combined with micelles comprised of lipophilic substances. Such micelles can modify the permeability of the nasal membrane to enhance absorption of the compound. Suitable lipophilic micelles include without limitation gangliosides (e.g., GM-1 ganglioside), and phospholipids (e.g., phosphatidylserine). Bile salts and their derivatives and detergent-like substances can also be included in the micelle formulation. The active compound can be combined with one or several types of micelles, and can further be contained within the micelles or associated with their surface.

Alternatively, the active compound can be combined with liposomes (lipid vesicles) to enhance absorption. The active compound can be contained or dissolved within the liposome and/or associated with its surface. Suitable liposomes include phospholipids (e.g., phosphatidylserine) and/or gangliosides (e.g., GM-1). For methods to make phospholipid vesicles, see for example, U.S. Pat. No. 4,921,706 to Roberts et al., and U.S. Pat. No. 4,895,452 to Yiournas et al. Bile salts and their derivatives and detergent-like substances can also be included in the liposome formulation.

Other methods of delivering compounds across the blood-brain barrier are well-known in the art. Methods of intranasal delivery to the CNS are discussed in more detail below.

Pharmaceutical Formulations and Modes of Intranasal Delivery.

The invention also encompasses pharmaceutical compositions formulated for intranasal administration comprising one or more K_{ATP} channel activators or inhibitors in a pharmaceutically acceptable carrier. The one or more compounds can individually be prodrugs that are converted to the active compound in vivo. In particular embodiments, the invention provides a pharmaceutical composition formulated for intranasal administration comprising one or more K_{ATP} channel activators or inhibitors that activates or inhibits, respectively, K_{ATP} channels in the CNS. K_{ATP} channel activators and inhibitors are known in the art and are discussed in more detail hereinabove.

By “pharmaceutically acceptable” it is meant a material that (i) is compatible with the other ingredients of the composition without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Examples of pharmaceutically acceptable carriers include, without limitation, any of the standard pharmaceutical carriers such as phosphate buffered saline solutions, water, emulsions such as oil/water emulsions, microemulsions, and the like.

The formulations of the invention can optionally comprise medicinal agents, pharmaceutical agents, carriers, dispersing agents, diluents, humectants, wetting agents, thickening agents, odorants, humectants, penetration enhancers, preservatives, and the like.

The compositions of the invention can be formulated for intranasal administration in a pharmaceutical carrier in accordance with known techniques. See, e.g., Remington, The Science And Practice of Pharmacy (20^{th} edition, 2000). Suitable nontoxic pharmaceutically acceptable nasal carriers will be apparent to those skilled in the art of nasal pharmaceutical formulations (see, e.g., Remington's Pharmaceutical Sciences (Maack Publishing Co., Easton latest edition). Further, it will be understood by those skilled in the art that the choice of suitable carriers, absorption enhancers, humectants, adhesives, etc., will typically depend on the nature of the active compound and the particular nasal formulation, for example, a nasal solution (e.g., for use as drops, spray or aerosol), a nasal suspension, a nasal ointment, a nasal gel, or another nasal formulation. Aerosols are discussed in more detail in the following section.

The carrier can be a solid or a liquid, or both, and is optionally formulated with the composition as a unit-dose formulation. Such dosage forms can be powders, solutions, suspensions, emulsions and/or gels. With respect to solutions or suspensions, dosage forms can be comprised of micelles of lipophilic substances, liposomes (phospholipid vesicles/membranes), and/or a fatty acid (e.g., palmitic acid). In particular embodiments, the pharmaceutical composition is a solution or suspension that is capable of dissolving in the fluid secreted by mucous membranes of the olfactory epithelium, which can advantageously enhance absorption.

The pharmaceutical composition can be an aqueous solution, a nonaqueous solution or a combination of an aqueous and nonaqueous solution.

Suitable aqueous solutions include but are not limited to aqueous gels, aqueous suspensions, aqueous microsphere suspensions, aqueous microsphere dispersions, aqueous liposomal dispersions, aqueous micelles of liposomes, aqueous microemulsions, and any combination of the foregoing, or any other aqueous solution that can dissolve in the fluid secreted by the mucosal membranes of the nasal cavity. Exemplary nonaqueous solutions include but are not limited to nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing, or any other nonaqueous solution that can dissolve or mix in the fluid secreted by the mucosal membranes of the nasal cavity.

Examples of powder formulations include without limitation simple powder mixtures, micronized powders, powder microspheres, coated powder microspheres, liposomal dispersions, and any combination of the foregoing. Powder microspheres can be formed from various polysaccharides and celluloses, which include without limitation starch, methylcellulose, xanthan gum, carboxymethylcellulose, hydroxypropyl cellulose, carbomer, alginate polyvinyl alcohol, acacia, chitosans, and any combination thereof.

In particular embodiments, the compound is one that is at least partially, or even substantially (e.g., at least 80%, 90%, 95% or more) soluble in the fluids that are secreted by the nasal mucosa (e.g., the mucosal membranes that surround the cilia of the olfactory receptor cells of the olfactory epithelium) so as to facilitate absorption. Alternatively or additionally, the compound can be formulated with a carrier and/or other substances that foster dissolution of the agent within nasal secretions, including without limitation fatty acids (e.g., palmitic acid), gangliosides (e.g., GM-I), phospholipids (e.g., phosphatidylserine, and emulsifiers (e.g., polysorbate 80).

Optionally, drug solubilizers can be included in the pharmaceutical composition to improve the solubility of the compound and/or to reduce the likelihood of disruption of nasal membranes which can be caused by application of other substances, for example, lipophilic odorants. Suitable solubilizers include but are not limited to amorphous mixtures of cyclodextrin derivatives such as hydroxypropylcylodextrins (see, for example, Pitha et al., (1988) Life Sciences 43:493-502).

In representative embodiments, the compound is lipophilic to promote absorption. Uptake of non-lipophilic compounds can be enhanced by combination with a lipophilic substance. Lipophilic substances that can enhance delivery of the compound across the nasal mucus include but are not limited to esters, fatty acids (e.g., palmitic acid), gangliosides (e.g., GM-I), phospholipids (e.g., phosphatidylserine), and emulsifiers (e.g., polysorbate 80), bile salts such as sodium deoxycholate, and detergent-like substances including, for example, polysorbate 80 such as Tween™, octoxynol such as Triton™ X-100, and sodium tauro-24,25-dihydrofusidate (STDHF). See Lee et al., Biopharm., April 1988 issue: 3037.

In particular embodiments of the invention, the active compound is combined with micelles comprised of lipophilic substances. Such micelles can modify the permeability of the nasal membrane to enhance absorption of the compound. Suitable lipophilic micelles include without limitation gangliosides (e.g., GM-1 ganglioside), and phospholipids (e.g., phosphatidylserine). Bile salts and their derivatives and detergent-like substances can also be included in the micelle formulation. The active compound can be combined with one or several types of micelles, and can further be contained within the micelles or associated with their surface.

Alternatively, the active compound can be combined with liposomes (lipid vesicles) to enhance absorption. The active compound can be contained or dissolved within the liposome and/or associated with its surface. Suitable liposomes include phospholipids (e.g., phosphatidylserine) and/or gangliosides (e.g., GM-1). For methods to make phospholipid vesicles, see for example, U.S. Pat. No. 4,921,706 to Roberts et al., and U.S. Pat. No. 4,895,452 to Yiournas et al. Bile salts and their derivatives and detergent-like substances can also be included in the liposome formulation.

In representative embodiments, the pH of the pharmaceutical composition ranges from about 2, 3, 3.5 or 5 to about 7, 8 or 10. Exemplary pH ranges include without limitation from about 2 to 8, from about 3.5 to 7, and from about 5 to 7. Those skilled in the art will appreciate that because the volume of the pharmaceutical composition administered is generally small, nasal secretions may alter the pH of the administered dose, since the range of pH in the nasal cavity can be as wide as 5 to 8. Such alterations can affect the concentration of un-ionized drug available for absorption. Accordingly, in representative embodiments, the pharmaceutical composition further comprises a buffer to maintain or regulate pH in situ. Typical buffers include but are not limited to acetate, citrate, prolamine, carbonate and phosphate buffers.

In embodiments of the invention, the pH of the pharmaceutical composition is selected so that the internal environment of the nasal cavity after administration is on the acidic to neutral side, which (1) can provide the active compound in an un-ionized form for absorption, (2) prevents growth of pathogenic bacteria in the nasal passage that is more likely to occur in an alkaline environment, and (3) reduces the likelihood of irritation of the nasal mucosa.

Further, in particular embodiments, the net charge on the compound is a positive or neutral charge.

According to other embodiments of the invention, the compound has a molecular weight of about 50 kilodaltons or less, 10 kilodaltons or less, 5 kilodaltons or less, 2 kilodaltons or less, 1 kilodalton or less, or 500 daltons or less.

For liquid and powder sprays or aerosols, the pharmaceutical composition can be formulated to have any suitable and desired particle size. In illustrative embodiments, the majority and/or the mean size of the particles or droplets range in size from equal to or greater than about 1, 2.5, 5, 10, 15 or 20 microns and/or equal to or less than about 25, 30, 40, 50, 60 or 75 microns. Representative examples of suitable ranges for the majority and/or mean particle or droplet size include, without limitation, from about 5 to 50 microns, from about 20 to 50 microns, and from about 15 to 30 microns, which facilitate the deposition of an effective amount of the active compound in the nasal cavity (e.g., in the olfactory region and/or in the sinus region). In general, particles or droplets smaller than about 5 microns will be deposited in the trachea or even the lung, whereas particles or droplets that are about 50 microns or larger generally do not reach the nasal cavity and are deposited in the anterior nose.

In particular embodiments, the pharmaceutical composition is isotonic to slightly hypertonic, e.g., having an osmolarity ranging from about 150 to 550 mOsM. As another particular example, the pharmaceutical composition is isotonic having, e.g., an osmolarity ranging from approximately 150 to 350 mOsM.

According to particular methods of intranasal delivery, it can be desirable to prolong the residence time of the pharmaceutical composition in the nasal cavity (e.g., in the olfactory region and/or in the sinus region), for example, to enhance absorption. Thus, the pharmaceutical composition can optionally be formulated with a bioadhesive polymer, a gum (e.g., xanthan gum), chitosan (e.g., highly purified cationic polysaccharide), pectin (or any carbohydrate that thickens like a gel or emulsifies when applied to nasal mucosa), a microsphere (e.g., starch, albumin, dextran, cyclodextrin), gelatin, a liposome, carbamer, polyvinyl alcohol, alginate, acacia, chitosans and/or cellulose (e.g., methyl or propyl; hydroxyl or carboxy; carboxymethyl or hydroxylpropyl), which are agents that enhance residence time in the nasal cavity. As a further approach, increasing the viscosity of the dosage formulation can also provide a means of prolonging contact of agent with nasal epithelium. The pharmaceutical composition can be formulated as a nasal emulsion, ointment or gel, which offer advantages for local application because of their viscosity.

Moist and highly vascularized membranes can facilitate rapid absorption; consequently, the pharmaceutical composition can optionally comprise a humectant, particularly in the case of a gel-based composition so as to assure adequate intranasal moisture content. Examples of suitable humectants include but are not limited to glycerin or glycerol, mineral oil, vegetable oil, membrane conditioners, soothing agents, and/or sugar alcohols (e.g., xylitol, sorbitol; and/or mannitol). The concentration of the humectant in the pharmaceutical composition will vary depending upon the agent selected and the formulation.

The pharmaceutical composition can also optionally include an absorption enhancer, such as an agent that inhibits enzyme activity, reduces mucous viscosity or elasticity, decreases mucociliary clearance effects, opens tight junctions, and/or solubilizes the active compound. Chemical enhancers are known in the art and include chelating agents (e.g., EDTA), fatty acids, bile acid salts, surfactants, and/or preservatives. Enhancers for penetration can be particularly useful when formulating compounds that exhibit poor membrane permeability, lack of lipophilicity, and/or are degraded by aminopeptidases. The concentration of the absorption enhancer in the pharmaceutical composition will vary depending upon the agent selected and the formulation.

To extend shelf life, preservatives can optionally be added to the pharmaceutical composition. Suitable preservatives include but are not limited to benzyl alcohol, parabens, thimerosal, chlorobutanol and benzalkonium chloride, and combinations of the foregoing. The concentration of the preservative will vary depending upon the preservative used, the compound being formulated, the formulation, and the like. In representative embodiments, the preservative is present in an amount of about 2% by weight or less.

The pharmaceutical composition can optionally contain an odorant, e.g., as described in EP 0 504 263 B1 to provide a sensation of odor, to aid in inhalation of the composition so as to promote delivery to the olfactory region and/or to trigger transport by the olfactory neurons.

As another option, the composition can comprise a flavoring agent, e.g., to enhance the taste and/or acceptability of the composition to the subject.

The invention also encompasses methods of intranasal administration of the pharmaceutical formulations of the invention. In particular embodiments, the pharmaceutical composition is delivered to the upper third of the nasal cavity, the olfactory region and/or the sinus region of the nose. The olfactory region is a small area that is typically about 2-10 cm² in man (25 cm² in the cat) located in the upper third of the nasal cavity for deposition and absorption by the olfactory epithelium and subsequent transport by olfactory receptor neurons. Located on the roof of the nasal cavity, the olfactory region is desirable for delivery because it is the only known part of the body in which an extension of the CNS comes into contact with the environment (Bois et al., Fundamentals of Otolaryngology, p. 184, W.B. Saunders Co., Phila., 1989).

In particular embodiments, the pharmaceutical composition is administered to the subject in an effective amount, optionally, a therapeutically effective amount (each as described hereinabove) Dosages of pharmaceutically active compositions can be determined by methods known in the art, see, e.g., Remington's Pharmaceutical Sciences (Maack Publishing Co., Easton, Pa.; 18^{th} edition, 1990).

A therapeutically effective amount will vary with the age and general condition of the subject, the severity of the condition being treated, the particular compound or composition being administered, the duration of the treatment, the nature of any concurrent treatment, the carrier used, and like factors within the knowledge and expertise of those skilled in the art. As appropriate, a therapeutically effective amount in any individual case can be determined by one of ordinary skill in the art by reference to the pertinent texts and literature and/or by using routine experimentation (see, e.g., Remington, The Science and Practice of Pharmacy (20^{th} ed. 2000)).

As a general proposition, a dosage from about 0.1 to about 5, 10, 20, 50, 75 or 100 mg/kg body weight will have therapeutic efficacy, with all weights being calculated based upon the weight of the active ingredient, including salts.

The pharmaceutical composition can be delivered in any suitable volume of administration. In representative embodiments of the invention, the administration volume for intranasal delivery ranges from about 25 microliters to 200 microliters or from about 50 to 150 microliters. Typically, the administration volume is selected to be small enough to allow for the dissolution of an effective amount of the active compound but sufficiently large to prevent therapeutically significant amounts of inhibitor from escaping from the anterior chamber of the nose and/or draining into the throat, post nasally.

Any suitable method of intranasal delivery can be employed for delivery of the pharmaceutical compound. In particular embodiments, intranasal administration is by inhalation (e.g., using an inhaler or nebulizer device), alternatively, by spray, tube, catheter, syringe, dropper, packtail, pledget, and the like. As a further illustration, the pharmaceutical composition can be administered intranasally as (1) nose drops, (2) powder or liquid sprays or aerosols, (3) liquids or semisolids by syringe, (4) liquids or semisolids by swab, pledget or other similar means of application, (5) a gel, cream or ointment, (6) an infusion, or (7) by injection, or by any means now known or later developed in the art. In particular embodiments, the method of delivery is by nasal drops, spray or aerosol.

In representative embodiments, the pharmaceutical formulation is directed upward during administration, to enhance delivery to the upper third (e.g., the olfactory epithelium in the olfactory region) and the side walls (e.g., nasal epithelium) of the nasal cavity. Further, orienting the subject's head in a tipped-back position or orienting the subject's body in Mygind's position or the praying-to-Mecca position can be used to facilitate delivery to the olfactory region.

Many devices are known in the art for nasal delivery. Exemplary devices-include bidirectional devices, particle dispersion devices, and chip-based ink-jet technologies. Optinose or Optimist (OptiNose, AS, Norway) and DirectHaler (Direct-Haler A/S, Denmark) are examples of bidirectional nasal delivery devices. ViaNase (Kurve Technolgies, Inc., USA) uses controlled particle dispersion technology. Ink-jet dispensers are described in U.S. Pat. No. 6,325,475 (MicroFab Technologies, Inc., USA) and use microdrops of drugs on a millimeter sized chip. Iontophoresis/phonophoresis/electrotransport devices are also known, as described in U.S. Pat. No. 6,410,046 (Intrabrain International NV, Curacao, AN). These devices comprise an electrode with an attached drug reservoir that is inserted into the nose. Iontophoresis, elctrotransport or phonophoresis with or without chemical permeation enhancers can be used to deliver the drug to the olfactory region.

The methods of intranasal delivery can be carried out once or multiple times, and can further be carried out daily, every other day, etc., with a single administration or multiple administrations per day of administration, (e.g., 2, 3, 4 or more times per day of administration). In other embodiments, the methods of the invention can be carried out on an as-needed by self-medication.

Further, the pharmaceutical compositions of the present invention can optionally be administered in conjunction with other therapeutic agents, for example, other therapeutic agents useful in the treatment of hyperglycemia, diabetes, metabolic syndrome and/or obesity. For example, the compounds of the invention can be administered in conjunction with insulin therapy and/or hypoglycemic agents (e.g., metformin). The additional therapeutic agent(s) can optionally be administered concurrently with the compounds of the invention, in the same or different formulations. As used herein, the word “concurrently” means sufficiently close in time to produce a combined effect (that is, concurrently can be simultaneously, or it can be two or more events occurring within a short time period before or after each other).

TABLE 1

| | Molecular | | | Mechanism of | |
|---|---|---|---|---|---|
| Chemical Name/Description | Formula | Generic Name | Brand Name | Action | Structure |
| 6-Chloro-3-(2-hydroxy-1,1- dimethylethylamino)-4H- thieno[3,2-e][1,2,4]thiadiazine-1,1-dioxide | C9H12ClN3O3S2 | | | K(ATP) Channel Activators | 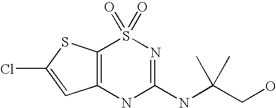 |
| 6-Chloro-3-(1,1,3,3- tetramethylbutylamino)-4H- thieno[3,2-e][1,2,4]thiadiazine- 1,1-dioxide | C13H20ClN3O2S2 | | | K(ATP) Channel Activators | 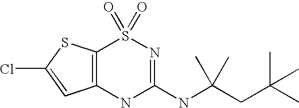 |
| 3-(1-Adamantylamino)-6-chloro- 4H-thieno[3,2-e][1,2,4]thiadiazine- 1,1-dioxide | C15H18ClN3O2S2 | | | K(ATP) Channel Activators | 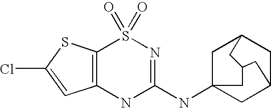 |
| 1-(6-Chloro-1,1-dioxo-4H- thieno[3,2-e][1,2,4]thiadiazin-3- ylamino)cyclopropanecarboxylic acid ethyl ester | C11H12ClN3O4S2 | | | K(ATP) Channel Activators | 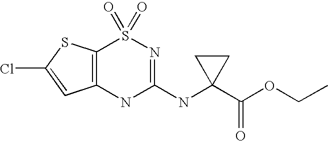 |
| 6-Chloro-3-(1-methyl-1- phenylethylamino)-4H-thieno[3,2- e][1,2,4]thiadiazine-1,1-dioxide | C14H14ClN3O2S2 | | | K(ATP) Channel Activators | 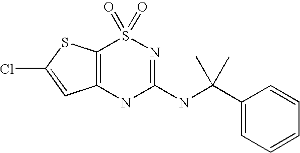 |
| 6-Chloro-3-[1- (hydroxymethyl)cyclopentylamino]- 4H-thieno[3,2-e][1,2,4]thiadiazine- 1,1-dioxide | C11H14ClN3O3S2 | | | K(ATP) Channel Activators | 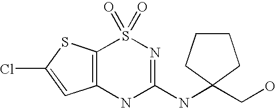 |
| 1-(6-Chloro-1,1-dioxo-4H- thieno[3,2-e][1,2,4]thiadiazin-3- ylamino)cyclopropanecarboxylic acid | C9H8ClN3O4S2 | | | K(ATP) Channel Activators | 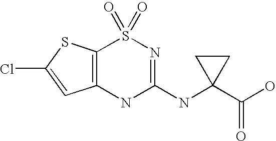 |
| 6-Chloro-3-(1- methylcyclobutylamino)-4H- thieno[3,2-e][1,2,4]thiadiazine-1,1- dioxyde | C10H12ClN3O2S2 | | | K(ATP) Channel Activators | 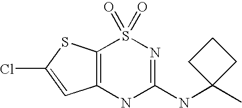 |
| 6-Chloro-3-(1- methylcyclohexylamino)-4H- thieno[3,2-e][1,2,4]thiadiazine-1,1- dioxyde | C12H16ClN3O2S2 | | | K(ATP) Channel Activators | 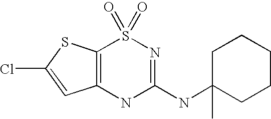 |
| 6-Chloro-3-(1- methylcyclopentylamino)-4H- thieno[3,2-e][1,2,4]thiadiazine-1,1- dioxyde | C11H14ClN3O2S2 | | | K(ATP) Channel Activators | 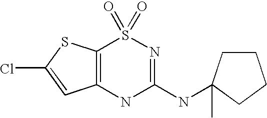 |
| 6-Chloro-3-(1- ethylcyclobutylamino)-4H- thieno[3,2-e][1,2,4]thiadiazine-1,1- dioxyde | C11H14ClN3O2S2 | | | K(ATP) Channel Activators | 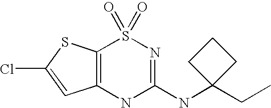 |
| 7-Chloro-3-methyl-4H-1,4- benzothiazine-2-carbonitrile 1,1- dioxide | C10H7ClN2O2S | | | K(ir) 6.2/SUR1 Activators | 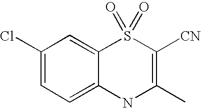 |
| 3-Methyl-6-(trifluoromethyl)-4H- 1,4-benzothiazine-2-carbonitrile 1,1-dioxide | C11H7F3N2O2S | | | K(ir) 6.2/SUR1 Activators | 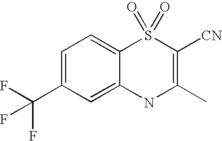 |
| 3-Methyl-6-(trifluoromethyl)-4H- 1,4-benzothiazine-2-carbonitrile 1- oxide | C11H7F3N2OS | | | K(ATP) Channel Activators | 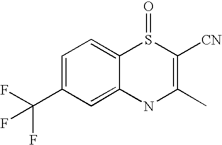 |
| 7-Methoxy-3-methyl-4H-1,4- benzothiazine-2-carbonitrile 1,1- dioxide | C11H10N3O3S | | | K(ATP) Channel Activators | 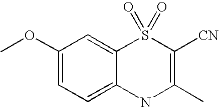 |
| 6-Fluoro-3-methyl-4H-1,4- benzothiazine-2-carbonitrile 1,1- dioxide | C10H7FN2O2S | | | K(ATP) Channel Activators | 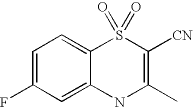 |
| 3-Propyl-6-(trifluoromethyl)-4H-1,4- benzothiazine-2-carbonitrile 1,1- dioxide | C13H11F3N2O2S | | | K(ATP) Channel Activators | 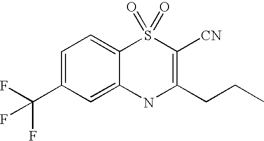 |
| 5,7-Difluoro-3-methyl-4H-1,4- benzothiazine-2-carbonitrile 1,1- dioxide | C10H6F2N2O2S | | | K(ATP) Channel Activators | 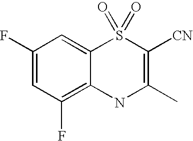 |
| 7-Fluoro-3-methyl-4H-1,4- benzothiazine-2-carbonitrile 1,1- dioxide | C10H7FN2O2S | | | K(ATP) Channel Activators | 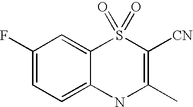 |
| 3-(2,4-Dimethoxyphenyl)-N-[2-[4- (2-methoxyethoxy)-3-(3- methylureidosulfonyl)phenyl]ethyl]- 2(E)-propenamide | C24H31N3O8S | | | K(ATP) Channel Blockers | 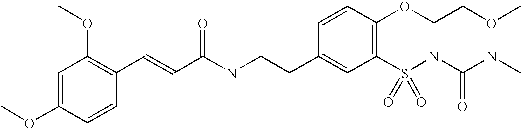 |
| 3-(2,4-Dimethoxyphenyl)-N-[2-[4- methoxy-3-(3- methylthioureidosulfonyl)phenyl]ethyl]-2(E)-propenamide | C22H27N3O6S2 | | | K(ATP) Channel Blockers | 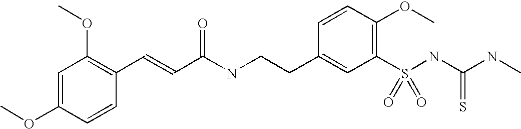 |
| 3-(2,4-Dimethylphenyl)-N-[2-[4- methoxy-3-(3- methylthioureidosulfonyl)phenyl]ethyl]-2(E)-propenamide | C22H27N3O4S2 | | | K(ATP) Channel Blockers | 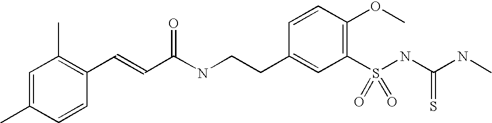 |
| 3-(2,4-Dimethoxyphenyl)-N-[2-[4- (2-methoxyethoxy)-3-(3- methylthioureidosulfonyl)phenyl]ethyl]-2(E)-propenamide | C24H31N3O7S2 | | | K(ATP) Channel Blockers | 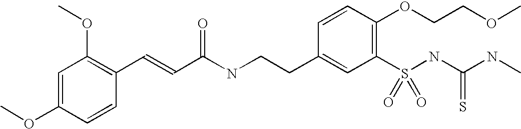 |
| 3-(2,4-Dimethoxyphenyl)-N-[2-[4- (2-methoxyethoxy)-3-(3- ethylthioureidosulfonyl) phenyl]ethyl]-2(E)-propenamide | C25H33N3O7S2 | | | K(ATP) Channel Blockers | 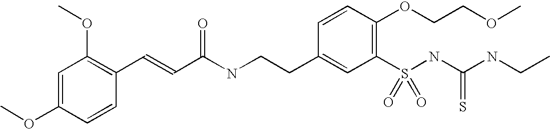 |
| N-[2-[4-Methoxy-3-(3- methylthioureidosulfonyl)phenyl]ethyl]-3-phenyl-2(E)-propenamide | C20H23N3O4S2 | | | K(ATP) Channel Blockers | 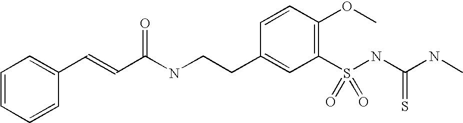 |
| 5-tert-Butyl-2-methoxy-N-[2-[4- methoxy-3-(3- methylthioureidosulfonyl)phenyl]ethyl]benzamide | C23H31N3O5S2 | | | K(ATP) Channel Blockers | 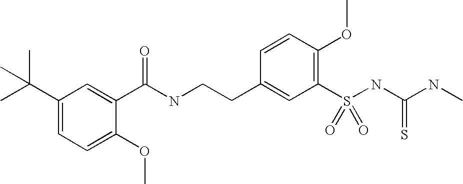 |
| (−)-9(S)-(3-Bromo-4-fluorophenyl)- 2,3,4,5,6,7,8,9- octahydrothieno[3,2-b]quinoline-8- one 1,1-dioxide | C17H15BrFNO3S | | | K(ATP) Channel Activators | 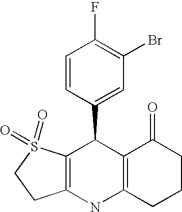 |
| 6-Chloro-N-isopropyl-4H- thieno[2,3-e][1,2,4]thiadiazin-3- amine 1,1-dioxide | C8H10ClN3O2S2 | | | K(ir) 6.2/SUR1 Activators | 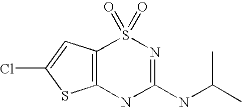 |
| N-[6-Amino-2-(dimethoxymethyl)- 3(S)-hydroxy-2(S)-methyl-3,4- dihydro-2H-1-benzopyran-4(R)-yl]- N′-benzyl-N″-cyanoguanidine | C22H27N5O4 | | | Antioxidants; K(ATP) Channel Activators | 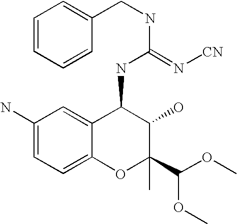 |
| N-[6-(Acetamido)-2- (dimethoxymethyl)-3(S)-hydroxy- 2(S)-methyl-3,4-dihydro-2H-1- benzopyran-4(R)-yl]-N′-benzyl-N″- cyanoguanidine | C24H29N5O5 | | | Antioxidants; K(ATP) Channel Activators | 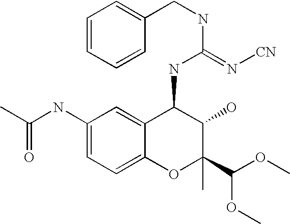 |
| 4-(3-Bromo-4-fluorophenyl)-1- ethyl-1,2,3,4,5,6,7,8- octahydrocyclopenta[b]pyrazolo[4,3- e]pyridine-3,5-dione | C17H15BrFN3O2 | | | K(ATP) Channel Activators | 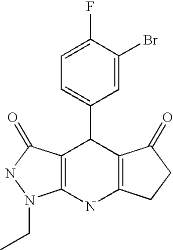 |
| 4-(4-Fluoro-3-iodophenyl)-1- methyl-1,2,3,4,5,6,7,8- octahydrocyclopenta[b]pyrazolo[4,3- e]pyridine-3,5-dione | C16H13FlN3O2 | | | K(ATP) Channel Activators | 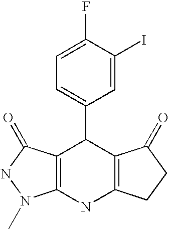 |
| 2,2-Dimethyl-4-(2-oxo-1,2- dihydropyridin-1-yl)-N-phenyl-2H- 1-benzopyran-6-sulfonamide | C22H20N2O4S | | | K(ATP) Channel Activators | 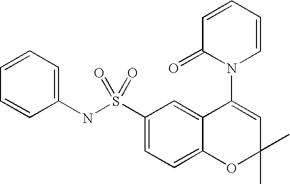 |
| N,2,2-Trimethyl-4-(2-oxo-1,2- dihydropyridin-1-yl)-N-phenyl-2H- 1-benzopyran-6-sulfonamide | C23H22N2O4S | | | K(ATP) Channel Activators | 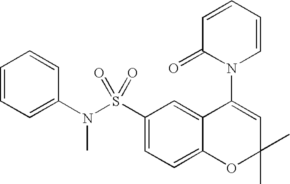 |
| 2,2-Dimethyl-4-(2-oxo-1,2- dihydropyridin-1-yl)-2H-1- benzopyran-6-sulfonic acid phenyl ester | C22H19NO5S | | | K(ATP) Channel Activators | 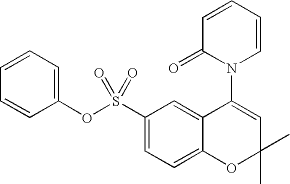 |
| N-[2-[4-(2-Methoxyethoxy)-3-(3- methylthioureidosulfonyl)phenyl]ethyl]quinoline-3-carboxamide | C23H26N4O5S2 | | | K(ATP) Channel Blockers | 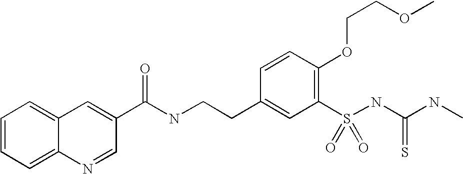 |
| N-[2-[3-(3-Methylthioureido- sulfonyl)-4-[2-(2,2,2- trifluoroethoxy)ethoxy]phenyl]ethyl]quinoline-3-carboxamide | C24H25F3N4O5S2 | | | K(ATP) Channel Blockers | 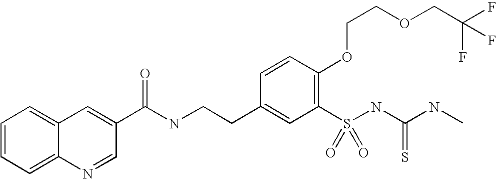 |
| N-[2-[3-(3-Methylthioureido- sulfonyl)-4-propoxyphenyl]ethyl]quinoline-3-carboxamide | C23H26N4O4S2 | | | K(ATP) Channel Blockers | 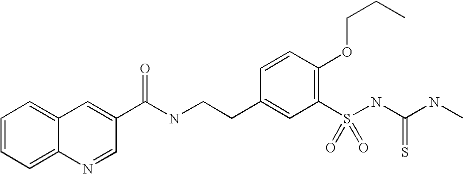 |
| N-[2-[4-(2-Methoxyethoxy)-3-(3- methylthioureidosulfonyl)phenyl]ethyl]-3-methyl-2-butenamide | C18H27N3O5S2 | | | K(ATP) Channel Blockers | 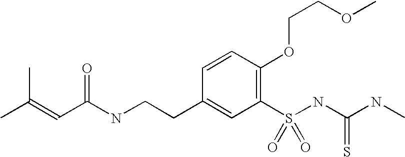 |
| N-[2-[4-(2-Methoxyethoxy)-3-(3- methylureidosulfonyl)phenyl]ethyl]quinoline-3-carboxamide | C23H26N4O6S | | | K(ATP) Channel Blockers | 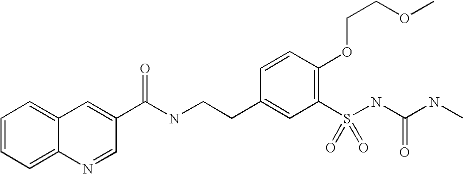 |
| N-[2-[3-(3-Methylthioureido- sulfonyl)-4-(2- phenoxyethoxy)phenyl]ethyl]quinoline-3-carboxamide | C28H28N4O5S2 | | | K(ATP) Channel Blockers | 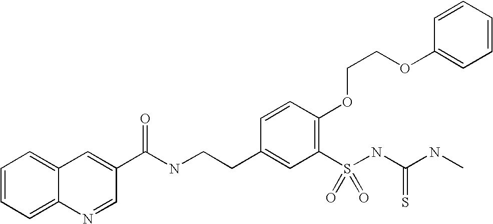 |
| Isopropylthioureido- sulfonyl)-4-methoxyphenyl]ethyl]-1- cyclohexene-1-carboxamide | C20H29N3O4S2 | | | K(ATP) Channel Blockers | 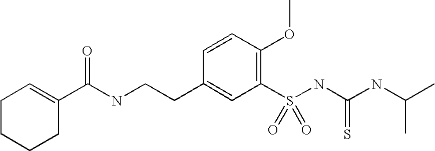 |
| N-[2-[3-(3-Cyclohexylthioureido- sulfonyl)-4-methoxyphenyl]ethyl]-1- cyclohexene-1-carboxamide | C23H33N3O4S2 | | | K(ATP) Channel Blockers | 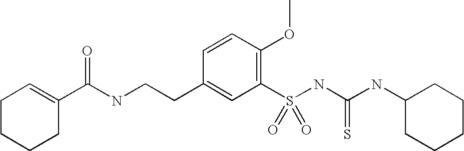 |
| N-[2-[4-Methoxy-3-(3- methylureidosulfonyl)phenyl]ethyl]- 3-(2-thienyl)-2(E)-propenamide | C18H21N3O5S2 | | | K(ATP) Channel Blockers | 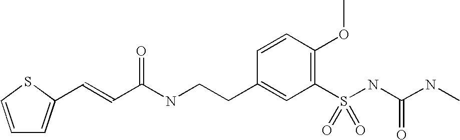 |
| N-[2-[4-(2-Methoxyethoxy)-3-(3- methylureidosulfonyl)phenyl]ethyl]- 3-(2-thienyl)-2(E)-propenamide | C20H25N3O6S2 | | | K(ATP) Channel Blockers | 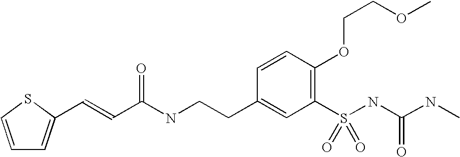 |
| N-[2-[4-(2-Ethoxyethoxy)-3-(3- methylthioureidosulfonyl)phenyl]ethyl]-3-(2-thienyl)-2(E)- propenamide | C21H27N3O5S3 | | | K(ATP) Channel Blockers | 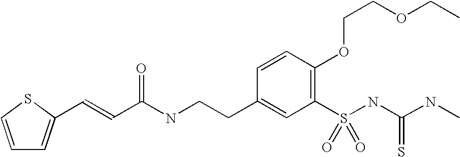 |
| N-[2-[3-(3-Isopropylthioureido- sulfonyl)-4-(2-methoxyethoxy) phenyl]ethyl]-3-(2-pyridyl)- 2(E)-propenamide | C23H30N4O5S2 | | | K(ATP) Channel Blockers | 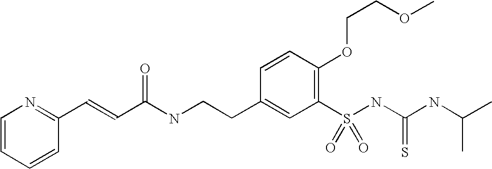 |
| 3-(Isopropylamine)-7-methoxy-4H- 1,2,4-benzothiadiazine 1,1-dioxide | C11H15N3O3S | | | Insulin Lowering Agents; K(ir) 6.2/SUR1 Activators | 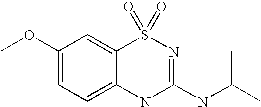 |
| 3,5-Dichloro-N-[1(S)-[2-(2- chloropyridin-3-ylamino)-3,4-dioxo- 1-cyclobuten-1-ylamino]-2,2- dimethylpropyl]benzamide | C21H19Cl3N4O3 | | | K(ATP) Channel Activators | 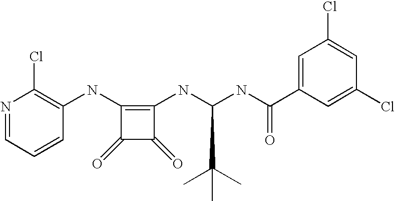 |
| N-[2-[2,2,2-Trifluoro-1-hydroxy-1- (trifluoromethyl)ethyl]naphthalen-1- yl]acetamide | C15H11F6NO2 | | | K(ATP) Channel Activators | 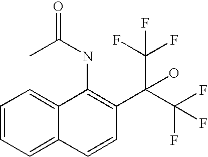 |
| 5-Chloro-2-methoxy-N-[2-[2-(3- methylthioureidosulfonyl)biphenyl- 4-yl]ethyl]benzamide | C24H24ClN3O4S2 | | | K(ATP) Channel Blockers | 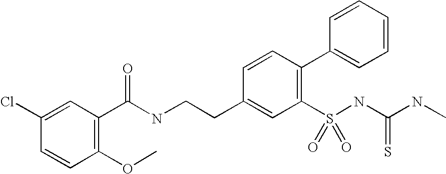 |
| 5-Chloro-2-methoxy-N-[2-[2-(3- methylureidosulfonyl)biphenyl-4- yl]ethyl]benzamide | C24H24ClN3O5S | | | K(ATP) Channel Blockers | 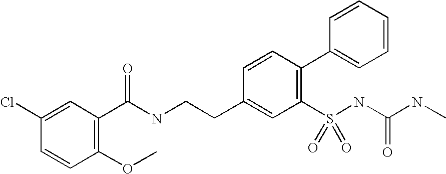 |
| 5-Chloro-N-[2-[4′-fluoro-2-(3- methylureidosulfonyl)biphenyl-4- yl]ethyl]-2-methoxybenzamide | C24H23ClFN3O5S | | | K(ATP) Channel Blockers | 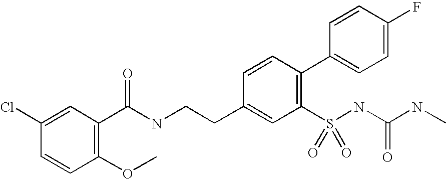 |
| 5-Chloro-2-methoxy-N-[2-[3-(3- methylureidosulfonyl)-4-(2- thienyl)phenyl]ethyl]benzamide | C22H22ClN3O5S2 | | | K(ATP) Channel Blockers | 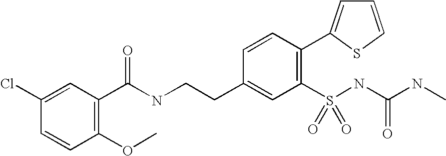 |
| 5-Chloro-N-[2-[4-(2-furyl)-3-(3- methylureidosulfonyl)phenyl]ethyl]- 2-methoxybenzamide | C22H22ClN3O6S | | | K(ATP) Channel Blockers | 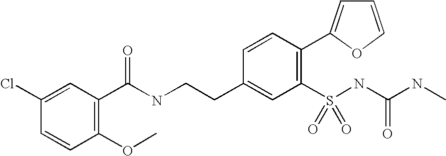 |
| 5-Chloro-2-methoxy-N-[2-[3-(3- methylthioureidosulfonyl)-4-(2- pyridyl)phenyl]ethyl]benzamide | C23H23ClN4O4S2 | | | K(ATP) Channel Blockers | 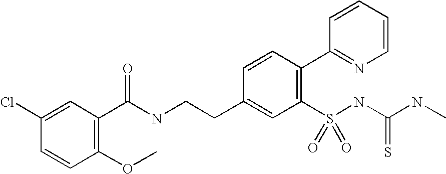 |
| 5-Chloro-2-methoxy-N-[2-[3-(3- methylthioureidosulfonyl)-4- (phenylsulfanyl)phenyl]ethyl]benzamide | C24H24ClN3O4S3 | | | K(ATP) Channel Blockers | 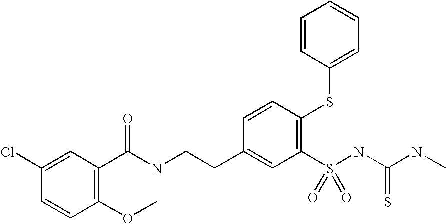 |
| 5-[(E)-(2,4-Dioxothiazolidin-5- ylidenemethyl)-N-[1(S)- phenylethyl]-2-propoxybenzamide | C22H22N2O4S | | | K(ATP) Channel Activators | 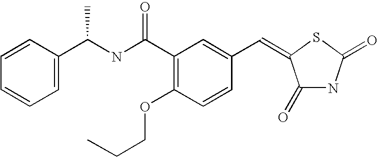 |
| 7-Chloro-3-(1-phenylethylsulfanyl)- 4H-1,2,4-benzothiadiazine 1,1- dioxide | C15H13ClN2O2S2 | | | K(ATP) Channel Activators | 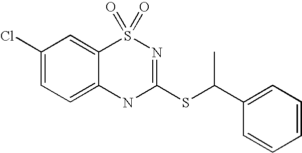 |
| 2-(2,2-Dimethyl-6-nitro-3,4- dihydro-2H-1,4-benzothiazin-4-yl)- 2-cyclopenten-1-one | C15H16N2O3S | | | K(ATP) Channel Activators | 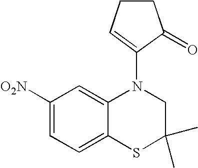 |
| 2,2-Dimethyl-4-(5-oxo-1- cyclopenten-1-yl)-3,4-dihydro-2H- 1,4-benzothiazine-6-carbonitrile | C16H16N2OS | | | K(ATP) Channel Activators | 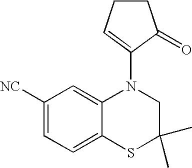 |
| 2-[2,2-Dimethyl-6-(trifluoromethyl)- 3,4-dihydro-2H-1,4-benzothiazin-4- yl]-2-cyclopenten-1-one | C16H16F3NOS | | | K(ATP) Channel Activators | 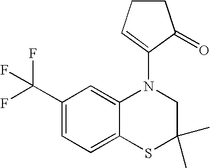 |
| 4-Chloro-N-[2,2-dichloro-1-[3-(6- chloropyridin-3-yl)-2- cyanoguanidino]propyl]benzamide | C17H14Cl4N6O | | | K(ATP) Channel Activators | 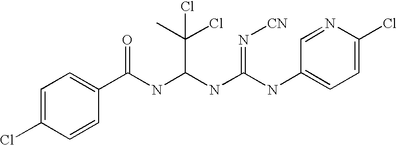 |
| (+)-4-(3-Bromo-4-fluorophenyl)-1- methyl-3,4,5,6,8,9-hexahydro-1H- isoxazolo[3,4-b]pyrano[4,3- e]pyridine-3,5-dione | C16H12BrFN2O4 | | | K(ATP) Channel Activators | 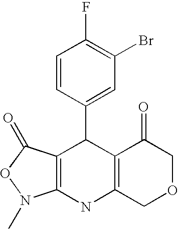 |
| 6,7-Dichloro-N-isopropyl-4H-1,2,4- benzothiadiazin-3-amine 1,1- dioxide | C10H11Cl2N3O2S | | | Insulin Lowering Agents; K(ATP) Channel Activators | 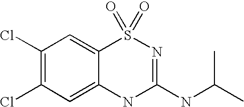 |
| N-(2,6-Dichlorophenyl)-N-(4- fluorobenzyl)-4,5-dihydro-1H- imidazol-2-amine | C16H14Cl2FN3 | | | K(ATP) Channel Blockers | 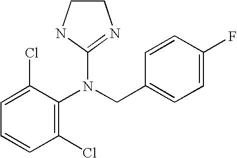 |
| 9-(3,4-Dichlorophenyl)- 1,2,3,4,5,6,7,8,9,10- decahydroacridine-1,8-dione | C19H17Cl2NO2 | | | K(ATP) Channel Activators | 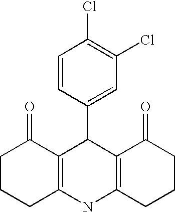 |
| 3-[3,5-Bis(trifluoromethyl) phenylamino]-2-(4- chlorophenylsulfonyl)-3- [1(R),2,2-trimethylpropylamino]- 2(E)-propenenitrile | C23H22ClF6N3O2S | | | K(ATP) Channel Activators | 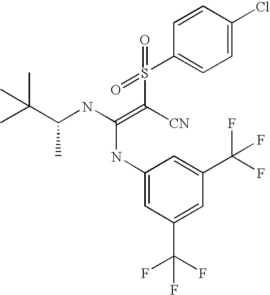 |
| N-[2-[2,2,2-Trifluoro-1-hydroxy-1- (trifluoromethyl)ethyl]naphthalen-1- yl]furan-2-carboxamide | C18H11F6NO3 | | | K(ATP) Channel Activators | 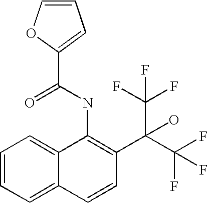 |
| N-Isopropyl-N-(1,1,2- trimethylpropyl)amine hydrochloride; 2,3-Dimethyl-N- isopropyl-2-butanamine hydrochloride | C9H22ClN | Iptakalim hydrochloride | | K(ATP) Channel Activators | 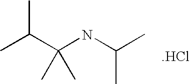 |
| N-Ethyl-7-fluoro-4H-1,2,4- benzothiadiazin-3-amine 1,1- dioxide | C9H10FN3O2S | | | K(ATP) Channel Activators | 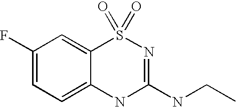 |
| 7-Chloro-N-(1,1-dimethylpropyl)- 4H-1,2,4-benzothiadiazin-3-amine 1,1-dioxide | C12H16ClN3O2S | | | K(ATP) Channel Activators | 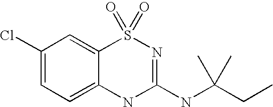 |
| N-Cyclopentyl-7-fluoro-4H-1,2,4- benzothiadiazin-3-amine 1,1- dioxide | C12H14FN3O2S | | | K(ATP) Channel Activators | 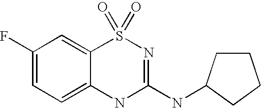 |
| (−)-4-Chloro-N-[2,2-dichloro-1(R)- [1-(cyanoimino)-1-(pyridin-3- ylamino)methylamino]propyl]benzamide | C17H15Cl3N6O | | | K(ATP) Channel Activators | 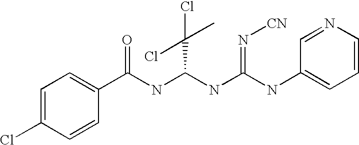 |
| 3-(2,4-Dinitrophenyl)-4-nitro-5- (trifluoromethyl)-1H-pyrazole | C10H4F3N5O6 | | | K(ir) 6.2/SUR1 Activators | 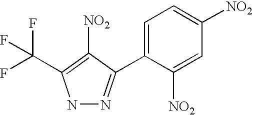 |
| 1-[3,5-Bis(trifluoromethyl)phenyl]- 2-cyano-3-cyclopentylguanidine | C15H14F6N4 | | | K(ATP) Channel Blockers; K(ir) 6.2/SUR1 Activators | 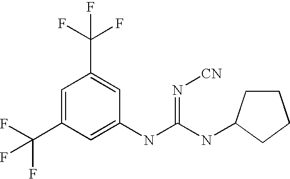 |
| 4-(3-Bromo-4-fluorophenyl)-1- methyl-1,3,4,5,7,8- hexahydrofuro[3,4-b]isoxazolo[4,3- e]pyridine-3,5-dione | C15H10BrFN2O4 | | | K(ATP) Channel Activators | 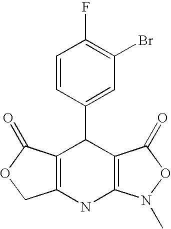 |
| 9(S)-(3-Bromo-4-fluorophenyl)- 1,3,4,5,6,7,8,9-octahydrofuro[3,4- b]quinoline-1,8-dione | C17H13BrFNO3 | | | K(ATP) Channel Activators | 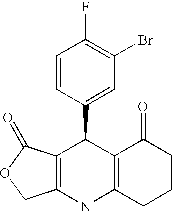 |
| (−)-9-(3-Bromo-4-fluorophenyl)- 3,4,5,6,8,9-hexahydro-2H-furo[3,4- b]thiino[2,3-e]pyridin-8-one 1,1- oxide | C16H13BrFNO4S | | | K(ATP) Channel Activators | 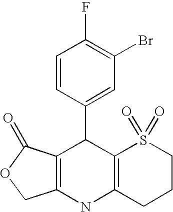 |
| 9(S)-(3-Bromo-4-fluorophenyl)- 3,4,5,7,8,9-hexahydro-1H-furo[3,4- b]pyrano[4,3-e]pyridine-1,8-dione | C16H11BrFNO4 | | | K(ATP) Channel Activators | 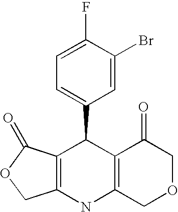 |
| 7-Chloro-N-isopropyl-4H-1,2,4- benzothiadiazin-3-amine 1,1- dioxide | C10H12ClN3O2S | | | K(ATP) Channel Activators | 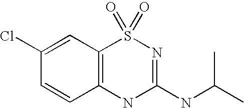 |
| 6-Chloro-N-cyclobutyl-7-fluoro-4H- 1,2,4-benzothiadiazin-3-amine 1,1- dioxide | C11H11ClFN3O2S | | | K(ATP) Channel Activators | 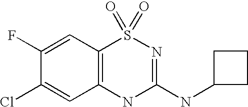 |
| N-(6-Chloro-2,2-dimethyl-3,4- dihydro-2H-1-benzopyran-4- yl)acetamide | C13H16ClNO2 | | | K(ATP) Channel Activators | 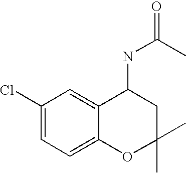 |
| Spartein-2-one; (7S,7aR,14S,14aS)-7,14- Methanoperhydrodipyrido[1,2- a:1′,2′-e][1,5]diazocin-11-one | C15H24N2O | Lupanin; (+)-2- Oxosparteine; Lupanine | | Alkaloids; Insulin Secretagogues; K(ATP) Channel Blockers; Sodium Channel Blockers | 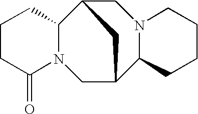 |
| (2S,7S,7aR,14S,14aS)-2-Hydroxy- 7,14-methanoperhydro- dipyrido[1,2- a:1′,2′-e][1,5]diazocin-11-one | C15H24N2O2 | 13alpha- Hydroxylupanine; 13-Hydroxy- lupanine; Oxylupanine; 13- Hydroxylupanin | | Alkaloids; Insulin Secretagogues; K(ATP) Channel Blockers | 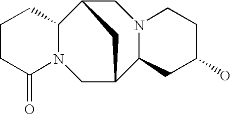 |
| Sparteine-2,17-dione; (7S,7aS,14R,14aR)-7,14- Methanoperhydrodipyrido[1,2- a: 1′,2′-e][1,5]diazocine-4,13-dione | C15H22N2O2 | 17-Oxolupanine; (+)-17- Oxolupanine; (+)-2,17- Dioxosparteine | | Alkaloids; Insulin Secretagogues; K(ATP) Channel Blockers | 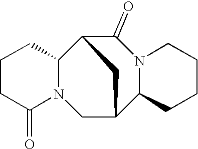 |
| Sparteine-2-thione; (7S,7aR,14S,14aS)-7,14- Methanoperhydrodipyrido[1,2- a: 1′,2′-e][1,5]diazocine-1,1-thione | C15H24N2S | (+)-2- Thionosparteine | | Alkaloids; Insulin Secretagogues; K(ATP) Channel Blockers; Metal Chelators | 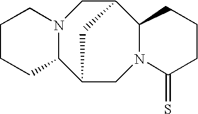 |
| 3,3,3-Trifluoro-2-hydroxy-2-methyl- N-[4-(phenylsulfonyl)phenyl]propionamide | C16H14F3NO4S | | | K(ATP) Channel Activators | 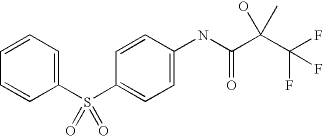 |
| N-Isopropyl-7-pentyl-4H-1,2,4- benzothiadiazin-3-amine 1,1- dioxide | C15H23N3O2S | | | K(ATP) Channel Activators | 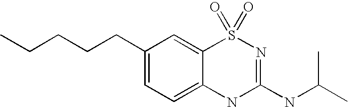 |
| N-Propyl-7-(trifluoromethyl)-4H- 1,2,4-benzothiadiazin-3-amine 1,1- dioxide | C11H12F3N3O2S | | | K(ATP) Channel Activators | 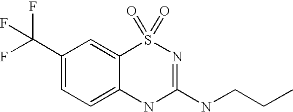 |
| N-Isopropyl-7-(trifluoromethyl)-4H- 1,2,4-benzothiadiazin-3-amine 1,1- dioxide | C11H12F3N3O2S | | | Insulin Lowering Agents; K(ATP) Channel Activators | 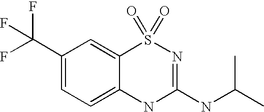 |
| N-(1-Methylpropyl)-7- (trifluoromethyl)-4H-1,2,4- benzothiadiazin-3-amine 1,1- dioxide | C12H14F3N3O2S | | | K(ATP) Channel Activators | 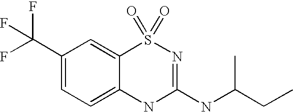 |
| 6-Chloro-7-fluoro-N-isopropyl-4H- 1,2,4-benzothiadiazin-3-amine 1,1- dioxide | C10H11ClFN3O2S | | | K(ATP) Channel Activators | 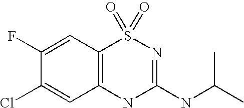 |
| 9(R)-(3-Iodo-4-methylphenyl)- 4,5,7,9-tetrahydro-3H-furo[3,4- b]pyrano[4,3-e]pyridine-1,8-dione | C17H14lNO4 | | | K(ATP) Channel Activators | 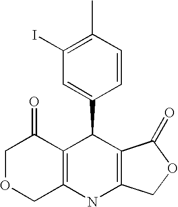 |
| N-(N-Butylcarbamoyl)-4- methylbenzenesulfonamide; 1- Butyl-3-(4- methylphenylsulfonyl)urea | C12H18N2O3S | Tolbutamide | Orinase; Dolipol | K(ATP) Channel Blockers; Sulfonylureas | 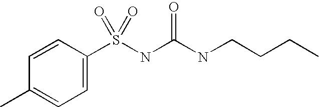 |
| (±)-trans-6-Cyano-2,2-dimethyl-4- (2-oxo-1-pyrrolidinyl)-3,4-dihydro- 2H-benzo[b]pyran-3-ol | C16H18N2O3 | Cromakalim | | K(ATP) Channel Activators | 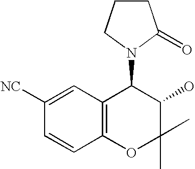 |
| (±)-N′-Cyano-N-4-pyridinyl-N″- (1,2,2-trimethylpropyl)guanidine monohydrate | C13H21N5O | Pinacidil | Pindac | K(ATP) Channel Activators | 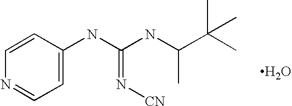 |
| 1-(4-Chlorophenylsulfonyl)-3- propylurea; 4-Chloro-N-(N- propylcarbamoyl)benzenesulfon- amide | C10H13ClN3O3S | Chlorpropamide | Diabinese | K(ATP) Channel Blockers; Sulfonylureas | 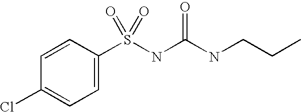 |
| 1-(4-Acetylphenylsulfonyl)-3- cyclohexylurea; 4-Acetyl-N-(N- cyclohexylcarbamoyl)benzene- sulfonamide | C15H20N2O4S | Acetohexamide | Dymelor; Dimelor | K(ATP) Channel Blockers; Sulfonylureas | 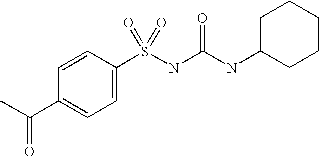 |
| 4-Methyl-N-[N-(perhydroazepin-1- yl)carbamoyl]benzenesulfon- amide; 1-(4-Methylphenylsulfonyl)- 3-(perhydroazepin-1-yl)urea | C14H21N3O3S | Tolazamide | Tolinase | K(ATP) Channel Blockers; Sulfonylureas | 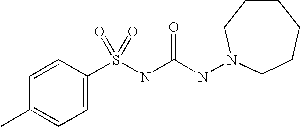 |
| 5-Chloro-N-[2-[4-(3- cyclohexylureidosulfonyl)phenyl]ethyl]-2-methoxybenzamide | C23H28ClN3O5S | Glibenclamide; Glyburide | Euglucan; Daonil; Euglucon; Diabeta; Micronase | K(ATP) Channel Blockers; Sulfonylureas | 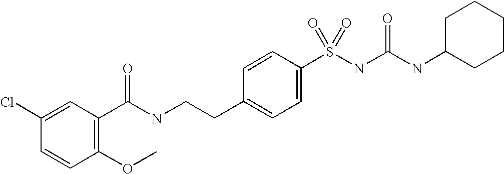 |
| N-[2-[4-(3-Cyclohexylureido- sulfonyl)phenyl]ethyl]-5- methylpyrazine-2-carboxamide | C21H27N5O4S | Glipizide | Minidiab; Minodiab; Glucotrol XL; Glucotrol | K(ATP) Channel Blockers; Sulfonylureas | 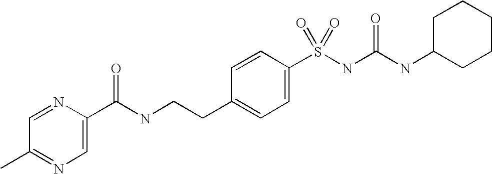 |
| 4-Methyl-N-[N- (perhydrocyclopenta[c]pyrrol-2- yl)carbamoyl]benzenesulfon-amide | C15H21N3O3S | Gliclazide | Glimicron HA; Glimicron; Gluctam; Diamicron; Glyzide | K(ATP) Channel Blockers; Sulfonylureas | 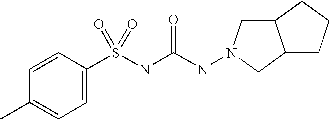 |
| N-Cyclohexyl-N′-[4-[2-(7-methoxy- 4,4-dimethyl-1,3-dioxo-1,2,3,4- tetrahydroisoquinolin-2- yl)ethyl]phenylsulfonyl]urea | C27H33N3O6S | Gliquidone | Glurenorm; Beglynorm | K(ATP) Channel Blockers; Sulfonylureas | 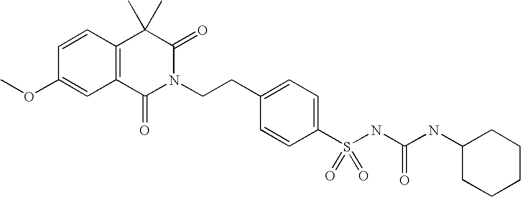 |
| 1-[4-[2-(3-Ethyl-4-methyl-2-oxo- 2,5-dihydro-1H-pyrrol-1- carboxamido)ethyl]phenylsulfonyl]- 3-(trans-4-methylcyclohexyl)urea | C24H34N4O5S | Glimepiride | Amaryl; Roname; Amarel; Glymepirid | K(ATP) Channel Blockers; Sulfonylureas | 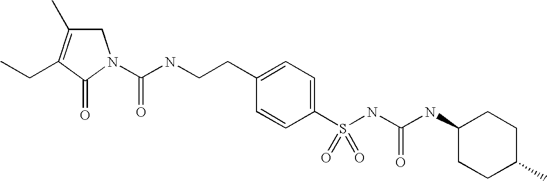 |
| 6-Amino-1,2-dihydro-1-hydroxy-2- imino-4-piperidinopyrimidine | C9H15N5O | Minoxidil | Regaine; Rogaine; Loniten; Neoxidil; Minoximen; RiUP | K(ATP) Channel Activators | 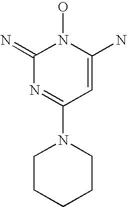 |
| (−)-N-(trans-4-Isopropylcyclohexyl- 1-carbonyl)-D-phenylalanine | C19H27NO3 | Nateglinide | Fastic; Starsis; Starlix; Trazec | Insulin Secretagogues; K(ir) 6.2/SUR1 Blockers | 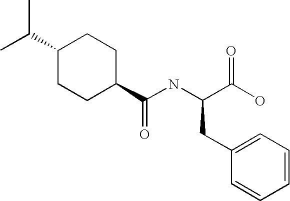 |
| (1S,5S,6R,7R)-5-[7-Hydroxy-6- [3(S)-hydroxy-3-methyl-1(E)- octenyl]bicyclo[3.3.0]oct-2-en-3- yl]pentanoic acid; 15-Deoxy-16(S)- hydroxy-16-methylisocarbacyclin; 15-Deoxy-16(S)-hydroxy-16- methyl-9a-O-methano-DELTA6,9a- prostaglandin I1 | C22H36O4 | | | K(ATP) Channel Activators; Prostanoid EP3 Agonists | 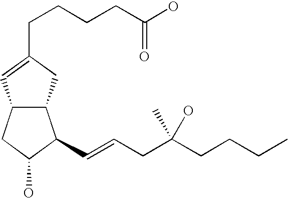 |
| N-(Cyclohexylcarbamoyl)-4-[2-[[(1- methyl-2-oxoquinolin-3- yl)carbonyl]amino]ethyl]benzene- sulfonamide; N-Cyclohexyl-N′-[4-[2- [[(1-methyl-2-oxoquinolin-3- yl)carbonyl]amino]ethyl]phenyl- sulfonyl]urea | C26H30N4O5S | | | K(ATP) Channel Blockers; Sulfonylureas | 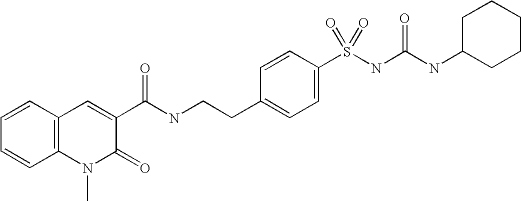 |
| N-(Cyclopentylcarbamoyl)-4-[2-(1- methyl-2-oxoquinolin-3- ylcarboxamido)ethyl]benzene- sulfonamide | C25H28N4O5S | | | K(ATP) Channel Blockers; Sulfonylureas | 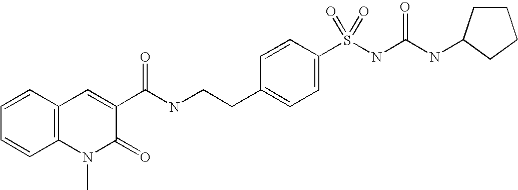 |
| N-(3-Methylcyclopentylcarbamoyl)- 4-[2-(1-methyl-2-oxoquinolin-3- ylcarboxamido)ethyl]benzene- sulfonamide | C26H30N4O5S | | | K(ATP) Channel Blockers; Sulfonylureas | 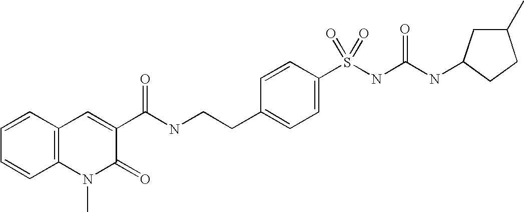 |
| N-(4-Methylcyclohexylcarbamoyl)- 4-[2-(1-methyl-2-oxoquinolin-3- ylcarboxamido)ethyl]benzene- sulfonamide | C27H32N4O5S | | | K(ATP) Channel Blockers; Sulfonylureas | 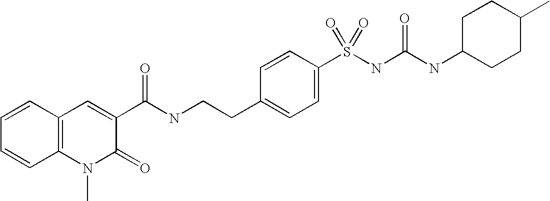 |
| N-(3-Cyclohexenylcarbamoyl)-4-[2- (1-methyl-2-oxoquinolin-3- ylcarboxamido)ethyl]benzene- sulfonamide | C26H28N4O5S | | | K(ATP) Channel Blockers; Sulfonylureas | 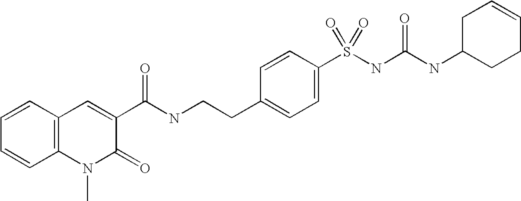 |
| N-(beta-Hydroxyethyl)nicotinamide nitrate ester; N-(2- Hydroxyethyl)pyridine-3- carboxamide nitrate ester | C8H9N3O4 | Nicorandil | Sigmart; Dancor; Ikorel; Adancor | K(ATP) Channel Activators | 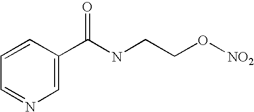 |
| 2,2-Dimethyl-4-(2-oxo-1,2- dihydropyridin-1-yl)-2H-1- benzopyran-6-carbonitrile; 2,2- Dimethyl-4-(2-oxo-1,2- dihydropyridin-1-yl)-2H-chromene- 6-carbonitrile | C17H14N2O2 | Bimakalim | | K(ATP) Channel Activators | 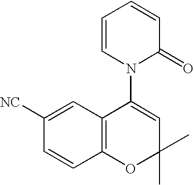 |
| (−)-trans-(3S,4R)-2-[3-Hydroxy-2,2- dimethyl-6-(trifluoromethoxy)-3,4- dihydro-2H-1-benzopyran-4-yl]- 2,3-dihydro-1H-isoindol-1-one | C20H18F3NO4 | Celikalim | | K(ATP) Channel Activators | 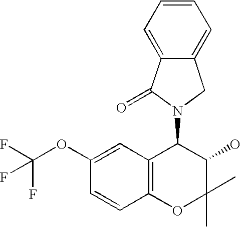 |
| (+)-7alpha-Hydroxy-6,6-dimethyl- 8beta-(2-oxopiperidin-1-yl)-7,8- dihydro-6H-pyrano[2,3-f]benzo- 2,1,3-oxadiazole | C16H19N3O4 | | | K(ATP) Channel Activators | 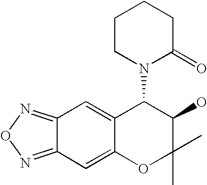 |
| (−)-(1R,2R)-N-Methyl-2-(3- pyridinyl)tetrahydro-2H-thiopyran- 2-carbothioamide 1-oxide | C12H16N2OS2 | Aprikalim | Aprim | K(ATP) Channel Activators | 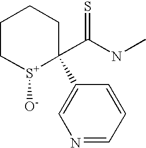 |
| (3S,4R)-5-Cyano-2,2-dimethyl-4- (2-oxo-1,2-dihydro-1-pyridyl)-3,4- dihydro-2H-benzo[b]pyran-3-ol; (3S,4R)-5-Cyano-2,2-dimethyl-4- (2-oxo-1,2-dihydro-1-pyridyl) chroman-3-ol; | | | | | |
| # (3S,4R)-3-Hydroxy- 2,2-dimethyl-4-(2-oxo-1,2-dihydro- pyridin-1-yl)-3,4-dihydro-2H-1- benzopyran-6-carbonitrile; (−)- (3S,4R)-3-Hydroxy-2,2-dimethyl- 4-(2-oxo-1,2-dihydropyridin-1- yl)chroman-6-carbonitrile | C17H16N2O3 | Emakalim | | K(ATP) Channel Activators | 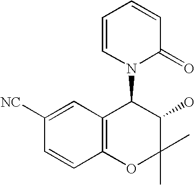 |
| (−)-(3S,4R)-3-Hydroxy-4-(1-methyl- 6-oxo-1,6-dihydropyridazin-3-yloxy)- 2,2-dimethyl-2H-3,4-dihydrobenzo[b]pyran-6-carbonitrile; (−)-(3S,4R)-3- Hydroxy-2,2-dimethyl-4-(1-methyl-6- oxo-1,6-dihydro-3-pyridazinyloxy) chroman-6-carbonitrile; | | | | | |
| # (−)-(3S,4R)- 3-Hydroxy-2,2-dimethyl-4-(1-methyl- 6-oxo-1,6-dihydropyridazin-3-yloxy)- 3,4-dihydro-2H-1-benzopyran-6- carbonitrile | C17H17N3O4 | | | K(ATP) Channel Activators | 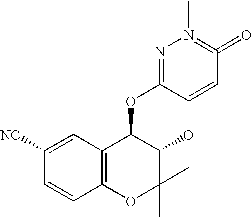 |
| 2-(2,2-Dimethyl-6-nitro-3,4- dihydro-2H-1,4-benzoxazin-4- yl)pyridine 1-oxide | C15H15N3O4 | | | K(ATP) Channel Activators | 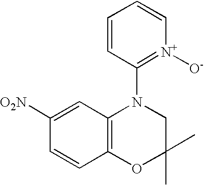 |
| (+)-1-[(3S,4R)-3-Hydroxy-2,2- dimethyl-6-(phenylsulfonyl)-4- chromanyl]pyrrolidin-2-one hemihydrate; (+)-(3S,4R)-3- Hydroxy-2,2-dimethyl-4-(2-oxo-1- pyrrolidinyl)-6-(phenylsulfonyl) chroman hemihydrate | C21H25NO6S | Rilmakalim hemihydrate | | 4K(ATP) Channel Activators | 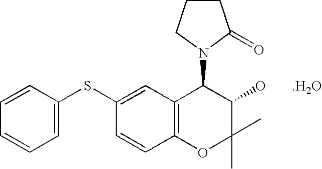 |
| N1-(1-Adamantyl)-N2- cyclohexylmorpholinocarbox- amidine hydrochloride | C21H36ClN3O | | | K(ATP) Channel Blockers | 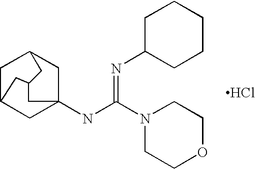 |
| 1-Cyano-2-(1,1-dimethylpropyl)-3- (3-pyridyl)guanidine | C12H17N5 | | | K(ATP) Channel Activators | 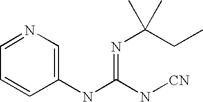 |
| (R)-(−)-2-[4-(4-Methyl-6-oxo- 1,4,5,6-tetrahydropyridazin-3- yl)phenylhydrazono]propanedi- nitrile; | | | | | |
| # (R)-(−)-2-[4-(4-Methyl-6- oxo-1,4,5,6-tetrahydropyridazin-3- yl)phenylhydrazono]malonodi- nitrile; (R)-(−)-6-[4-(1,1- Dicyanomethylenehydrazino) phenyl]-5-methyl-2,3,4,5- tetrahydropyridazin-3-one | C14H12N6O | Levosimendan | Simdax | Calcium Sensitizers; K(ATP) Channel Activators | 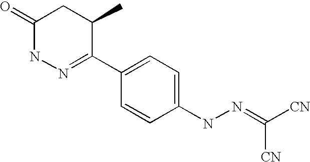 |
| (3S-trans)-N1-(4-Chlorophenyl)- N2-cyano-N3-(6-cyano-3-hydroxy- 2,2-dimethyl-3,4-dihydro-2H-1- benzopyran-4-yl)guanidine | C20H18ClN5O2 | | | K(ATP) Channel Activators | 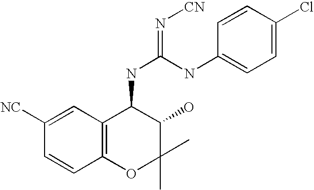 |
| trans-N2-Cyano-N1-(6-cyano-3- hydroxy-2,2-dimethyl-3,4-dihydro- 2H-1-benzopyran-4-yl)-N3-(1,1- dimethylpropyl)guanidine | C19H25N5O2 | | | K(ATP) Channel Activators | 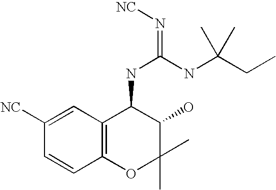 |
| 1-[3-(N-Hydroxyacetamidomethyl)- 2,2-dimethyl-6-(trifluoromethyl)-2H- 1-benzopyran-4-yl]pyridin-2(1H)- one; N-[2,2-Dimethyl-4-(2-oxo-1,2- dihydro-1-pyridyl)-6-trifluoromethyl- 2H-1-benzopyran-3- ylmethyl]acetohydroxamic acid | C20H19F3N2O4 | Sarakalim | | K(ATP) Channel Activators; Lipoxygenase Inhibitors | 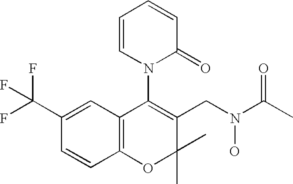 |
| 6,6-Dimethyl-8-(N-oxido-2-pyridyl)- 7,8-dihydro-6H-furazan[3,4- g]benz[1,4]oxazine | C15H14N4O3 | | | K(ATP) Channel Activators | 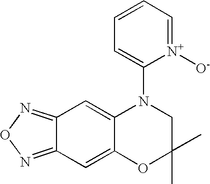 |
| (−)-(S)-N-(4-Benzoylphenyl)-3,3,3- trifluoro-2-hydroxy-2- methylpropionamide | C17H14F3NO3 | | | K(ATP) Channel Activators | 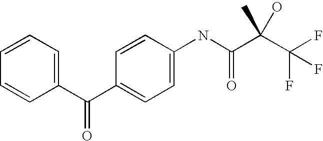 |
| 3-(1,8-Dioxo-1,2,3,4,5,6,7,8,9,10- decahydroacridin-9-yl)benzonitrile; 9-(3-Cyanophenyl)- 1,2,3,4,5,6,7,8,9,10- decahydroacridine-1,8-dione | C20H18N2O2 | | | K(ATP) Channel Activators | 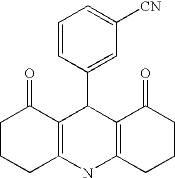 |
| N2-Cyano-N1-[1(R)-phenylpropyl]- N3-(3-pyridyl)guanidine | C16H17N5 | | | K(ATP) Channel Blockers | 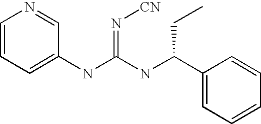 |
| N2-Cyano-N1-(1-phenylcyclobutyl)- N3-(3-pyridyl)guanidine | C17H17N5 | | | K(ATP) Channel Blockers | 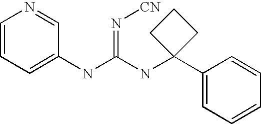 |
| N1-[1-(3-Chlorophenyl)propyl]-N2- cyano-N3-(3-pyridyl)guanidine | C16H16ClN5 | | | K(ATP) Channel Blockers | 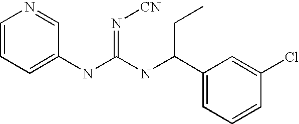 |
| N2-Cyano-N1-(1-phenylcyclopropyl)- N3-(3-pyridyl)guanidine | C16H15N5 | | | K(ATP) Channel Blockers | 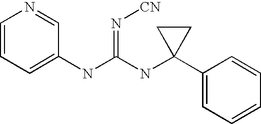 |
| (−)-3-[5-Oxo-2-(trifluoromethyl)- 1,4,5,6,7,8-hexahydroquinolin- 4(S)-yl]benzonitrile | C17H13F3N2O | | | K(ATP) Channel Activators | 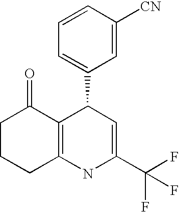 |
| 3-(1,2-Dimethylpropylamino)-4H- pyrido[4,3-e]-1,2,4-thiadiazine-1,1- dioxide | C11H16N4O2S | | | K(ATP) Channel Activators | 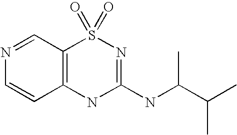 |
| trans-4-[N-(4-Chlorophenyl)-N-(1H- imidazol-2-ylmethyl)amino]-3(R)- hydroxy-2,2-dimethyl-3,4-dihydro- 2H-1-benzopyran-6-carbonitrile hydrochloride | C22H22Cl2N4O2 | | | K(ATP) Channel Activators | 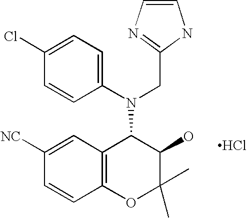 |
| 5-Chloro-2-methoxy-N-[2-[4-(2- methoxyethoxy)-3-(3- methylthioureidosulfonyl)phenyl]ethyl]benzamide | C21H25ClN3NaO6S2 | | | K(ATP) Channel Blockers | 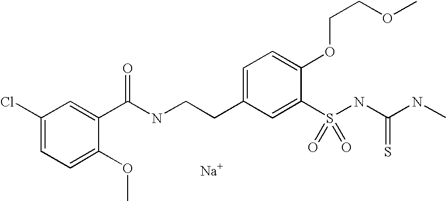 |
| 3-(1,2,2-Trimethylpropylamino)- 4H-pyrido(2,3-e][1,2,4]thiadiazine 1,1-dioxide | C12H18N4O2S | | | K(ATP) Channel Activators | 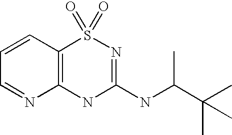 |
| 5-Chloro-2-methoxy-N-[2-[4- methoxy-3-(3- methylthioureidosulfonyl)phenyl]ethyl]benzamide sodium salt | C19H21ClN3NaO5S2 | Clamikalant sodium | | K(ATP) Channel Blockers | 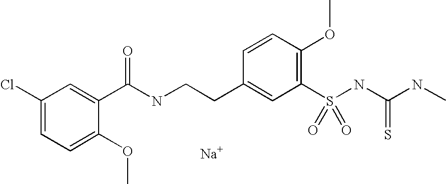 |
| 3-(2,4-Dichloro-6- methylbenzylamino)-4-(1,1- dimethylpropylamino)-3- cyclobutene-1,2-dione | C17H20Cl2N2O2 | | | K(ATP) Channel Activators | 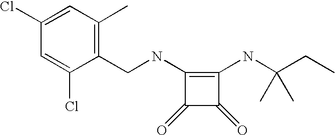 |
| 4-[2-(tert-Butylamino)-3,4-dioxo-1- cyclobutenylaminomethyl]-3- chlorobenzonitrile | C16H16ClN3O2 | | | K(ATP) Channel Activators | 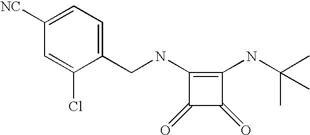 |
| 3-Chloro-4-[2-(1,1-dimethylpropyl- amino)-3,4-dioxo-1- cyclobutenylaminomethyl]benzonitrile | C17H18ClN3O2 | | | K(ATP) Channel Activators | 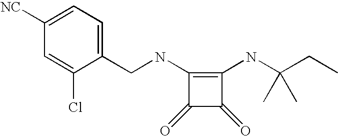 |
| 9-(3,4-Dichlorophenyl)-3,3,6,6- tetramethyl-1,2,3,4,5,6,7,8,9,10- decahydroacridine-1,8-dione | C23H25Cl2NO2 | | | K(ATP) Channel Blockers | 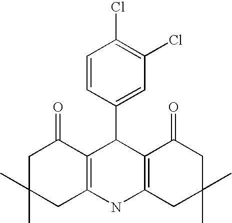 |
| 6-Chloro-N-isopropyl-4H- thieno[3,2-e][1,2,4]thiadiazin-3- amine 1,1-dioxide | C8H10ClN3O2S2 | | | K(ir) 6.2/SUR1 Activators | 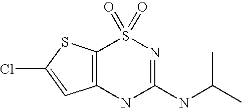 |
| 4-[5-[N2-Cyano-N3-(1,2,2- trimethylpropyl)guanidino]-2- (difluoromethoxy)phenyl]-2,6- dimethyl-1,4-dihydropyridine-3,5- dicarboxylic acid dimethyl ester | C26H33F2N5O5 | | | Calcium Channel Blockers; K(ATP) Channel Activators | 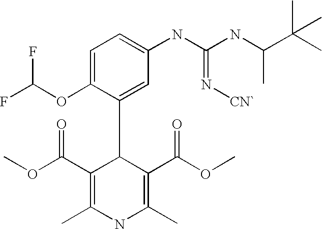 |
| 1-(3-Chloro-5-cyanophenyl)-2- cyano-3-(1,1- diethylpropyl)guanidine | C16H20ClN5 | | | K(ATP) Channel Activators | 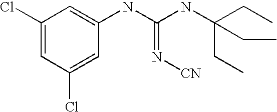 |
| 2-Cyano-1-(3,5-dichlorophenyl)-3- (1,1-diethylpropyl)guanidine | C15H20Cl2N4 | | | K(ATP) Channel Activators | 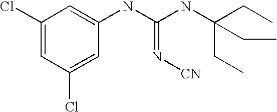 |
| 5-Chloro-2-methoxy-N-[7-methoxy- 6-(N′-methylthioureidosulfonyl)- 3,4-dihydro-2H-1-benzopyran-3- yl]benzamide | C20H22ClN3O6S2 | | | K(ATP) Channel Blockers | 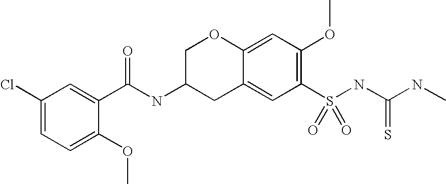 |
| 5-Chloro-2-methoxy-N-[7-methoxy- 6-(N′-methylureidosulfonyl)-3,4- dihydro-2H-1-benzopyran-3- yl]benzamide | C20H22ClN3O7S | | | K(ATP) Channel Blockers | 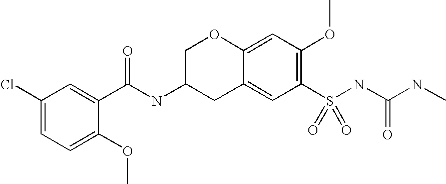 |
| 5-Chloro-2-methoxy-N-[7-methoxy- 6-(N′-propylureidosulfonyl)-3,4- dihydro-2H-1-benzopyran-3- yl]benzamide | C22H26ClN3O7S | | | K(ATP) Channel Blockers | 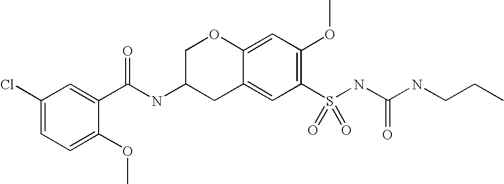 |
| 5-Chloro-N-[6-(N′- ethylthioureidosulfonyl)-7-methoxy- 3,4-dihydro-2H-1-benzopyran-3- yl]-2-methoxybenzamide | C21H24ClN3O6S2 | | | K(ATP) Channel Blockers | 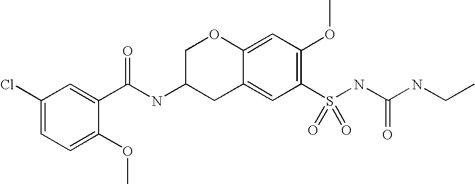 |
| 5-Fluoro-N-[6-(N′- isopropylaminothioureidosulfonyl)- 7-methoxy-2,2-dimethyl-3,4- dihydro-2H-1-benzopyran-3-yl]-2- methoxybenzamide | C24H30FN3O6S2 | | | K(ATP) Channel Blockers | 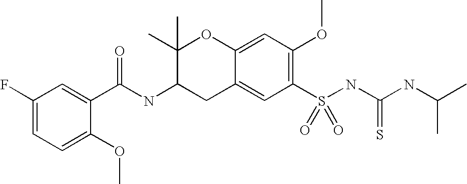 |
| trans-4-(4-Methylcyclohexyl)-4- oxobutanoic acid | C11H18O3 | | | Insulin Secretagogues; K(ATP) Channel Blockers | 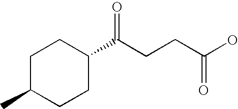 |
| (3S,4R)-3-Hydroxy-4-[2(S)- (hydroxymethyl)-5-oxopyrrolidinyl]- 2,2-dimethyl-3,4-dihydro-2H-1- benzopyran-6-carbonitrile | C17H20N2O4 | | | K(ATP) Channel Activators | 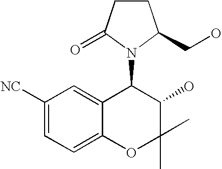 |
| 6-Chloro-3-(1-methylheptylamino)- 4H-thieno[3,2-e][1,2,4]thiadiazine- 1,1-dioxide | C13H20ClN3O2S2 | | | K(ATP) Channel Activators | 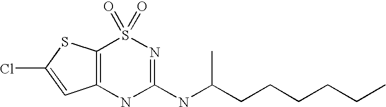 |
| 6-Chloro-3-(2-methylbutylamino)- 4H-thieno[3,2-e][1,2,4]thiadiazine- 1,1-dioxide | C10H14ClN3O2S2 | | | K(ATP) Channel Activators | 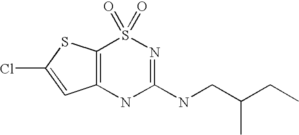 |
| 4-[3,4-Dioxo-2-[1(R),2,2- trimethylpropylamino]-1- cyclobuten-1-ylamino]-3- ethylbenzonitrile | C19H23N3O2 | | | K(ATP) Channel Activators | 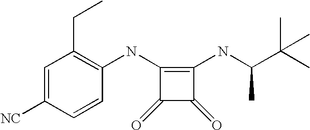 |
| 2-[3-(2-Methoxyethoxy)-7-(4- methylphenyl)naphthalen-2-yl]-4,5- dihydro-1H-imidazole | C23H24N2O2 | | | Insulin Secretagogues; K(ATP) Channel Blockers | 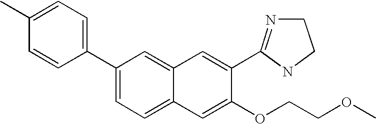 |
| 2-[4-(4-Chlorophenyl)-3-(2- methoxyethoxy)naphthalen-2-yl]- 4,5-dihydro-1H-imidazole | C22H21ClN2O2 | | | Insulin Secretagogues; K(ATP) Channel Blockers | 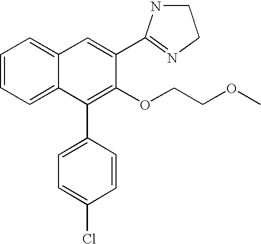 |
| (+)-9(R)-(3-Bromo-4-fluorophenyl)- 2,3,4,5,6,7,8,9-octahydrothieno[3,2- b]quinoline-8-one 1,1-dioxide | C17H15BrFNO3S | | | K(ATP) Channel Activators | 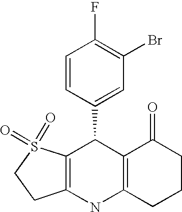 |
| 6-Chloro-3-isopropoxy-4H- thieno[3,2-e]-1,2,4-thiadiazine 1,1- dioxide | C8H9ClN3O3S2 | | | K(ATP) Channel Activators | 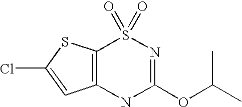 |
| 6-Chloro-3-(cyclopentyloxy)-4H- thieno[3,2-e]-1,2,4-thiadiazine 1,1- dioxide | C10H11ClN2O3S2 | | | K(ATP) Channel Activators | 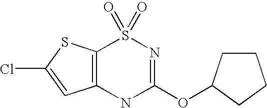 |
| 6-Chloro-3-(cyclopropylmethyl- sulfanyl)-4H-thieno[3,2-e]-1,2,4- thiadiazine 1,1-dioxide | C9H9ClN2O2S3 | | | K(ATP) Channel Activators | 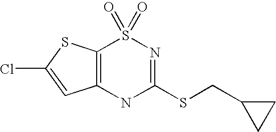 |
| 6-Chloro-3-(ethylsulfanyl)-4H- thieno[3,2-e]-1,2,4-thiadiazine- 1,1 dioxide | C7H7ClN2O2S3 | | | K(ATP) Channel Activators | 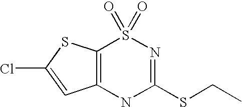 |
| 6-Chloro-3-(isopropylsulfanyl)-4H- thieno[3,2-e]-1,2,4-thiadiazine 1,1-dioxide | C8H9ClN2O2S3 | | | K(ATP) Channel Activators | 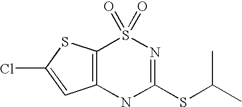 |
| 6-Chloro-3-(propylsulfanyl)-4H- thieno[3,2-e]-1,2,4-thiadiazine 1,1-dioxide | C8H9ClN2O2S3 | | | K(ATP) Channel Activators | 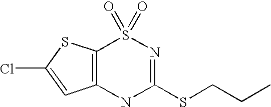 |
| 6-Chloro-3-(cyclopentylsulfanyl)- 4H-thieno[3,2-e]-1,2,4-thiadiazine 1,1-dioxide | C10H11ClN2O2S3 | | | K(ATP) Channel Activators | 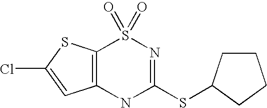 |
| 6-Chloro-3-(1-methylpropyl)-4H- thieno[3,2-e]-1,2,4-thiadiazine 1,1(2H)-dioxide | C9H11ClN2O2S3 | | | K(ATP) Channel Activators | 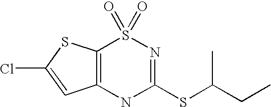 |
| 6-Chloro-3-(isobutylsulfanyl)-4H- thieno[3,2-e]-1,2,4-thiadiazine 1,1-dioxide | C9H11ClN2O2S3 | | | K(ATP) Channel Activators | 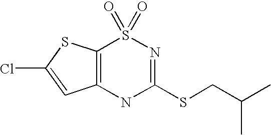 |
| 6-Chloro-3-(propylsulfinyl)-4H- thieno[3,2-e]-1,2,4-thiadiazine 1,1-dioxide | C8H9ClN2O3S3 | | | K(ATP) Channel Activators | 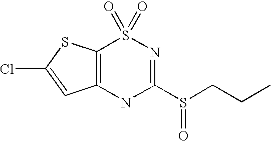 |
| 6-Chloro-3-methoxy-4H-thieno[3,2- e]-1,2,4-thiadiazine 1,1-dioxide | C6H5ClN3O3S2 | | | K(ATP) Channel Activators | 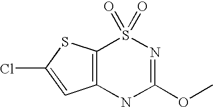 |
| 3-(2-Butylamino)-4H-pyrido[4,3-e]- 1,2,4-thiadiazine 1,1-dioxide | C10H14N4O2S | | | K(ATP) Channel Activators | 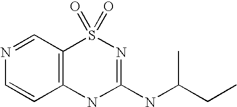 |
| (3S,4R)-N-(4-Chlorophenyl)-N′- cyano-N″-[6-(diisobutylsulfamoyl)- 3-hydroxy-2,2-dimethyl-3,4-dihydro- 2H-1-benzopyran-4-yl] | | | | | |
| # guanidine; (3S,4R)-4-[N″-(4-Chlorophenyl)- N′-cyanoguanidino]-3-hydroxy- N,N-bis(isobutyl)-2,2-dimethyl-3,4- dihydro-2H-1-benzopyran-6- sulfonamide | C27H36ClN5O4S | | | K(ATP) Channel Activators | 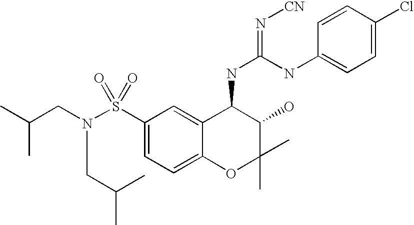 |
| (3S,4R)-3-Hydroxy-2,2-dimethyl-4- (2-oxopiperidin-1-yl)-N-phenyl-1- benzopyran-6-sulfonamide | C22H26N2O5S | | | K(ATP) Channel Activators | 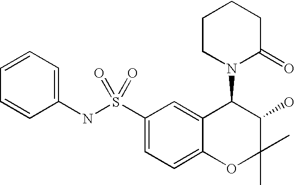 |
| N-[3,5-Bis(trifluoromethyl)phenyl]- N′-cyano-N″-(3- methylbutyl)guanidine | C15H16F6N4 | | | K(ir) 6.2/SUR1 Activators | 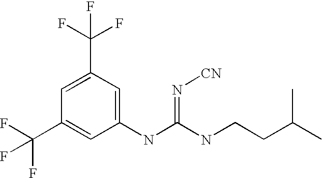 |
| trans-4-[2(S)-(1-Ethoxyethoxy- methyl)-5-oxopyrrolidin-1-yl]-3- hydroxy-2,2-dimethyl-3,4-dihydro- 2H-1-benzopyran-6-carbonitrile | C21H28N2O5 | | | K(ATP) Channel Activators | 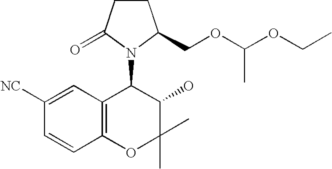 |
| 3-(1,2-Dimethylpropylamino)-4H- pyrido[2,3-e][1,2,4]thiadiazine 1,1-dioxide | C11H16N4O2S | | | K(ATP) Channel Activators | 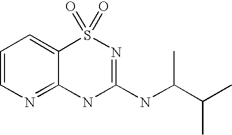 |
| 6-Chloro-3-(1,2-dimethylpropyl- amino)-4H-pyrido[2,3-e][1,2,4]thiadiazine1,1-dioxide | C11H15ClN4O2S | | | K(ATP) Channel Activators | 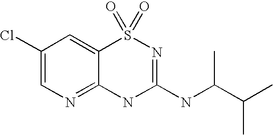 |
| 7-Chloro-3-(1,2,2-trimethylpropyl- amino)-4H-pyrido[2,3-e][1,2,4]thiadiazine 1,1-dioxide | C12H17ClN4O2S | | | K(ATP) Channel Activators | 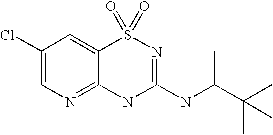 |
| 4(R)-[(1R,6S)-4-(2-Chlorobenzyl)- 5-oxo-3,4-diazabicyclo[4.1.0]hept- 2-en-2-yloxy]-3(S)-hydroxy-2,2- dimethyl-3,4-dihydro-2H-1- benzopyran-6-carbonitrile | C24H22ClN3O4 | | | K(ATP) Channel Activators | 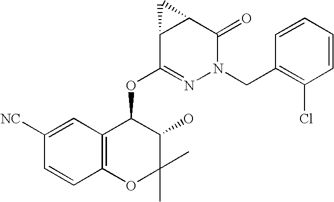 |
| 9(R)-(4-Fluoro-3-iodophenyl)- 2,3,5,4,8,9-hexahydro-4H- pyrano[3,4-b]thieno[2,3-e]pyridine-1,1,8-trione | C16H13FlNO4S | | | K(ATP) Channel Activators | 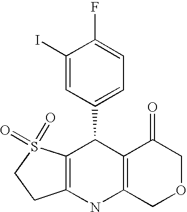 |
| (+)-9(R)-(3-Bromo-4-fluorophenyl)- 3,4,5,7,8,9-hexahydro-2H- pyrano[3,4-b]thieno[2,3-e]pyridine-1,1,8-trione | C16H13BrFNO4S | | | K(ATP) Channel Activators | 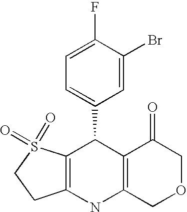 |
| 3-[3,5-Bis(trifluoromethyl) phenylamino]-2-(4-chlorophenyl- sulfonyl)-3-(1,2,2-trimethyl- propylamino)-2(E)-propenenitrile | C23H22ClF6N3O2S | | | K(ATP) Channel Activators | 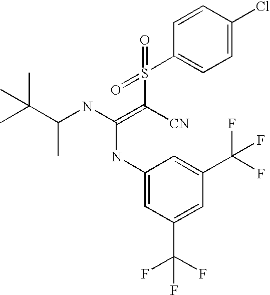 |
| 2-(4-Chlorophenylsulfonyl)-3-(3,5- dimethoxyphenylamino)-3-(1,2,2- trimethylpropylamino)-2(E)- propenenitrile | C23H28ClN3O4S | | | K(ATP) Channel Activators | 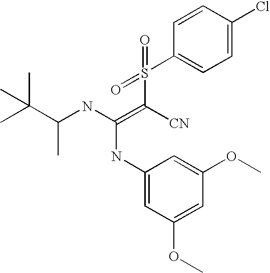 |
| 3-[3,5-Bis(trifluoromethyl) phenylamino]-2-(4-chlorophenyl- sulfonyl)-3-[1(S),2,2-trimethyl- propylamino]-2(E)-propenenitrile | C23H22ClF6N3O2S | | | K(ATP) Channel Activators | 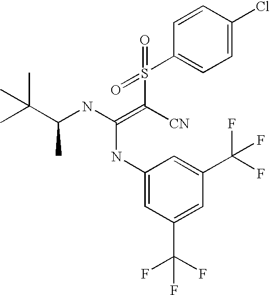 |
| 2-(4-Chlorophenylsulfonyl)-3-(3,5- dimethoxyphenylamino)-3-(1,1- dimethylpropylamino)-2(E)- propenenitrile | C22H26ClN3O4S | | | K(ATP) Channel Activators | 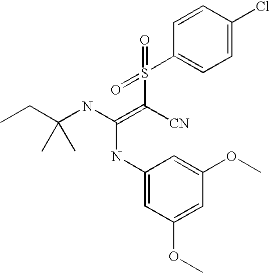 |
| 3-[3-Fluoro-5-(trifluoromethyl) phenylamino]-2-(methylsulfonyl)- 3-(1,2,2-trimethylpropyl- amino)-2(E)-propenenitrile | C17H21F4N3O2S | | | K(ATP) Channel Activators | 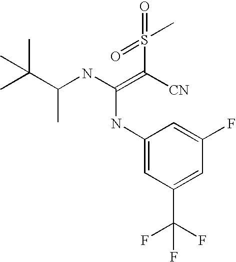 |
| 3-[3,5-Bis(trifluoromethyl)phenyl- amino]-3-(cyclopentylamino)- 2-(isopropylsulfonyl)-2-(E)- propenenitrile | C19H21F6N3O2S | | | K(ATP) Channel Activators | 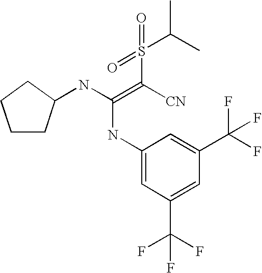 |
| 3-(5-Benzodioxolylamino)-3-(1,1- dimethylpropylamino)-2- (methylsulfonyl)-2(E)- propenenitrile | C16H21N3O4S | | | K(ATP) Channel Activators | 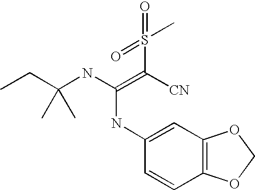 |
| 3-(tert-Butylamino)-6-chloro-4H- thieno[3,2-e][1,2,4]thiadiazine-1,1-dioxide | C9H12ClN3O2S2 | | | K(ir) 6.2/SUR1 Activators | 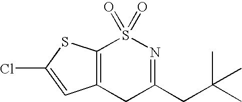 |
| 6-Chloro-3-(1,1-dimethylpropyl- amino)-4H-thieno[3,2-e][1,2,4]thiadiazine-1,1-dioxide | C10H14ClN3O2S2 | | | K(ATP) Channel Activators | 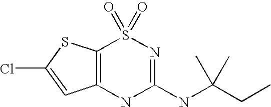 |
| 6-Chloro-3-(1-methylcyclopropyl- amino)-4H-thieno[3,2-e][1,2,4]thiadiazine-1,1-dioxide | C9H10ClN3O2S2 | Tifenazoxide | | Apoptosis Inhibitors; K(ir) 6.2/SUR1 Activators | 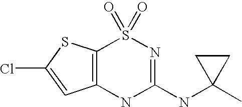 |

Preferred embodiments of the invention are described in the following Example. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the example.

### EXAMPLE

### Activation of Hypothalamic ATP-Sensitive Potassium Channels is Necessary and Sufficient for Suppression of Liver Glucose Production

Example Summary

Diabetic hyperglycemia is due to increased rates of gluconeogenesis (Rothman et al., 1991; Magnusson et al., 1992). Here we show that activation of K_{ATP} channels (Anguilar-Bryan et al., 1995) within the medial hypothalamus is per se sufficient to lower blood glucose levels via inhibition of hepatic gluconeogenesis. Furthermore, the administration of insulin within the medial hypothalamus lowers blood glucose levels via K_{ATP} channel dependent inhibition of gluconeogenesis. Consistent with these results, SUR1 null mice (Seghers et al., 2000) display a selective resistance to the inhibitory action of insulin on gluconeogenesis. Finally, surgical resection of the hepatic branch of the vagus nerve negates the effects of central insulin on the hepatic expression of gluconeogenic enzymes and on gluconeogenesis. These findings indicate that activation of hypothalamic K_{ATP} channels normally restrains hepatic gluconeogenesis and any alteration within this CNS/liver circuit can contribute to diabetic hyperglycemia.

Materials and Methods

Animal preparation for the in vivo experiments. Rats. Eighty-nine 10-week-old male Sprague-Dawley rats (Charles River Breeding Laboratories, Wilmington, Mass.) were studied. Rats were housed in individual cages and subjected to a standard light-dark cycle. Three weeks prior to the in vivo studies, chronic catheters were implanted in the third cerebral ventricle (Obici et al., 2003; Liu et al., 1998) or intrahypothalamically (Morton et al., 2003) (IH) by stereotaxic surgery. One week before the pancreatic-insulin clamp protocols, rats received additional catheters in the right internal jugular and left carotid artery (FIG. 5) (Obici et al., 2003; Liu et al., 1998).

Mice. Adult male SUR1KO4 (n=8) and wild-type (WT, n=8) littermate mice (27-32 g) were anesthetized with chloral hydrate (400 mg/kg bw i.p.) and catheterized through the right internal jugular vein as previously described (Obici et al., 2003; Liu et al., 1998). The venous catheter was used for infusions, and blood samples were collected from the tail vein.

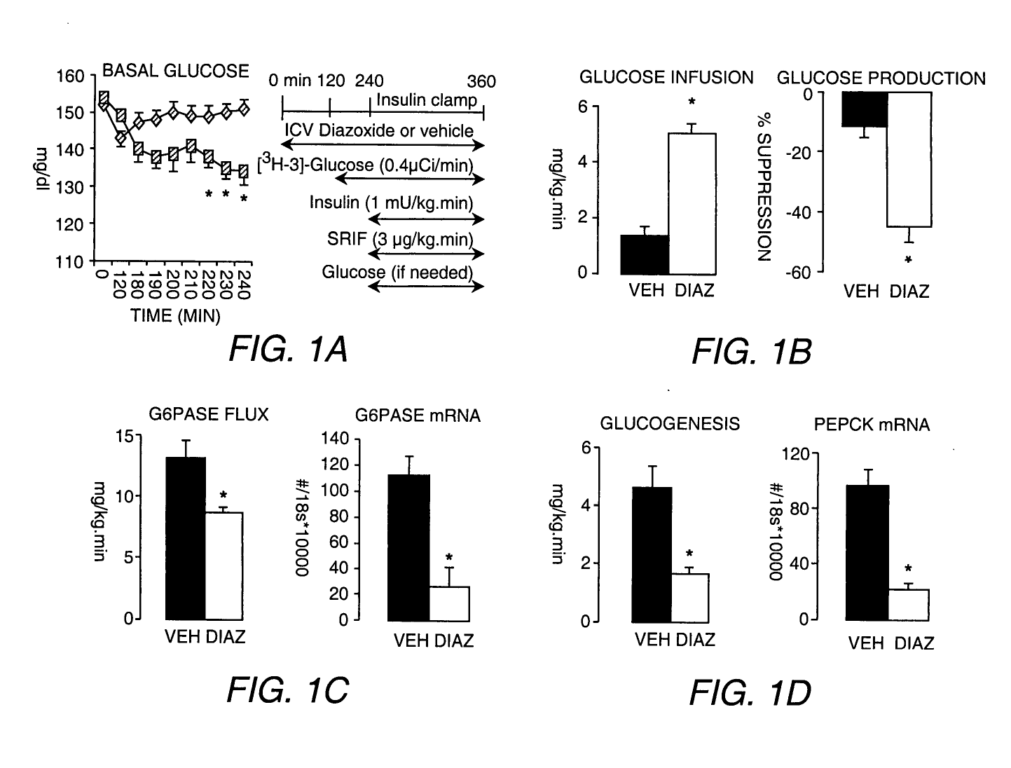

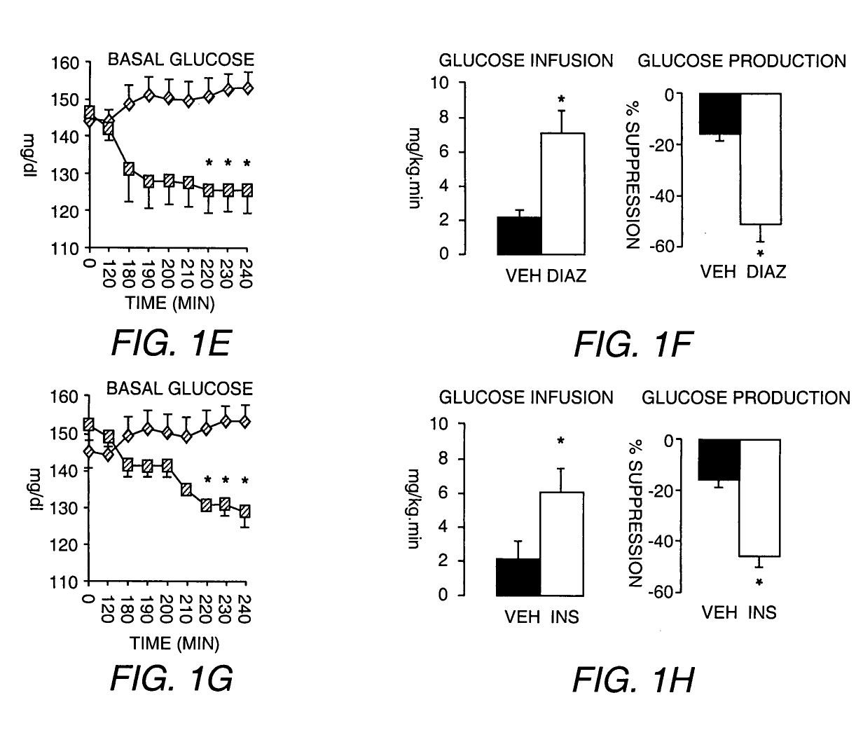

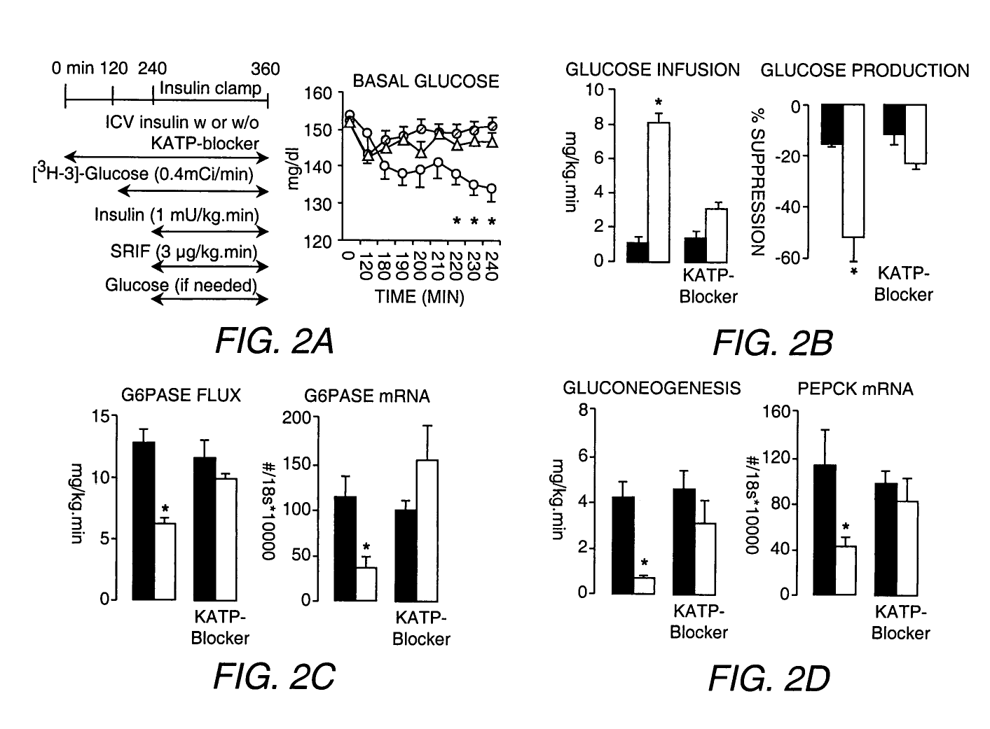

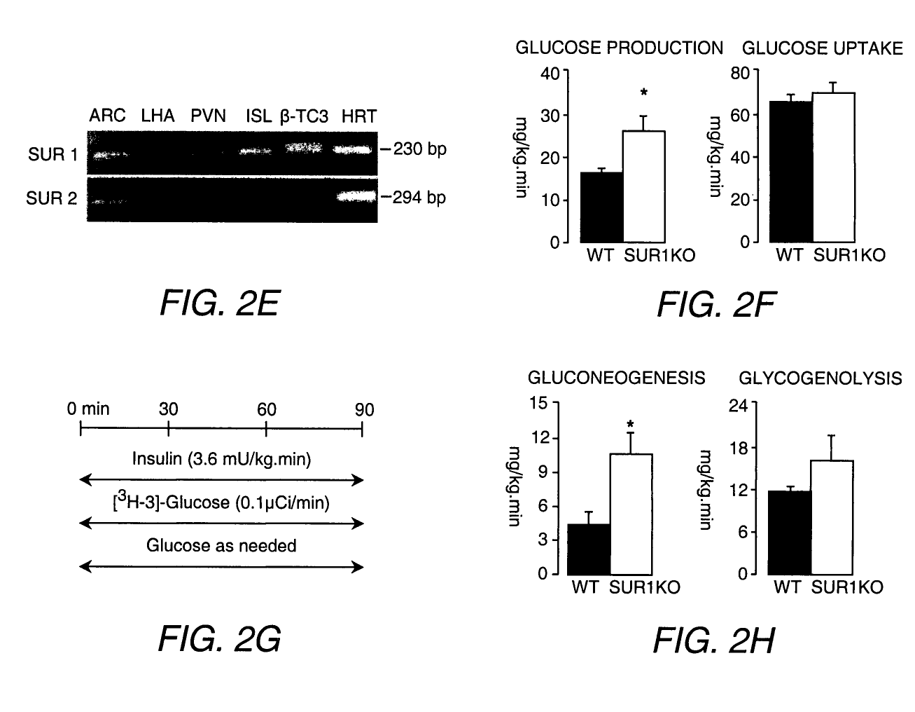

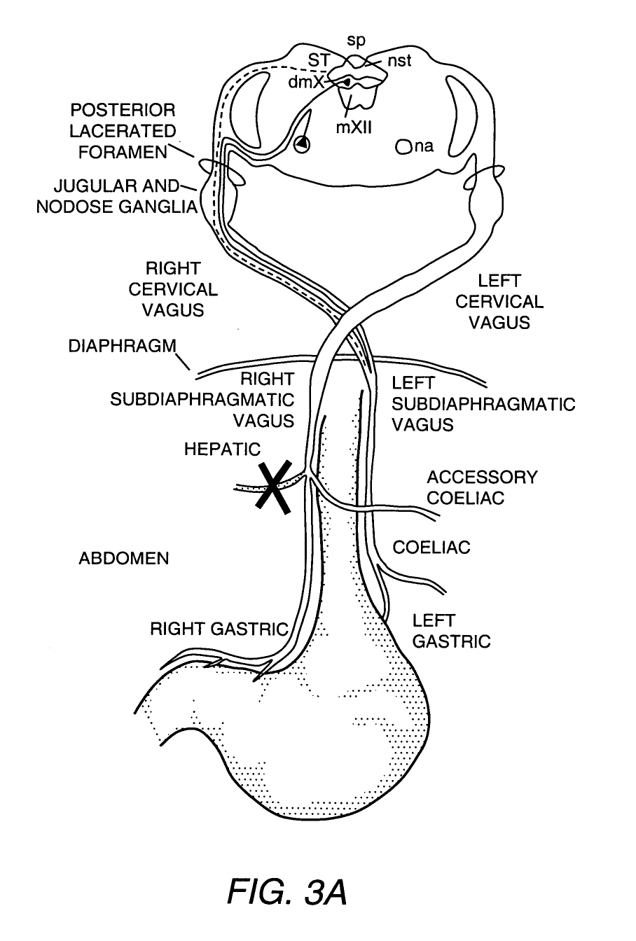

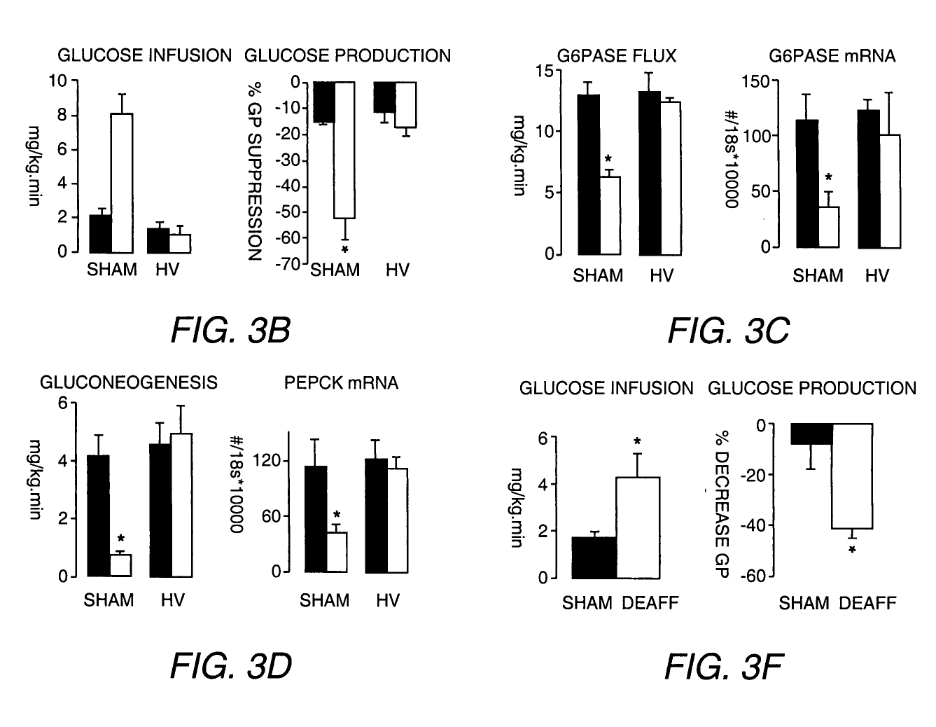

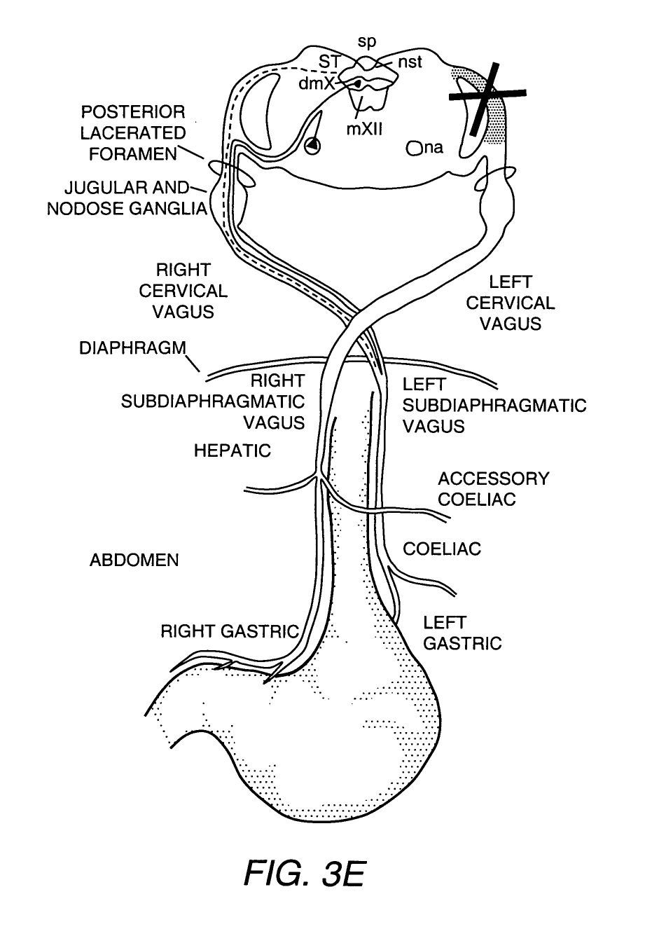

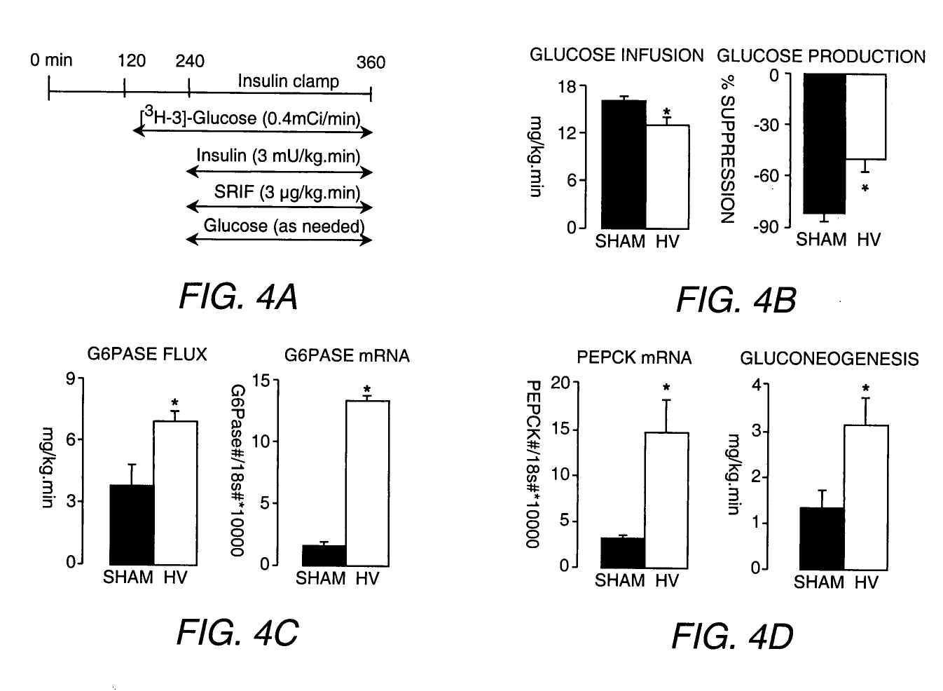

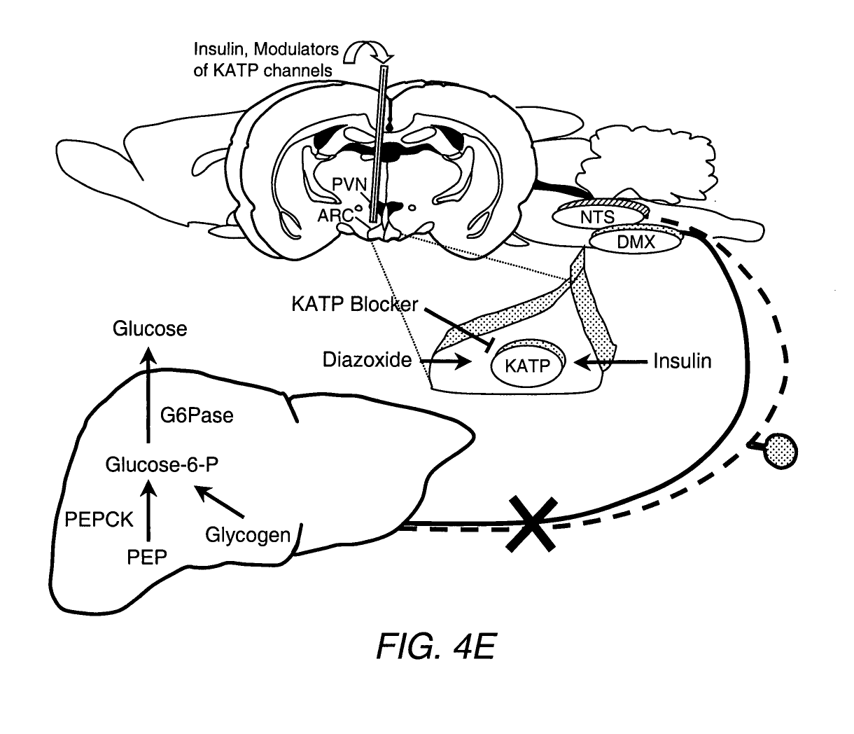

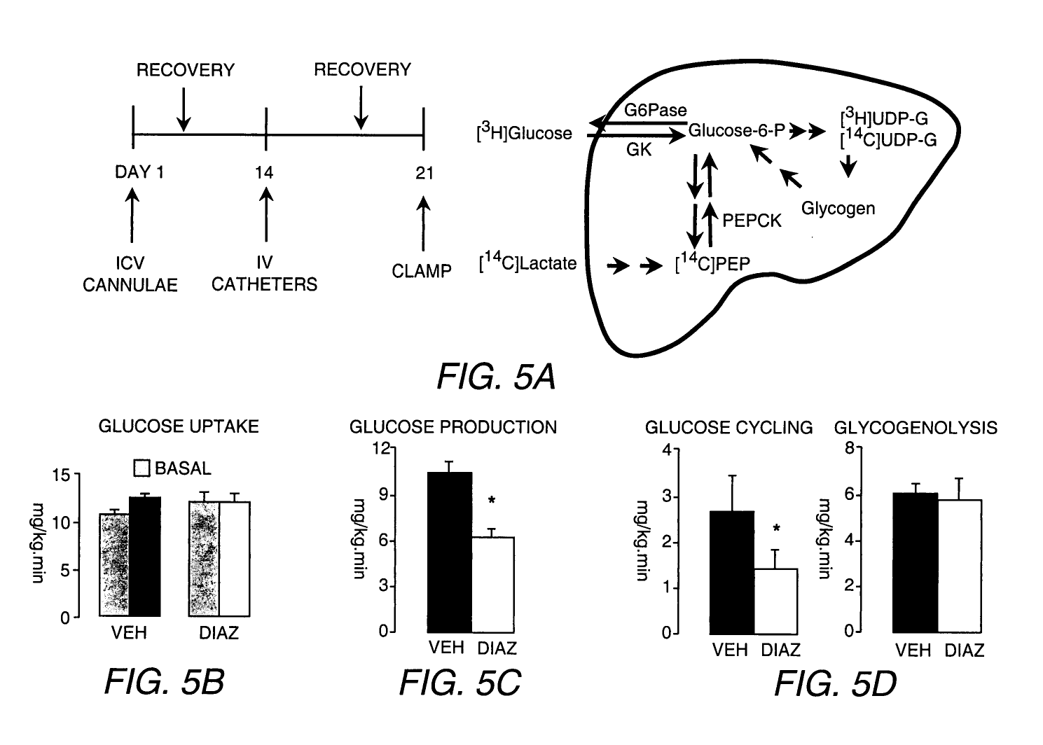

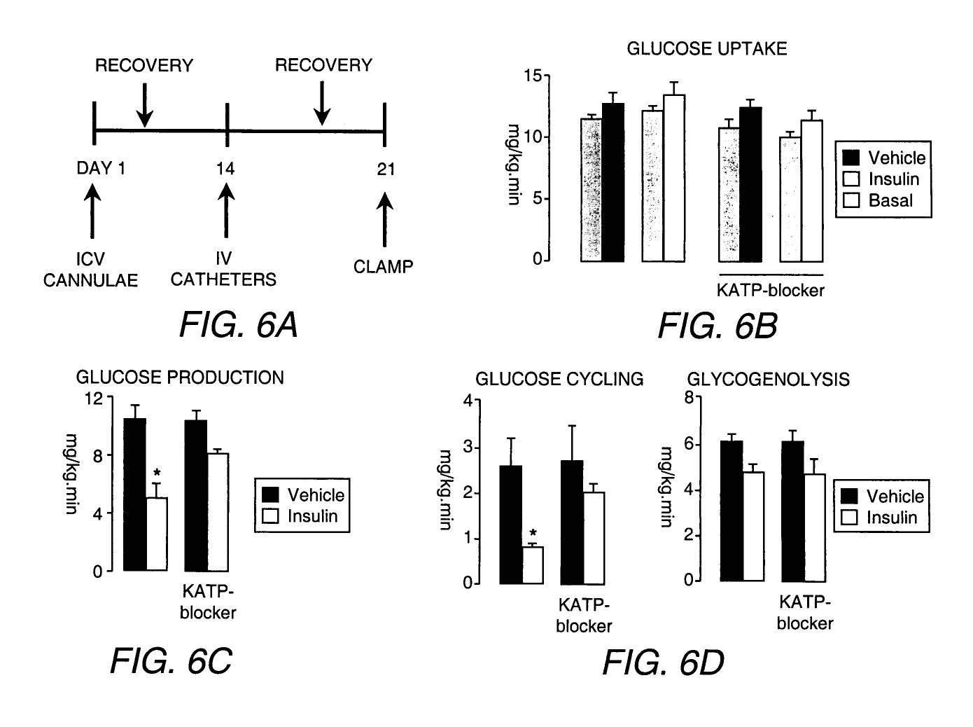

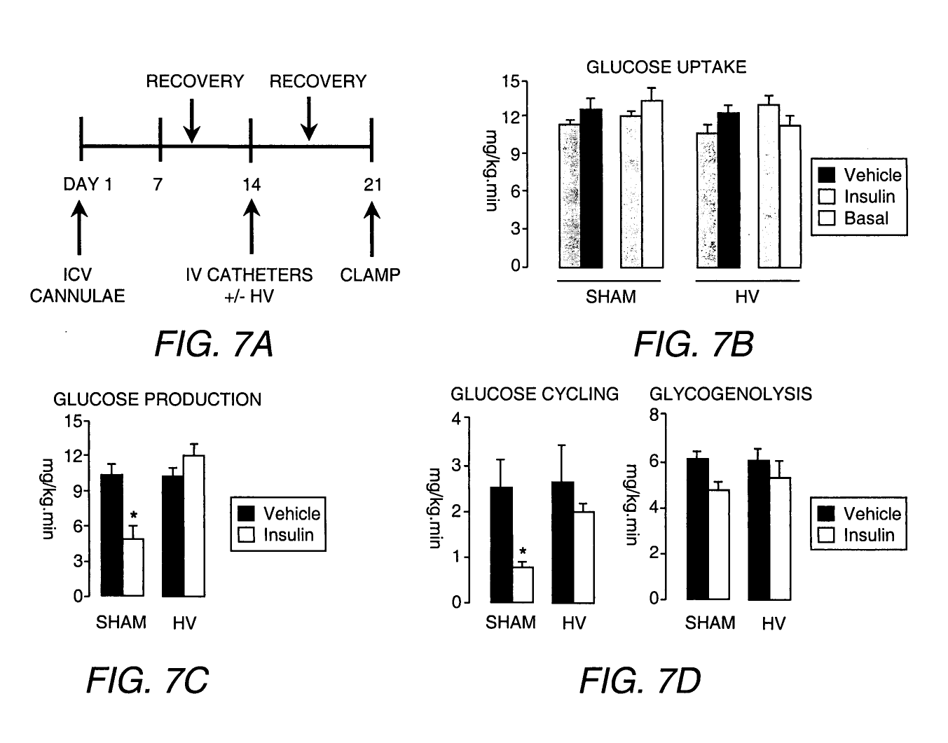

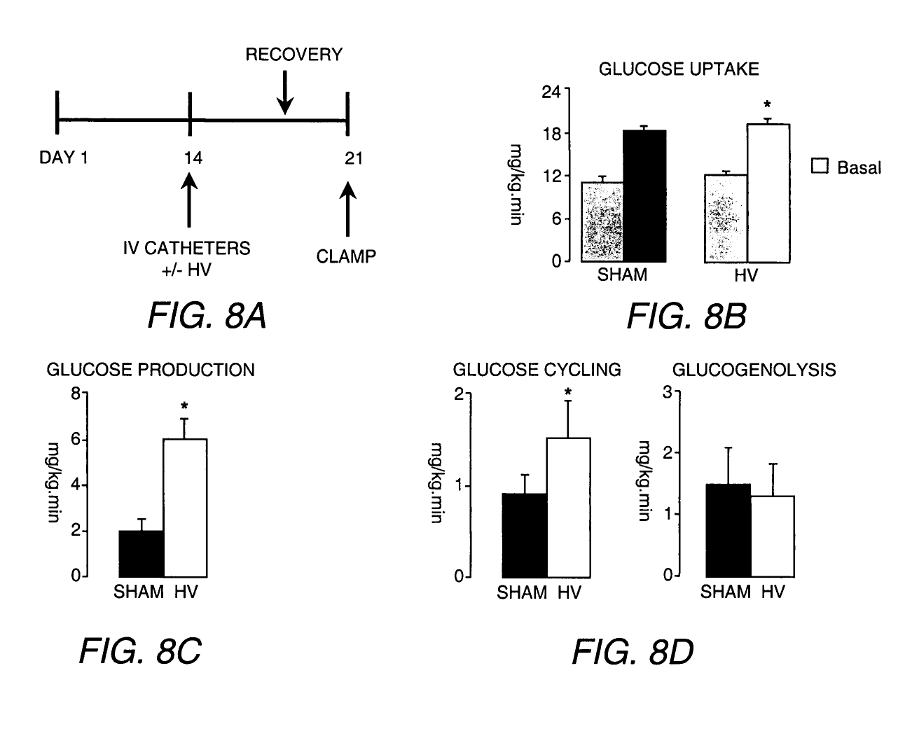

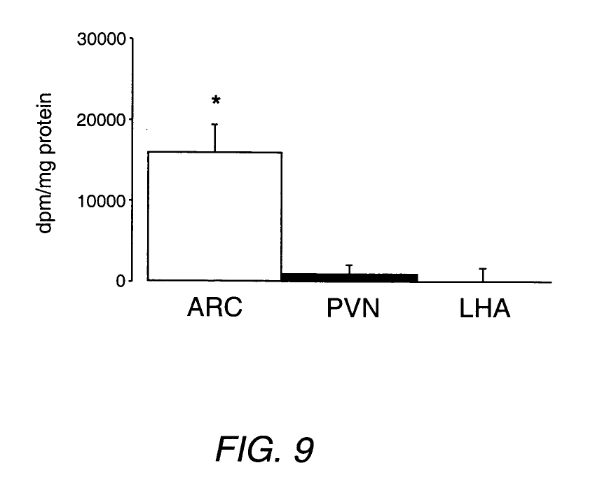

In vivo studies. For the studies in rats, the metabolic experiments were performed ˜3 weeks after stereotaxic surgery following complete recovery from the operation (FIG. 5). All solutions were diluted with artificial cerebral spinal fluid (aCSF). At t=0 a primed-continuous ICV infusion of either insulin (Ins, 30 μU total dose), glibenclamide (K_{ATP} channel blocker, 3 nmol), diazoxide (Diaz, 1.5 nmol) or vehicles was initiated and maintained for the remainder of the study (FIG. 1A). In separate experiments, insulin or diazoxide was infused IH with similar time course at the total dose of 2 μU and 100 pmol respectively. Pancreatic-insulin clamp studies were performed as previously described (Liu et al., 1998). Euglycemic-hyperinsulinemic clamps in conscious, unrestrained, catheterized mice were performed for 90 min as previously described (Obici et al., 2003; Liu et al., 1998).

Selective hepatic branch vagotomy. One week before the clamp procedure a subgroup of rats underwent selective hepatic vagotomy (HV) or sham operation (SHAM). Anatomical nerve transactions was verified in each rat at sacrifice by microscopic observation of the absence of vagal nerve fibers in between the two silk sutures that had been placed along the trunk at the time of transection (la Fleur et al., 2003).

Selective vagal deafferentation. Nerve transection was performed as previously described, according to the methods of Norgren and Smith (Norgren & Smith, 1998).

Gene expression. Gluconeogenic enzymes. Quantitative analysis of gene expression was done using RT-PCR. Total RNA was isolated with Trizol (Invitrogen) and single-strand cDNA was synthesized with a kit provided by Invitrogen. Real-time PCR reactions and the primers for G6 Pase and PEPCK were done as described (Pocai et al., in press). The copy number of each transcript was derived from a standard curve of cloned target templates. Expression of each transcript was normalized to copy number for 18s ribosomal protein.

Detection of Sur1 and Sur2 mRNA in hypothalamic nuclei. PCR with primer sets specific for either rat SUR1 (forward, 5′CTTCCAGACAGCGAGGGAGAA3′ [SEQ ID NO:1]); reverse,

5′TCTCCATGGGGCAGGATGTCT3′ [SEQ ID NO:2]) or SUR2 (forward,

5′ATGAGCCTTTCCTTCTGTGGT3′ [SEQ ID NO:3]; reverse,

5′CGCCCGTTGCGAGTCTGAAACA3′ [SEQ ID NO:4]) was performed on cDNA synthesized from total RNA extracted from rat arcuate (ARC), lateral hypothalamus (LHA) and paraventricular nucleus (PVN). Expression of SUR1 and SUR2 was compared to that of other tissues, including rat pancreatic islets (ISL), mouse beta-TC-3 cells (BTC-3) and rat heart (HRT). The resulting PCR products were-resolved in a 2% agarose gel and visualized after staining with ethidium bromide. The expected size for the PCR products is 230 basepairs (bp) for SUR1 and 294 bp.

Brain stereotactic ‘micropunches’. Brain ‘micropunches’ of individual hypothalamic nuclei were prepared as described before (Obici et al., 2003).

Hepatic alucose fluxes. Rate of hepatic glucose fluxes was determined as described. Barzilai et al., 1997).

All values are presented as the means +/−SE. Comparisons among groups were made using analysis of variance or unpaired students t test as appropriate. The study protocol was reviewed and approved by the Institutional Animal Care and Use Committee of the Albert Einstein College of Medicine.

Results and Discussion

To investigate whether direct activation of central K_{ATP} channels is sufficient to reproduce the potent effects of insulin on blood glucose levels, on endogenous glucose production (GP), and on hepatic gluconeogenesis, the K_{ATP} channel activator diazoxide was infused in the third cerebral ventricle of conscious rats. Central administration of diazoxide lowered blood glucose levels (FIG. 1A). To examine the mechanisms by which central activation of K_{ATP} channels decreases blood glucose we combined intracerebroventricular (ICV) infusions with systemic pancreatic-insulin clamp studies (FIG. 1A). Paired groups of rats received ICV infusions of vehicle or diazoxide (FIG. 1A). In the presence of basal circulating insulin levels, glucose infusion was required to prevent hypoglycemia following central administration of diazoxide (FIG. 1B). We next assessed glucose kinetics by tracer dilution methodology in order to establish whether the increased requirement for glucose infusion in response to central activation of K_{ATP} channels is due to stimulation of glucose uptake or to inhibition of GP. In the presence of basal insulin levels (˜20 μU/ml), ICV diazoxide markedly and significantly decreased GP (FIG. 1B) (by 45±4%), while the rate of glucose uptake was not significantly affected by ICV treatments (FIG. 5B). Thus, central stimulation of K_{ATP} channels per se lowers blood glucose via inhibition of GP. GP represents the net contribution of glucosyl units derived from gluconeogenesis and glycogenolysis. However, a portion of glucose entering the liver via phosphorylation of extracellular glucose is also a substrate for de-phosphorylation via glucose-6-phosphatase (G6 Pase) creating a futile cycle named glucose cycling (FIG. 5D). In order to further delineate the mechanisms by which central activation of K_{ATP} channels modulates glucose homeostasis we estimated the in vivo flux through G6 Pase and the relative contribution of gluconeogenesis and glycogenolysis to glucose output. ICV diazoxide decreased the flux through G6 Pase (FIG. 1C) in parallel to its effects on GP (FIG. 1B). Importantly, the decrease in GP was largely accounted for by marked inhibition of gluconeogenesis (FIG. 1D) while the rate of glycogenolysis was not significantly decreased (FIG. 5D). Based on these in vivo results, we next assessed the effect of central activation of K_{ATP} channels on G6 Pase and PEPCK expression in liver harvested at the completion of the infusions. Real time PCR analyses revealed that ICV diazoxide markedly decreased liver G6 Pase and PEPCK mRNA levels (FIG. 1C,D). Thus, direct activation of central K_{ATP} channels was per se sufficient to recapitulate the action of insulin on the in vivo fluxes through G6 Pase and gluconeogenesis and on the hepatic expression of the catalytic subunit of G6 Pase and PEPCK. These potent metabolic effects of diazoxide could be mediated by its activation of K_{ATP} channels anywhere within the CNS (Grill et al., 2002). To gain insight into the anatomical localization of these effects we next infused a fifteen-fold lower dose of diazoxide (FIG. 1E,F) bilaterally within the parenchyma of the medial hypothalamus. Placement of the cannulae was verified by infusion of radioactive tracers followed by sampling of hypothalamic nuclei by micropunches (FIG. 9). Intrahypothalamic (IH) infusion of diazoxide lowered blood glucose levels (FIG. 1E). This hypoglycemic effect was due to marked suppression of GP (FIG. 1F) in the presence of basal circulating levels of insulin. In order to examine whether the effects of ICV insulin on liver glucose homeostasis (Obici et al., 2002a) are also centered in an overlapping hypothalamic area, we next infused insulin (FIGS. 1G,H) bilaterally within the parenchyma of the medial hypothalamus. The IH infusions of insulin at a dose fifteen-fold lower than that previously used in ICV experiments (Obici et al., 2002a) reproduced the potent effects of ICV insulin or diazoxide on blood glucose concentration (FIG. 1G) and on GP (FIG. 1H). Thus, activation of either K_{ATP} channels or of insulin signaling within the medial hypothalamus is sufficient to decrease blood glucose levels via suppression of GP, hepatic gluconeogenesis, and PEPCK and G6 Pase expression. These ‘gain-of-function’ experiments suggest that modulation of K_{ATP} channels activity within the medial hypothalamus can have a major impact on liver glucose homeostasis.

Sulfonylureas (K_{ATP} channel-blockers) are potent inhibitors of K_{ATP} channels and they block the activation of hypothalamic K_{ATP} channels by insulin and leptin (Spanswick et al., 1997; 2000). Here, we show that the central administration of insulin (in the third cerebral ventricle) also lowers blood glucose levels and that this effect requires central activation of K_{ATP} channels (FIG. 2A). To investigate the mechanisms by which central insulin decreases blood glucose we combined ICV infusions with systemic pancreatic-insulin clamp studies (FIG. 2A). Paired groups of rats received ICV infusions of vehicle, insulin, insulin and K_{ATP} channels blocker, or K_{ATP} channel blocker alone (FIG. 2A). In the presence of near basal circulating insulin levels, glucose infusion was required to prevent hypoglycemia following central administration of insulin. ICV infusion of the K_{ATP} channels blocker glibenclamide alone did not modify the rate of glucose infusion compared with ICV vehicle (FIG. 2B). Importantly, central administration of insulin did not alter glucose requirements when the K_{ATP} channel blocker was co-infused. In the presence of basal and equal insulin levels (−20 μU/ml), ICV insulin markedly and significantly decreased GP (FIG. 2B) (by 52±8%). The decrease in GP was negated by the ICV co-infusion of K_{ATP} channels blocker and completely accounted for the effect of central insulin on whole body glucose metabolism. On the basis of these results, we conclude that central stimulation of insulin signaling results in K_{ATP} channel-dependent suppression of GP. In order to further delineate the mechanisms by which central insulin modulates glucose homeostasis we estimated the in vivo flux through G6 Pase and the relative contribution of gluconeogenesis and glycogenolysis to glucose output. As shown in FIG. 2C, ICV insulin markedly decreased the flux through G6 Pase in parallel to its effects on GP (FIG. 2B). The decrease in glucose output was largely accounted for by a marked inhibition of gluconeogenesis (FIG. 2D) while the rate of glycogenolysis was not significantly decreased (FIG. 6D). Importantly, in the presence of ICV sulfonylurea, central insulin did not modify hepatic glucose fluxes. As observed with the central activation of K_{ATP} channels, real time PCR analyses revealed that central stimulation of insulin action resulted in a marked K_{ATP} channels dependent decreases in liver G6 Pase and PEPCK mRNA levels (FIGS. 2C,D). Thus, the decreases in the in vivo flux through G6 Pase and gluconeogenesis induced by the ICV administration of insulin appears to be largely mediated by the decreased hepatic expression of the catalytic subunit of G6 Pase and PEPCK. The latter effects were completely prevented by ICV infusion of the K_{ATP} channel blocker.

Genetic and electro-physiological evidence indicate the presence of K_{ATP} channels in selective hypothalamic neurons. These channels have an octameric structure similar to that of peripheral K_{ATP} channels with a K⁺ inward rectifier subunit, K_{IR} 6.1 or 6.2, and a sulfonylurea receptor SUR-1 or SUR-2 (Aguilar-Bryan et al., 1995; Aguilar-Bryan and Bryan, 1999). To gain insight into the composition of K_{ATP} channels within the hypothalamus we designed primers specific for the SUR1 or SUR2 genes and investigated their expression in key hypothalamic nuclei. SUR1 and SUR2 mRNA are both detectable in the medial hypothalamus (arcuate nuclei) (FIG. 2E). Hypothalamic neurons expressing K_{ATP} channels are targets of insulin (Spanswick et al., 2000) and their high sensitivity to diazoxide and low concentrations of sulfonylurea (Seino and Miki, 2003) suggests that SUR1 is a component of these insulin-responsive K_{ATP} channels (Aguilar-Bryan et al., 1995; Inagaki et al., 1996). Lack of SUR1-containing K_{ATP} channels has been shown to lead to an increase in the membrane potential that cannot be suppressed by diazoxide (Seghers et al., 2000). Since our findings in rats suggest that the neuronal hyperpolarization induced by the hypothalamic administration of insulin or diazoxide modulates liver glucose homeostasis, we next investigated whether SUR1-containing K_{ATP} channels are required for this effect. More specifically we asked whether insulin's ability to restrain hepatic gluconeogenesis is selectively impaired in SUR1 null (SUR1 KO) mice. To this end, we performed insulin clamp studies in conscious SUR1 KO and wild type (WT) mice (FIG. 2G). SUR1 KO displayed hepatic but not peripheral insulin resistance (FIG. 2F). In the presence of physiological hyperinsulinemia, the rate of glucose production was increased by ˜2 fold in SUR1 KO compared with WT mice. This increase in GP was largely due to a marked increase in the rate of gluconeogenesis while glycogenolysis was not significantly altered (FIG. 2H). Thus, a selective impairment in insulin action on gluconeogenesis is a feature of SUR1 KO mice. We postulate that insulin activation of SUR1 containing K_{ATP} channels within the hypothalamus is required to restrain hepatic gluconeogenesis. This is the first demonstration of a defect in hepatic insulin action in SUR1 null mice and it stands in contrast with the increased insulin sensitivity reported in Kir6.2 and SUR2 null mice (Seino et al., 2000; Chutkow et al., 2001). Taken together with the results of ‘gain-of-function’ experiments, these pharmacological and genetic ‘loss-of-function’ experiments in rats and mice respectively indicate that SUR1-containing K_{ATP} channels within the medial hypothalamus are likely to play an important role in the regulation of liver glucose homeostasis.

Hypothalamic centers participate in the short-term regulation of ingestive behavior via descending neural connections to the caudal brainstem leading to activation of vagal input to the gastrointestinal tract (Schwartz et al., 2000; Grill et al., 2002). Since autonomic neural input to the liver can also rapidly modulate liver metabolism (Matsuhisa et al., 2000), we next asked whether central administration of insulin decreases GP and the expression of G6 Pase and PEPCK via activation of hepatic efferent vagal fibers. To this end, we tested the effects of the central administration of insulin in rats with selective hepatic branch vagotomy (HV) or sham-operation (SHAM) (FIG. 3A). Paired groups of SHAM and HV rats received an ICV infusion of either insulin or vehicle. In the presence of ˜basal circulating insulin levels, glucose infusion was required to prevent hypoglycemia following ICV insulin in the SHAM but not in the HV rats (FIG. 3B). This vagus-dependent effect of central insulin could be due to stimulation of glucose uptake or to suppression of GP. The rate of glucose uptake was not significantly affected by ICV treatments in either SHAM or HV rats (FIG. 7B). Conversely, in the presence of basal and equal insulin levels, ICV insulin markedly and significantly decreased GP (by 48±3%) in SHAM but not in HV rats (FIG. 3C). On the basis of these results, we conclude that the inhibition of GP in response to central stimulation of insulin action requires an intact hepatic branch of the vagus nerve. We next estimated the in vivo flux through G6 Pase and the relative contribution of gluconeogenesis and glycogenolysis (FIG. 7D) to glucose output. As shown in FIG. 3C, ICV insulin markedly decreased the flux through G6 Pase in the SHAM rats. The decrease in GP was largely accounted for by a marked inhibition of gluconeogenesis (FIG. 3D). Importantly, in rats with hepatic branch vagotomy, central insulin failed to affect hepatic glucose fluxes. Real time PCR analyses showed that ICV insulin resulted in a marked decrease in liver G6 Pase and PEPCK mRNA levels in SHAM but not in HV rats (FIGS. 3C,D). Thus, the marked decreases in the in vivo flux through G6 Pase and PEP-gluconeogenesis and in the hepatic expression of the catalytic subunit of G6 Pase and PEPCK following central administration of insulin were negated by hepatic branch vagotomy (FIGS. 3C,D).

The hepatic branch of the vagus nerve is comprised of efferent and afferent fibers and its resection abolished the hepatic effects of ICV insulin. In this regard, it is conceivable that metabolic changes primarily induced within the liver could generate signals that are conveyed up the afferent hepatic branch of the vagus to the brainstem, in turn eliciting activation of the descending vagal fibers (Moore et al., 2002). Thus, in order to address the potential role of redundant hepatic branch vagal fibers in mediating the hypoglycemic effects of ICV insulin, we performed additional experiments in animals with selective vagal deafferentation. These animals have intact descending efferent fibers to the liver but all their vagal afferents supplying the hepatic vagal branch are resected at the site of entry in the brainstem (FIG. 3E). Vagal deafferentation did not alter the ability of central insulin to lower blood glucose levels and to suppress GP (FIG. 3F). Thus, the efferent vagal input to the liver is required for the inhibition of glucose production following central administration of insulin while the afferent input from the hepatic branch of the vagus nerve to the brainstem is not required.

These findings indicate that the activation of hypothalamic insulin receptors results in marked suppression of hepatic gluconeogenesis and that this central effect of insulin requires activation of K_{ATP} channels within the medial hypothalamus and efferent vagal input to the liver. We next investigated whether this central pathway of insulin action plays a role in the overall effects of circulating insulin on liver glucose homeostasis. To estimate the contribution of the hepatic branch of the vagus to the physiological effects of insulin on the liver, we generated physiological increases in circulating insulin levels in SHAM and HV rats using the pancreatic-insulin clamp technique (FIG. 4A). Plasma insulin levels were increased by ˜3-fold over basal (Table 4) in order to submaximally stimulate glucose disposal and inhibit glucose production. In the presence of similar increases in plasma insulin levels, higher rates of glucose infusion were required in SHAM compared with HV in order to prevent hypoglycemia (FIG. 4b). Consistent with this finding, hyperinsulinemia resulted in a marked suppression of GP (80±8%) in SHAM, which was severely impaired in HV (38±11%; FIG. 4). Thus, hepatic branch vagotomy leads to a loss of ˜half of the inhibitory action of circulating insulin on hepatic glucose production. We next estimated the in vivo flux through G6 Pase and the relative contribution of gluconeogenesis and glycogenolysis (FIG. 8B) to glucose output. The flux through G6 Pase (FIG. 4C) and gluconeogenesis (FIG. 4D) were higher in HV compared with SHAM. Consistent with the role of the hepatic vagal branch in the regulation of these enzymes, real time PCR analyses showed that the hepatic expression of G6 Pase and PEPCK was increased in HV rats compared with SHAM (FIGS. 4C,D). Thus, hepatic branch vagotomy also interferes with the inhibitory effects of the systemic administration of insulin on the in vivo flux through G6 Pase and gluconeogenesis and on the hepatic expression of the catalytic subunit of G6 Pase and PEPCK (FIGS. 4C,D).

The following tables provide supplemental data from relevant experiments.

TABLE 2
Modulation of KATP channels: general characteristics of the
experimental groups before and during the pancreatic/insulin
(1 mu/kg.min) clamp studies.

| | Veh | DIAZ |
|---|---|---|
| N | 4 | 6 |
| Basal: | | |
| Body wt. (g) | 311 ± 6 | 306 ± 4 |
| Glucose (mM) | 8.2 ± 0.6 | 7.9 ± 0.4 |
| FFA (mM) | 0.5 ± 0.2 | 0.4 ± 0.1 |
| Clamp | | |
| Glucose (mM) | 8.1 ± 0.5 | 301 ± 4 |
| Insulin (ng/ml) | 1.0 ± 0.3 | 1.2 ± 0.4 |
| FFA (mM) | 0.6 ± 0.2 | 0.5 ± 0.1 |

Data are means ± SE. The values during the clamp represent steady-state levels obtained by averaging at least four plasma samples during the experimental period. Body weight was measured at the beginning of the clamp. FFA, free fatty acids.

TABLE 3
Modulation of KATP channels and hypothalamic insulin action: general
characteristics of the experimental groups before and during the
pancreatic/insulin (1 mu/kg.min) clamp studies.

| | Veh | INS | Gly | INS + Gly |
|---|---|---|---|---|
| N | 5 | 7 | 5 | 5 |
| Basal: | | | | |
| Body wt.(g) | 304 ± 1 | 300 ± 5 | 316 ± 8 | 301 ± 4 |
| Glucose (mM) | 8.4 ± 0.2 | 7.8 ± 0.3 | 8.2 ± 0.5 | 8.0 ± 0.5 |
| FFA (mM) | 0.6 ± 0.1 | 0.5 ± 0.3 | 0.4 ± 0.1 | 0.5 ± 0.2 |
| Clamp: | | | | |
| Glucose (mM) | 8.2 ± 0.8 | 7.6 ± 0.5 | 8.2 ± 0.9 | 8.0 ± 0.6 |
| Insulin (ng/ml) | 1.2 ± 0.2 | 1.3 ± 0.2 | 1.2 ± 0.5 | 1.1 ± 0.3 |
| FFA (mM) | 0.5 ± 0.2 | 0.4 ± 0.1 | 0.6 ± 0.2 | 0.5 ± 0.3 |

Data are means ± SE. The values during the clamp represent steady-state levels obtained by averaging at least four plasma samples during the experimental period. Body weight was measured at the beginning of the clamp. FFA, free fatty acids.

TABLE 3A
SURI KO mice: general characteristics of the experimental groups
before and during the pancreatic/insulin (3.6 mU/kg.min) clamp studies.

| | WT | SUR1 KO |
|---|---|---|
| N | 8 | 6 |
| Body wt. (g) | 29.9 ± 0.9 | 32.1 ± 1.3 |
| Glucose (mM) | 8.2 ± 0.4 | 7.9 ± 0.3 |
| Insulin (ng/ml) | 4.3 ± 0.9 | 4.9 ± 1.6 |
| Leptin (ng/ml) | 3.5 ± 1.5 | 3.0 ± 0.7 |
| Glucagon (pM) | 147 ± 6.7 | 138 ± 10.5 |
| Resistin (pg/ml) | 444 ± 40 | 433 ± 126 |
| FFA (mM) | 0.6 ± 0.1 | 0.5 ± 0.3 |

Data are means ± SE. The values represent steady-state levels obtained during the experimental period. Body weight was measured at the beginning of the clamp. FFA, free fatty acids.

TABLE 4
Hypothalamic insulin action and hepatic vagotomy: general
characteristics of the experimental groups before and during the
pancreatic/insulin (1 mU/kg.min) clamp studies.

| | Veh | INS | Veh | INS |
|---|---|---|---|---|
| | Sham | | HV | |
| N | 6 | 5 | 5 | 6 |
| Basal | | | | |
| Body wt.(g) | 301 ± 7 | 310 ± 5 | 308 ± 8 | 295 ± 4 |
| Glucose (mM) | 8.2 ± 0.4 | 7.9 ± 0.3 | 8.1 ± 0.5 | 7.9 ± 0.5 |
| FFA (mM) | 0.6 ± 0.1 | 0.5 ± 0.3 | 0.4 ± 0.1 | 0.5 ± 0.2 |
| Clamp | | | | |
| Glucose (mM) | 8.2 ± 0.8 | 7.6 ± 0.5 | 8.2 ± 0.9 | 8.0 ± 0.6 |
| Insulin (ng/ml) | 1.1 ± 0.2 | 1.2 ± 0.2 | 1.0 ± 0.5 | 0.9 ± 0.3 |
| FFA (mM) | 0.5 ± 0.2 | 0.4 ± 0.1 | 0.6 ± 0.2 | 0.5 ± 0.3 |

Data are means ± SE. The values during the clamp represent steady-state levels obtained by averaging at least four plasma samples during the experimental period. Body weight was measured at the beginning of the clamp. FFA, free fatty acids.

TABLE 5
Insulin action and hepatic vagotomy: general characteristic of the
experimental groups before and during the pancreatic/insulin
(3 mU/Kg.min) clamp studies.

| | Sham | HV |
|---|---|---|
| N | 13 | 13 |
| Basal. | | |
| Body wt. (g) | 306 ± 4 | 296 ± 5 |
| Glucose (mM) | 8.2 ± 0.5 | 8.1 ± 0.3 |
| FFA (mM) | 0.5 ± 0.2 | 0.4 ± 0.1 |
| Clamp | | |
| Glucose (mM) | 8.0 ± 0.6 | 8.1 ± 0.5 |
| Insulin (ng/ml) | 2.9 ± 0.4 | 3.2 ± 0.4 |
| FFA (mM) | 0.6 ± 0.2 | 0.5 ± 0.1 |

Data are means ± SE. The values during the clamp represent steady-state levels obtained by averaging at least four plasma samples during the experimental period. Body weight was measured at the beginning of the clamp. FFA, free fatty acids.

TABLE 6
Modulation of KATP channels: Specific activities of hepatic
substrates used to calculate the direct pathway and the “indirect pathway”.

| | Veh | DIAZ |
|---|---|---|
| N | 4 | 7 |
| ³H-Glucose Plasma SA (dpm/nmol) | 45.5 ± 2.2 | 46.0 ± 14.2 |
| ³H-UDPglucose Liver SA (dpm/nmol) | 7.60 ± 0.7 | 6.79 ± 1.6 |
| Direct (%) | 16.9 ± 1.7 | 14.37 ± 2.2 |
| ¹⁴C-PEP(dpm/nmol) | 6.8 ± 1.4 | 11.60 ± 2.4 |
| ¹⁴C-UDP glucose (dpm/nmol) | 7.6 ± 0.7 | 3.85 ± 0.3 |
| % Indirect | 42.6 ± 6.1 | 9.7 ± 4.1* |

Data are means ± SE.
*p < 0.05 as compared with respective vehicle treated animals.

TABLE 7
Modulation of KATP channels and Hypothalamic Insulin Action:
Specific activities of hepatic substrates used to calculate the
direct pathway and the “indirect pathway”.

| | | | | INS + |
|---|---|---|---|---|
| | | | K_{ATP}- | K_{ATP}- |
| | Veh | INS | Blocker | Blocker |
| N | 5 | 7 | 6 | 5 |
| ³H-Glucose | 3.5 ± 1.4 | 43.1 ± 1.9 | 48.8 ± 3.9 | 52.4 ± 3.5 |
| Plasma SA | | | | |
| (dpm/nmol) | | | | |
| ³H-UDP- | 7.5 ± 0.6 | 5.5 ± 0.5 | 8.7 ± 0.6 | 6.1 ± 0.5 |
| glucose Liver | | | | |
| SA (dpm/nmol) | | | | |
| Direct (%) | 17.4 ± 1.4 | 12.1 ± 0.7 | 18.5 ± 2.8 | 11.8 ± 1.0 |
| 14C-PEP | 6.7 ± 1.1 | 10.9 ± 0.6 | 8.8 ± 2.4 | 5.5 ± 1.4 |
| (dpm/nmol) | | | | |
| 14C-UDP | 4.5 ± 0.4 | 2.2 ± 0.1 | 5.3 ± 1.0 | 2.7 ± 0.3 |
| glucose | | | | |
| (dpm/nmol) | | | | |
| % Indirect | 45.3 ± 5.0 | 11.6 ± 1.0* | 32.4 ± 3.0 | 34.4 ± 9.7 |

Data are means ± SE.
*p < 0.05 as compared with respective vehicle treated animals.

TABLE 7A
SUR1KO mice: Specific activities of hepatic substrates used to
calculate the direct pathway and the “indirect Pathway”.

| | WT | SUR1 KO |
|---|---|---|
| N | 8 | 5 |
| ³H-Glucose Plasma SA (dpm/nmol) | 19.2 ± 1.1 | 20.2 ± 2.2 |
| ³H-UDPglucose Liver SA (dpm/nmol) | 1.0 ± 0.1 | 0.8 ± 0.1 |
| Direct (%) | 4.5 ± 0.9 | 4.2 ± 0.4 |
| ¹⁴C-PEP (dpm/nmol) | 12.5 ± 3.5 | 4.8 ± 1.4 |
| ¹⁴C-UDP glucose (dpm/nmol) | 6.0 ± 1.1 | 3.5 ± 1.2 |
| % Indirect | 25.1 ± 4.1 | 39.3 ± 6.2* |

Data are means ± SE.
*p < 0.05 as compared with wild type mice (WT).

TABLE 8
Insulin action and hepatic vagotomy: Specific activities of hepatic
substrates used to calculate the direct pathway and
the “indirect pathway”.

| | VEH | ICV INS | VEH | ICV INS |
|---|---|---|---|---|
| | SHAM | | HV | |
| N | 5 | 7 | 6 | 5 |
| ³H-Glucose | 43.5 ± 1.4 | 43.1 ± 1.9 | 44.9 ± 3.9 | 52.4 ± 3.5 |
| Plasma SA | | | | |
| (dpm/nmol) | | | | |
| ³H-UDP- | 7.5 ± 0.6 | 5.5 ± 0.5 | 6.6 ± 0.5 | 6.1 ± 0.5 |
| glucose Liver | | | | |
| SA (dpm/nmol) | | | | |
| Direct (%) | 17.4 ± 1.4 | 12.1 ± 0.71 | 4.5 ± 1.7 | 11.8 ± 1.0 |
| ¹⁴C-PEP | 6.3 ± 1.1 | 10.9 ± 0.6 | 6.8 ± 3.2 | 5.5 ± 1.4 |
| (dpm/nmol) | | | | |
| ¹⁴C-UDP | 4.5 ± 0.4 | 2.2 ± 0.1 | 4.7 ± 1.2 | 2.7 ± 0.3 |
| glucose | | | | |
| (dpm/nmol) | | | | |
| % Indirect | 45.3 ± 5.0 | 11.6 ± 1.0* | 47.4 ± 15.4 | 34.4 ± 9.7 |

Data are means ± SE.
*p< 0.05 ascompared with respective vehicle treated animals.

TABLE 9
Insulin action and hepatic vagotomy: Specific activities of hepatic
substrates used to calculate the direct pathway and
the “indirect pathway”.

| | SHAM | HV |
|---|---|---|
| | (3 mU) | |
| N | 10 | 6 |
| ³H-Plasma SA (dpm/nmol) | 29.9 ± 2.5 | 28.6 ± 1.8 |
| ³H-UDPglucose Liver SA (dpm/nmol) | 6.3 ± 0.9 | 6.3 ± 0.7 |
| Direct (%) | 22.8 ± 3.9 | 22.0 ± 2.3 |
| ¹⁴C-PEP (dpm/nmol) | 7.2 ± 1.4 | 5.1 ± 1.1 |
| ¹⁴C-UDP glucose (dpm/nmol) | 3.8 ± 0.6 | 5.3 ± 0.7 |
| % Indirect | 34.5 ± 6.7 | 60.7 ± 9.4* |

Data are means ± SE.
*p < 0.05 as compared with respective vehicle treated animals.

Taken together with the presence of hepatic insulin resistance in SUR1 null mice, these results in HV rats establish a physiological role for this insulin-driven brain-liver circuit. In fact, our findings indicate that the neuronal circuit engaged in response to the activation of hypothalamic insulin signaling also plays an important role in restraining hepatic gluconeogenesis in response to physiological increases in the circulating insulin levels. Since increased gluconeogenesis is the main cause of fasting hyperglycemia in DM2 (Magnusson et al., 1992) and reversing liver insulin resistance is a prominent goal of diabetic therapy, improving hypothalamic insulin signaling is likely to become a novel therapeutic strategy for this disease. There is increasing evidence for nutritional and hormonal signals acting on the medial hypothalamus to curtail both exogenous and endogenous input of nutrients (Loftus et al., 2000; Minokoshi et al., 2004; Obici et al., 2002c). The apparent similarities between the brain-liver neural circuit activated in response to changes in hypothalamic lipid oxidation (Obici et al., 2003; 2002c) and in response to activation of hypothalamic insulin signaling suggest a convergence of these nutritional signals perhaps at the level of hypothalamic K_{ATP} channels.

Our findings demonstrate that activation of K_{ATP} channels as well as stimulation of insulin signaling within the medial hypothalamus is sufficient to lower blood glucose levels via rapid inhibition of hepatic gluconeogenesis. Conversely, insulin action on liver glucose homeostasis requires activation of hypothalamic K_{ATP} channels and efferent vagal input to the liver. Consistent with this notion SUR1 null mice display a selective impairment in insulin action on hepatic gluconeogenesis. Thus, bi-directional changes in the activity of hypothalamic K_{ATP} channels lead to rapid changes in circulating glucose levels via modulation of autonomic input to the liver. This novel and important role of neuronal K_{ATP} channels in the regulation of glucose homeostasis adds to their well-established role in the regulation of β-cell function (Aguilar-Bryan et al., 1995). Thus, K_{ATP} channels are likely to play a fundamental and pleiotropic role in the modulation of blood glucose levels with their activation in the hypothalamus decreasing hepatic gluconeogenesis and their activation in pancreatic β-cells decreasing insulin secretion. The central and peripheral actions of K_{ATP} channels may be designed to balance each other in order to maintain glucose homeostasis. Any disruption in the equilibrium between these regulatory circuits is likely to result in altered glucose homeostasis.

Additional-experiments were also performed in rats maintained on a high fat diet regimen for three days. This protocol rapidly induces severe hepatic insulin resistance (glucose production is not readily suppressed by insulin). In the presence of basal and similar plasma concentrations of insulin (20 μU/ml), the central administration of insulin failed to significantly inhibit glucose production in this model. However, the ICV or intrahypothalamic infusion of diazoxide effectively inhibited glucose production by ˜45%. These studies demonstrate that the activation of hypothalamic K_{ATP} channels can bypass the impairment in hypothalamic insulin action in this model.

Overall, the experiments described herein demonstrate for the first time that the activation of K_{ATP} channels within the mediobasal hypothalamus is sufficient to lower blood glucose levels via a marked suppression of liver gluconeogenesis. Since an increase in liver gluconeogenesis is the main cause of fasting hyperglycemia in patients with diabetes mellitus, pharmacological activation of these channels with drugs such as diazoxide could be an effective therapy for this metabolic disorder.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

1. A method of reducing peripheral blood glucose levels in a mammal, the method comprising intranasally administering a K_{ATP} channel activator to the hypothalamus of the mammal in an amount effective to reduce peripheral blood glucose levels in the mammal.

2. The method of claim 1, wherein the K_{ATP} channel activator is a substituted guanidine or a benzothiazine 1,1-dioxide.

3. The method of claim 1, wherein the K_{ATP} channel activator is selected from the group consisting of diazoxide, pinacidil, (−)-cromakalim, aprikalim, bimakalim, emakalim, nicordandil, NNC 55-0118, NN414, EMD55387, HOE234, KRN2391, diaminonitroethane, minoxidil sulfate, P1060, P1075, RP49356, RP66471, and any combination thereof.

4. The method of claim 1, wherein the K_{ATP} channel activator is diazoxide.

5. The method of claim 1, wherein the mammal has at least one condition selected from the group consisting of obesity, type 2 diabetes, type 1 diabetes, hyperglycemia, insulin resistance, glucose intolerance, leptin resistance, metabolic syndrome, gonadotropin deficiency, amenorrhea, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension and polycystic ovary syndrome.

6. The method of claim 1, wherein the mammal is a human.

7. The method of claim 1, wherein the K_{ATP} channel activator has a molecular weight of 50,000 daltons or less.

8. The method of claim 1, wherein the method comprises intranasally administering a pharmaceutical composition comprising the K_{ATP} channel activator and a pharmaceutically acceptable carrier.

9. The method of claim 8, wherein the pharmaceutical composition is an aqueous solution.

10. The method of claim 9, wherein the aqueous solution is selected from the group consisting of an aqueous gel, an aqueous suspension, an aqueous microsphere suspension, an aqueous microsphere dispersion, an aqueous liposomal dispersion, aqueous micelles of liposomes, an aqueous microemulsion, and any combination of the foregoing.

11. The method of claim 8, wherein the pharmaceutical composition is a nonaqueous solution.

12. The method of claim 11, wherein the nonaqueous solution is selected from the group consisting of a nonaqueous gel, a nonaqueous suspension, a nonaqueous microsphere suspension, a nonaqueous microsphere dispersion, a nonaqueous liposomal dispersion, a nonaqueous emulsion, a nonaqueous microemulsion, and any combination of the foregoing.

13. The method of claim 8, wherein the pharmaceutical composition is a powder formulation.

14. The method of claim 13, wherein the powder formulation is selected from the group consisting of a simple powder mixture, a micronized powder, powder microspheres, coated powder microspheres, and any combination of the foregoing.

15. The method of claim 8, wherein the pharmaceutical composition has a pH in the range from pH 3.5 to pH 7.

16. The method of claim 8, wherein the osmolarity of the pharmaceutical composition is in the range from 150 to 550 mOsM.

17. The method of claim 8, wherein the pharmaceutical composition is in the form of liquid droplets or solid particles.

18. The method of claim 17, wherein the majority and/or mean size of the liquid droplets or solid particles range in size from 20 microns to 50 microns.

19. The method of claim 8, wherein the pharmaceutical composition comprises at least one absorption enhancer.

20. A method of reducing glucose production in a mammal, the method comprising intranasally administering a K_{ATP} channel activator to the hypothalamus of the mammal in an amount effective to reduce glucose production in the mammal.

21-38. (canceled)

39. A method of reducing gluconeogenesis in the liver of a mammal, the method comprising intranasally administering a K_{ATP} channel activator to the hypothalamus of the mammal in an amount effective to reduce hepatic gluconeogenesis in the mammal.

40. A method of reducing serum triglyceride levels in a mammal, the method comprising intranasally administering a K_{ATP} channel activator to the hypothalamus of the mammal in an amount effective to reduce serum triglyceride levels in the mammal.

41-53. (canceled)

54. A method of reducing serum very low density lipoprotein (VLDL) levels in a mammal, the method comprising intranasally administering a K_{ATP} channel activator to the hypothalamus of the mammal in an amount effective to reduce serum VLDL levels in the mammal.

55-67. (canceled)

68. A method of treating a disorder in a mammal selected from the group consisting of obesity, type 2 diabetes, type 1 diabetes, hyperglycemia, insulin resistance, glucose intolerance, leptin resistance, metabolic syndrome, heart failure, ischemia, coronary heart disease, familial lipoprotein lipase deficiency, hypopituitarism, hyperlipidemia, hypertriglyceridemia, hyperVLDLemia, atherosclerosis, hypercholesterolemia, hypertension, and any combination of the foregoing, comprising intranasally administering a K_{ATP} channel activator to the hypothalamus of the mammal in an amount effective to treat the disorder.

69-85. (canceled)

86. A method of increasing K_{ATP} channel activity in the hypothalamus of a mammal, the method comprising intranasally administering a K_{ATP} channel activator to the mammal in an amount effective to increase K_{ATP} channel activity in the hypothalamus.

87-96. (canceled)

97. A pharmaceutical composition formulated for intranasal administration comprising a K_{ATP} channel activator in a pharmaceutically acceptable carrier.

98. A method of increasing peripheral blood glucose levels in a mammal, the method comprising intranasally administering a K_{ATP} channel inhibitor to the hypothalamus of the mammal in an amount effective to increase peripheral blood glucose levels in the mammal.

99-103. (canceled)

104. A pharmaceutical composition formulated for intranasal administration comprising a K_{ATP} channel activator in a pharmaceutically acceptable carrier.
